SEQUENCE LISTING (54 sequences)

SEQ ID NO: 1 - protein, 9 aa, Artificial Sequence
  Chemically synthesized peptide
APAPAPEER

SEQ ID NO: 2 - protein, 9 aa, Artificial Sequence
  Chemically synthesized peptide
APAPAPEER

SEQ ID NO: 3 - protein, 8 aa, Artificial Sequence
  Chemically synthesized peptide
ILNPGEYR

SEQ ID NO: 4 - protein, 8 aa, Artificial Sequence
  Chemically synthesized peptide
ILNPGEYR

SEQ ID NO: 5 - protein, 14 aa, Artificial Sequence
  Chemically synthesized peptide
NPFVLGANLNGGER

SEQ ID NO: 6 - protein, 14 aa, Artificial Sequence
  Chemically synthesized peptide
NPFVLGANLNGGER

SEQ ID NO: 7 - protein, 15 aa, Artificial Sequence
  Chemically synthesized peptide
DTPVLSELPEPVVAR

SEQ ID NO: 8 - protein, 15 aa, Artificial Sequence
  Chemically sythesized peptide
DTPVLSELPEPVVAR

SEQ ID NO: 9 - protein, 11 aa, Artificial Sequence
  Chemically synthesized peptide
VVNEEXPTITR

SEQ ID NO: 10 - protein, 11 aa, Artificial Sequence
  Chemically synthesized peptide
VVNEEXPTITR

SEQ ID NO: 11 - protein, 15 aa, Artificial Sequence
  Chemically synthesized peptide
AAPAPAPPPEPERPK

SEQ ID NO: 12 - protein, 15 aa, Artificial Sequence
  Chemically synthesized peptide
AAPAPAPPPEPERPK

SEQ ID NO: 13 - protein, 13 aa, Artificial Sequence
  Chemically synthesized peptide
ALGQNPTQAEVLR

SEQ ID NO: 14 - protein, 13 aa, Artificial Sequence
  Chemically synthesized peptide
ALGQNPTQAEVLR

SEQ ID NO: 15 - protein, 8 aa, Artificial Sequence
  Chemically synthesized peptide
EVEFDASK

SEQ ID NO: 16 - protein, 8 aa, Artificial Sequence
  Chemically synthesized peptide
EVEFDASK

SEQ ID NO: 17 - protein, 9 aa, Artificial Sequence
  Chemically synthesized peptide
HVLATLGER

SEQ ID NO: 18 - protein, 9 aa, Artificial Sequence
  Chemically synthesized peptide
HVLATLGER

SEQ ID NO: 19 - protein, 12 aa, Artificial Sequence
  Chemically synthesized peptide
AIVAGDQNVEYK

SEQ ID NO: 20 - protein, 12 aa, Artificial Sequence
  Chemically synthesized peptide
AIVAGDQNVEYK

SEQ ID NO: 21 - protein, 8 aa, Artificial Sequence
  Chemically synthesized peptide
NPITGFGK

SEQ ID NO: 22 - protein, 8 aa, Artificial Sequence
  Chemically synthesized peptide
NPITGFGK

SEQ ID NO: 23 - protein, 10 aa, Artificial Sequence
  Chemically synthesized peptide
FXANTXVEXR

SEQ ID NO: 24 - protein, 10 aa, Artificial Sequence
  Chemically synthesized peptide
FXANTXVEXR

SEQ ID NO: 25 - protein, 9 aa, Artificial Sequence
  Chemically synthesized peptide
DXFTXSNXK

SEQ ID NO: 26 - protein, 9 aa, Artificial Sequence
  Chemically synthesized peptide
DXFTXSNXK

SEQ ID NO: 27 - protein, 11 aa, Artificial Sequence
  Chemically synthesized peptide
QLEDELVSLQK

SEQ ID NO: 28 - protein, 11 aa, Artificial Sequence
  Chemically synthesized peptide
QLEDELVSLQK

SEQ ID NO: 29 - protein, 10 aa, Artificial Sequence
  Chemically synthesized peptide
LVIIESDLER

SEQ ID NO: 30 - protein, 10 aa, Artificial Sequence
  Chemically synthesized peptide
LVIIESDLER

SEQ ID NO: 31 - protein, 10 aa, Artificial Sequence
  Chemically synthesized peptide
ESDTSYVSLK

SEQ ID NO: 32 - protein, 10 aa, Artificial Sequence
  Chemically synthesized peptide
ESDTSYVSLK

SEQ ID NO: 33 - protein, 10 aa, Artificial Sequence
  Chemically synthesized peptide
GYSIFSYATK

SEQ ID NO: 34 - protein, 10 aa, Artificial Sequence
  Chemically synthesized peptide
GYSIFSYATK

SEQ ID NO: 35 - protein, 13 aa, Artificial Sequence
  Chemically synthesized peptide
LGHPDTLNQGEFK

SEQ ID NO: 36 - protein, 13 aa, Artificial Sequence
  Chemically synthesized peptide
LGHPDTLNQGEFK

SEQ ID NO: 37 - protein, 10 aa, Artificial Sequence
  Chemically synthesized peptide
IANVFTNAFR

SEQ ID NO: 38 - protein, 10 aa, Artificial Sequence
  Chemically synthesized peptide
IANVFTNAFR

SEQ ID NO: 39 - protein, 8 aa, Artificial Sequence
  Chemically synthesized peptide
TLLLQIAK

SEQ ID NO: 40 - protein, 8 aa, Artificial Sequence
  Chemically synthesized peptide
TLLLQIAK

SEQ ID NO: 41 - protein, 11 aa, Artificial Sequence
  Chemically synthesized peptide
NITEIADLTQK

SEQ ID NO: 42 - protein, 11 aa, Artificial Sequence
  Chemically synthesized peptide
NITEIADLTQK

SEQ ID NO: 43 - protein, 13 aa, Artificial Sequence
  Chemically synthesized peptide
VLAIDHLNEDQLR

SEQ ID NO: 44 - protein, 13 aa, Artificial Sequence
  Chemically synthesized peptide
VLAIDHLNEDQLR

SEQ ID NO: 45 - protein, 7 aa, Artificial Sequence
  Chemically synthesized peptide
YEINVLR

SEQ ID NO: 46 - protein, 7 aa, Artificial Sequence
  Chemically synthesized peptide
YEINVLR

SEQ ID NO: 47 - protein, 8 aa, Artificial Sequence
  Chemically synthesized peptide
EVATEGIR

SEQ ID NO: 48 - protein, 8 aa, Artificial Sequence
  Chemically synthesized peptide
EVATEGIR

SEQ ID NO: 49 - protein, 7 aa, Artificial Sequence
  Chemically synthesized peptide
MVLYTLR

SEQ ID NO: 50 - protein, 7 aa, Artificial Sequence
  Chemically synthesized peptide
MVLYTLR

SEQ ID NO: 51 - protein, 7 aa, Artificial Sequence
  Chemically synthesized peptide
MVLYTLR

SEQ ID NO: 52 - protein, 7 aa, Artificial Sequence
  Chemically synthesized peptide
MVLYTLR

SEQ ID NO: 53 - protein, 11 aa, Artificial Sequence
  Chemically synthesized peptide
VVNEECPTITR

SEQ ID NO: 54 - protein, 10 aa, Artificial Sequence
  Chemically synthesized peptide
FCANTCVECR

(19) United States
(12) Patent Application Publication
(10) Pub. No.: US 2014/0045714 A1
(43) Pub. Date: Feb. 13, 2014

(54) Novel Biomarkers For Cardiovascular Injury

(71) Applicants: Robert Gerszten, Brookline, MA (US); Michael Fifer, Brookline, MA (US); Steven A. Carr, Boxford, MA (US)

(72) Inventors: Robert Gerszten, Brookline, MA (US); Michael Fifer, Brookline, MA (US); Steven A. Carr, Boxford, MA (US)

(21) Appl. No.: 13/881,327

(22) PCT Filed: Oct. 26, 2011
(86) PCT No.: PCT/US11/57894

§ 371 Date: Oct. 22, 2013

Related U.S. Application Data
(60) Provisional application No. 61/407,345, filed on Oct. 27, 2010.

(51) Int. Cl. G01N 33/68

(52) U.S. Cl. CPC G01N 33/6887
USPC 506/9; 506/12; 435/7.92; 435/7.4; 435/6.11; 435/6.12; 435/287.2; 506/18

(57) ABSTRACT

The invention provides methods for the early detection of cardiovascular injury using one or more cardiac injury biomarkers identified herein.

### RELATED APPLICATIONS

This application claims priority to U.S. Ser. No. 61/407,345, filed on Oct. 27, 2010, which is herein incorporated by reference in its entirety.

### GOVERNMENT INTEREST STATEMENT

This invention was made with government support under R01 HL096738-01 awarded by the National Institutes of Health. The government has certain rights in the invention.

### FIELD OF THE INVENTION

The present invention relates to the identification of novel early biomarkers for diagnosis and identification of cardiovascular injury and to the use of a proteomics-based verification pipeline to identify early biomarkers of cardiovascular injury.

### BACKGROUND OF THE INVENTION

Despite frequent reports of the discovery of new potential protein biomarkers from proteomic studies, including many studies in cardiovascular biology (see Edwards et al., Mol. Cell Proteomics 7:1824-37 (2008); Jacquet et al., Mol. Cell. Proteomics 7:1824-37 (2009); and Fu et al., Expert Rev Proteomics 237-249 (2006)), none have been introduced into clinical use. In fact, the overall rate of introduction of new protein biomarkers into clinical use has been static at approximately one to two per year for the past 15 years. (See Anderson et al., Clin Chem 56:177-85 (2010); Kulasingam et al., Nature Clin Practice Oncol 5:588-99 (2008); and Rifai et al., Nat. Biotechnol 24:971-983 (2006)). The reasons for this lack of facile translation from discovery into clinical implementation is that discovery “omics” experiments do not lead to biomarkers of immediate clinical utility, but rather produce “candidates” that must be further credentialed with respect to their ability to distinguish presence or stage of disease from healthy or “at risk” controls. Many differentially-abundant proteins observed in clinical proteomics discovery experiments are likely to be false discoveries given the large number of hypotheses being tested simultaneously and the small numbers of samples used in the resource-intensive discovery phase, compounded by technical irreproducibility and biological inter-individual variability. (See Rifai et al., Nat. Biotechnol. 24:971-83 (2006); Paulovich et al., Proteomics Clin. Appl. 2:1386-1402 (2008)). To date, no coherent strategy has emerged for progressively credentialing putative protein biomarkers from discovery to initial clinical validation. Thus, there exists a need for the development of methods to measure large numbers of candidate proteins observed to be differentially abundant.

Early detection of cardiovascular injury allows for a more effective therapeutic treatment with a correspondingly more favorable clinical outcome. In many cases, however, early detection of cardiovascular disease is problematic. Clinical investigation of cardiovascular biomarkers over the past decade has led to the establishment of the cardiac troponins as the cornerstone for the diagnosis of acute myocardial infarction (AMI). (See Jaffe et al., Circulation 102:1216-20 (2000)) However, significant elevation of troponin level is not apparent until four to six hours after the onset of an acute coronary syndrome (ACS). (See Zimmerman et al., Circulation 99:1671-77 (1999))

Furthermore, although several markers of irreversible myocardial necrosis have been identified, a major current deficiency is that there are currently no satisfactory markers of reversible myocardial ischemia. (See Morrow et al., Clin Chem 49:537-39 (2003)) Development of such markers would permit biochemical confirmation of unstable angina, which must currently be diagnosed by a combination of a history consistent with typical angina pectoris, and labile electrocardiographic (ECG) ST-segment and T wave changes. (See Braunwald et al., Circulation 90:613-22 (1994)) This approach, however, is often unsatisfactory because of the transient nature of electrocardiographic changes and the subjective nature of history-taking, particularly in the ever-increasing subsets of elderly and diabetic patients. Faced with these limitations, physicians will typically order a stress test to help confirm or exclude the diagnosis of myocardial ischemia. However, this approach also has its limitations. A standard exercise stress test has a sensitivity of only 60% (and less than 50% for single-vessel disease) and a specificity of only 70%. (See Gibbons et al., Journal of the American College of Cardiology 30:260-311 (1997); Gianrossi et al., Circulation 80:87-98 (1989)) The addition of myocardial perfusion imaging with agents such as ²⁰¹ thallium or ^{99m}Tc-sestaMIBI improves the operating characteristics of the test, but adds over $2500 to the cost. (See Ritchie et al., Journal of the American College of Cardiology 25:521-47 (1995)) In addition to myocardial ischemia, other pathophysiological pathways are in need of reliable biochemical detection, including endothelial cell dysfunction, oxidative stress, and platelet aggregation.

Mounting evidence supporting early intervention for patients across the spectrum of ACS (see Boden et al., New Eng. J. Med. 360:2165-75 (2009); Cannon et al., New Eng. J. Med 344:1879-87 (2001); Neumann et al., J. Amer. Med. Assoc. 290:1593-99 (2003)) suggests that novel biomarkers that provide biochemical proof of early myocardial injury could have a substantial positive impact on patient care. Furthermore, it has been hypothesized that simultaneous assessment of biomarkers representing distinct biological axes triggered by AMI, such as myocyte necrosis, ventricular wall stress, or inflammation, will offer complementary prognostic information. This might enable clinicians to risk stratify patients with acute coronary syndromes more effectively (see Sabitine et al., Circulation 105:1760-63 (2002)), and could suggest targets for potential therapeutic manipulation.

Thus, there exists a need for sensitive and specific clinical assessments of early cardiovascular injury. The identification of novel early cardiovascular biomarkers that are specific for cardiovascular injury would prove immensely beneficial for both prediction of outcome and for targeted therapy.

### SUMMARY OF THE INVENTION

The invention provides methods for detecting or diagnosing cardiovascular injury in a subject by obtaining a biological sample from the subject; determining the level of expression of at least one biomarker selected from the group consisting of proteins 8-31 from Table 1B, the proteins of Table 1A, and any combination thereof, and comparing expression levels of the at least one biomarker or combination thereof in a reference or control sample. Those skilled in the art will recognize that a change in the expression level of at least one biomarker or combination thereof as compared to the reference or control is indicative of cardiovascular injury in the subject. These methods can also include the step of additionally determining the level of expression of at least one additional biomarker selected from the group consisting of proteins 1-7 of Table 1B, or any combination thereof. For example, the levels of expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and/or more of the biomarkers can be determined.

Also provided herein are methods for obtaining an indication useful in detecting or diagnosing cardiovascular injury in a subject comprising the steps of: a) determining the level of expression of at least one biomarker selected from the group consisting of proteins 8-31 from Table 1B and the proteins of Table 1A and any combinations thereof, in a biological sample obtained from the subject; and b) comparing the expression levels of the at least one biomarker or combination thereof in a) with the expression levels of the same at least one biomarker or combination thereof in a reference or control sample; whereby a change in the expression level of the at least one biomarker or combination thereof, as compared to the reference or control sample, is indicative of cardiovascular injury in the subject.

Moreover, the invention also provides methods for obtaining indications useful in detecting or diagnosing cardiovascular injury in a subject comprising the steps of: a) determining the level of expression of at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (i.e., all)) of the biomarkers in the group consisting of proteins 8-31 from Table 1B and the proteins of Table 1A, in a biological sample obtained from the subject; and b) comparing expression levels of the biomarkers in a) with expression levels of the same biomarkers in a reference or control sample; whereby changes in the expression levels of the biomarkers, as compared to the reference or control sample, is indicative of cardiovascular injury in the subject.

In any of the methods described herein, determining the level of expression of at least one biomarker includes detecting the presence or absence of the at least one biomarker combination thereof and/or quantifying the level of expression of the at least one biomarker or combination thereof.

Levels of expression (and/or changes in the level of expression) can be detected by any method known to those in the art, including, but not limited to, polymerase chain reaction (PCR), microarray assay, or immunoassay. For example, the levels of expression can be detected by quantitative real-time RT-PCR.

In any of the methods described herein, determining the level of expression of the at least one biomarker or combination thereof occurs by detecting the expression, if any, of mRNA expressed by said biomarker or combination thereof in the sample. For example, determining the expression of mRNA can be achieved by exposing the sample to a nucleic acid probe complementary to said mRNA and quantifying the level of mRNA in the sample. Likewise, determining the level of expression of the at least one biomarker can involve detecting the expression, if any, of the polypeptide(s) encoded by said biomarker or combination thereof in the sample. For example, detecting the expression of the polypeptide(s) can be achieved by exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.

Those skilled in the art will appreciate that any of the methods of the present invention are preferably in vitro or ex vivo methods.

Also provided herein are methods for detecting or diagnosing cardiovascular injury in a subject by obtaining a biological sample from the subject; determining the level of expression of two or more cardiovascular injury biomarkers; and comparing expression levels of the two or more cardiovascular injury biomarkers in a reference or control sample, whereby a change in the expression level of the two or more cardiovascular injury biomarkers as compared to the reference or control is indicative of cardiovascular injury in the subject.

The invention further provides methods for obtaining indications useful in detecting or diagnosing cardiovascular injury in a subject comprising the steps of: a) determining the level of expression of two or more cardiovascular injury biomarkers in a biological sample obtained from the subject; and b) comparing expression levels of the two or more cardiovascular injury biomarkers in a) with the expression levels of the same two or more cardiovascular injury biomarkers in a reference or control sample; whereby a change in the expression level of the two or more cardiovascular injury biomarkers as compared to the reference or control sample is indicative of cardiovascular injury in the subject.

For example, in these methods, the two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and/or more) cardiovascular injury biomarkers are selected from the proteins listed in Table 1A, Table 1B, and/or Table 4 (or any combination(s) thereof).

Those skilled in the art will recognize that determining the level of expression of a biomarker may include detecting the presence or absence of the two or more cardiovascular injury biomarkers described herein and/or quantifying the level of expression of the two or more cardiovascular injury biomarkers described herein.

Levels of expression can be detected by any method known to those in the art, including, but not limited to, polymerase chain reaction (PCR), microarray assay, or immunoassay. For example, the levels of expression can be detected by quantitative real-time RT-PCR.

Determining the level of expression of the two or more cardiovascular injury biomarkers occurs by detecting the expression, if any, of mRNA expressed by the biomarkers in the sample. For example, determining the expression of mRNA can be achieved by exposing the sample to a nucleic acid probe complementary to said mRNA and quantifying the level of mRNA in the sample.

Likewise, determining the level of expression of the two or more cardiovascular injury biomarkers can involve detecting the expression, if any, of the polypeptide(s) encoded by the biomarkers in the sample. For example, detecting the expression of the polypeptide(s) can be achieved by exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.

By way of non-limiting example, in any of the methods described herein, the biological sample comprises whole blood, blood fraction, plasma, or a fraction thereof.

Moreover, in any of the methods disclosed herein, the cardiovascular injury can include, but is not limited to, myocardial infarction, stable ischemic heart disease, unstable ischemic heart disease, acute coronary syndrome, ischemic cardiomyopathy, and heart failure.

Also provided herein are kits containing, in one or more containers, at least one of the proteins listed in Table 1A, Table 1B, or Table 4, wherein the level of expression of the proteins can be determined using the components of the kit. Such kits can be used to generate a biomarker profile, and may, optionally, also contain at least one internal standard to be used to generate the biomarker profile. Moreover, in some embodiments, the kit can also contain at least one pharmaceutical excipient, diluent, adjuvant, or any combination thereof.

The invention further provides kits containing, in one or more containers, at least one detectably labeled reagent that specifically recognize at least one of the proteins listed in Table 1A, Table 1B, and/or Table 4. By way of non-limiting example, the reagent may be one or more antibodies or antigen binding or functional fragments thereof; an aptamer; and/or an oligonucleotide probe that specifically bind to at least one of the proteins. In such kits, the at least one detectably labeled reagent is used to determine the expression level of at least one of the proteins listed in Table 1A, Table 1B, or Table 4 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, and/or more) in a biological sample, including, for example, whole blood, blood fraction, plasma, or a fraction thereof. The kits may also include written instructions for use thereof.

Also provided are methods of selecting an appropriate therapy or treatment protocol in a patient diagnosed with or suspected of having a cardiovascular injury by obtaining a biological sample from the subject; determining the level of expression of at least one (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and/or more) biomarker selected from the group consisting of proteins 8-31 from Table 1B, the proteins of Table 1A, and any combinations thereof; and choosing the appropriate therapy or treatment protocol based on the level of expression of the at least one biomarker or combination thereof.

Similarly, the invention also provides methods of obtaining indications useful in selecting an appropriate therapy or treatment protocol for a patient diagnosed with or suspected of having a cardiovascular injury, the method comprising: determining the level of expression of at least one biomarker (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and/or more) selected from the group consisting of proteins 8-31 from Table 1B and the proteins of Table 1A and any combinations thereof, in a biological sample obtained from the subject; wherein the level of expression of the at least one biomarker or combination thereof is indicative of the appropriate therapy or treatment protocol.

These methods can also be repeated on a periodic basis (e.g., hourly, daily, weekly, or monthly, etc.) in order to determine whether an additional and/or alternative therapy or treatment protocol needs to be chosen.

The invention also provides methods of identifying biomarker(s) (e.g., biomarker(s) of cardiovascular injury), by discovering one or more candidate biomarker proteins in proximal fluid or tissue; qualifying the one or more discovered candidate biomarker proteins in peripheral blood of additional patient samples; and verifying the qualified, discovered one or more candidate biomarker proteins. For example, the discovering of the one or more candidate biomarker proteins is accomplished using liquid chromatography-tandem mass spectrometry (LC-MS/MS) with extensive fractionation; the qualifying of the one or more discovered candidate biomarker proteins is accomplished using Accurate Inclusion of Mass Screening (AIMS); and the verifying of the qualified, discovered one or more candidate biomarker proteins is accomplished using targeted, qualitative a MS-based assay, such as multiple reaction monitoring mass spectrometry (MRM-MS) and/or SISCAPA.

Finally, the invention also provides methods for detecting or diagnosing cardiovascular injury in a subject by obtaining a biological sample from the subject; determining the level of expression of Acyl-CoA binding protein (ACBP); and comparing expression levels of the Acyl-CoA binding protein (ACBP) to a reference or control sample, whereby a change in the expression level of Acyl-CoA binding protein (ACBP) as compared to the reference or control is indicative of cardiovascular injury in the subject. Such methods may additionally involve the step of determining the level of expression of at least one additional biomarker selected from the group consisting of proteins from Table 1A, the proteins of Table 1B, and any combination thereof.

Those skilled in the art will recognize that determining the level of expression of Acyl-CoA binding protein (ACBP) comprises detecting the expression, if any, of the polypeptide(s) encoded by Acyl-CoA binding protein (ACBP) in the sample. By way of non-limiting example, detecting the expression of the polypeptide(s) comprises exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.

In these methods, the biological sample can be whole blood, blood fraction, plasma, or a fraction thereof. Moreover, the cardiovascular injury may be myocardial infarction, stable ischemic heart disease, unstable ischemic heart disease, acute coronary syndrome, ischemic cardiomyopathy, heart failure, and myocardial ischemia. In one preferred embodiment, the cardiovascular injury is myocardial ischemia (i.e., exercise-induced myocardial ischemia).

The present invention is based upon the discovery of novel, sensitive biomarkers that provide biochemical evidence of early cardiovascular injury (e.g., myocardial injury). For example, any of the proteins identified in Tables 1A and/or 1B (alone or in any combination) may also be useful markers of cardiovascular injury or disease.

According to one embodiment, the methods of the present invention involve obtaining a profile of biomarkers from a biological sample obtained from an individual who is suspected of having experienced a cardiovascular injury or event. The biological sample may be whole blood, blood fraction, serum, plasma, blood cells, a muscle or tissue biopsy, and/or a cellular extract. Moreover, those skilled in the art will recognize that the biological sample may also be a proximal fluid, either natural (e.g., nipple aspirate fluid or cerebrospinal fluid (CSF)) or a pseudo-proximal fluid (e.g., tissue interstitial fluid that is prepared from fresh tissue that is incubated in buffer and then the soluble fraction containing the actively shed and secreted proteins constitutes the pseudo-proximal fluid). In a particular embodiment, the biological sample is a blood sample obtained from a site which is proximal to the cardiovascular injury. The reference biomarker profile may be obtained, for example, from the same subject prior to experiencing a cardiovascular injury or event, or from a normal, healthy subject.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from the following detailed description and claims.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is an overview of the discovery-through verification pipeline described herein and its application to a human model of myocardial injury to identify early biomarkers of cardiovascular injury. Blood samples were collected from the coronary sinus of patients undergoing alcohol septal ablation for hypertrophic cardiomyopathy (a.k.a. “planned” myocardial infarction or PMI) at baseline prior to ablation, and at 10 and 60 minutes post ablation. These samples represent proximal fluid and were used for discovery proteomics studies in which extensive fractionation and LC-MS/MS was performed to generate a prioritized list of biomarker candidates. Peripheral blood was collected from patients undergoing the procedure at the same time points an extending to 24 hours post ablation. Blood collected up to 4 hours post ablation were used for analytical qualification by Accurate Inclusion Mass Screening (AIMS), a process that determines which of the differentially abundant proteins from the discovery experiments are detectable in peripheral blood. Qualified protein biomarker candidates were subsequently quantitatively measured in peripheral blood using immunoassays when antibodies were available and multiple reaction monitoring mass spectrometry (MRM-MS) when antibody reagents were not available.

FIG. 2 is an overview of the sample preparation workflow for discovery proteomics (A), qualification by AIMS (B), verification by targeted, quantitative assays by MRM/MS (C), and verification by Western blot analysis and ELISA assays (D).

FIG. 3 summarizes the assay configuration and sample preparation workflow for multiple reaction monitoring mass spectrometry with stable isotope dilution. Workflow (A) represents the method used to select signature peptides for proteins associated with cardiac injury. Workflow (B) represents assay configuration conducted in parallel for MS instrument optimization and peptide separation by SCX chromatography. Workflow (C) represents the plasma processing and limited fractionation/MRM assay employed for all 4 patients and time points (baseline and 10, 60, and 240 minutes post ablation). Three process replicates for all samples were performed.

FIG. 4 shows Venn diagrams summarizing proteins identified in the coronary sinus of PMI patients. (a), (b), and (c) show the overlap of proteins identified across all 3 time points in patients 1, 2 and 3, respectively. Proteins were identified with a minimum of 2 unique peptides per protein and a peptide false discovery rate (FDR) of ≦1%. A total of 1086 unique proteins were identified in the nine coronary sinus samples analyzed by LC-MS/MS with >70% of the proteins identified in common across the 3 patients (d). Label free, relative quantitation of peptides was performed in order to prioritize candidate proteins for subsequent qualification and verification studies. A minimum of a five-fold change in the MS-derived discovery data between baseline and either the 10 minute or 60 minute time point was required. 121 proteins met these criteria in all 3 or any 2 patients combined (e).

FIG. 5 is a bar graph showing a summary of the total number of unique proteins identified across all time points in 3 planned myocardial infarctions (PMI) from proteomics studies. Proteins were identified with a minimum of 2 distinct peptides per protein and with a peptide false discovery rate of <2%.

FIG. 6 depicts bar graphs of the kinetic analyses of known (a) and putative (b) biomarkers for acute myocardial infarction in 3 PMI patients from discovery proteomics. (a) Known markers, such as creatine kinase M-type, myoglobin, myeloperoxidase, and fatty acid binding protein 3, showed little to no detection at baseline in CS followed by an increase of greater than 5-fold at 10 minutes and 60 minutes post ablation in 3 PMI patients. Panel (b) shows 8 new candidate biomarkers from discovery proteomics. These proteins showed no to little detection at baseline in CS then increased by a minimum of 5-fold in MS abundance at 10 minutes or 60 minutes post ablation in all 3 PMI patients. MRM-MS assays were configured for aortic carboxypeptidase-like protein 1, myosin light chain 3, and four-and-a-half LIM domain protein 1 to quantify these candidates in peripheral plasma of 4 PMI patients. Antibodies available for acyl-CoA-binding protein, angiogenin, midkine, malate dehydrogenase, and aortic carboxypeptidase-like protein 1 were used either in ELISA assays or Western blot analyses to verify these candidates in additional patients.

FIG. 7 depicts bar graphs of normalized MS intensities for 42 proteins detected in three discrete pools of peripheral plasma from 10 PMI patients from AIMS. An inclusion list of 1152 entries (m/z, z pairs) representing 82 proteins that increased ≧5-fold in MS abundance in the discovery data was generated for qualification by AIMS in the baseline, 10 minute and 60 minute pools of peripheral plasma. Unique peptides derived from 42/82 proteins (51%) were detected and sequenced by AIMS in a pool of peripheral plasma from 10 PMI patients. For a majority of detected proteins, the relative quantitative information and temporal trends were consistent with that obtained by discovery proteomics of plasma from the coronary sinus of individual PMI patients.

FIG. 8 depicts line graphs for the verification of novel candidate biomarkers in peripheral blood of PMI patients by targeted, quantitative MS. Multiplexed SID-MRM-MS-based assays were configured for four candidate proteins in order to precisely quantify their changes in peripheral blood from PMI patients at 10 min, 60 min and 240 min post ablation. Multiple signature peptides derived from each protein were used to quantify protein levels (Table 2). Measured concentrations for the four novel proteins ranged from 1 ng/mL to ˜50 ng/mL across all patients and time points. Error bars indicate standard error of the mean concentration measured at each time point. Signature peptides are represented by the first four residues. ACLP1=aortic carboxypeptidase-like protein 1; FHL1=four-and-a-half LIM domain protein 1; MYL3=myosin light chain 3; TPM1=tropomyosin 1.

FIG. 9 depicts the verification of candidate biomarkers by Western blot analysis and ELISA assay. (Panel a) Single antibody reagents suitable for Western blot analysis were available for midkine (MDK), pleiotrophin (PTN), malate dehydrogenase 1 (MDH1) and aortic carboxypeptidase-like protein 1 (ACLP1). Kinetic analysis of CS samples from 6 patients show consistency in the protein changes between the Western blot results shown here and the MS-derived temporal trends shown in FIG. 6 for the identical proteins. (Panel b) For angiogenin (ANG), acyl CoA binding protein (ACBP), and C-C motif chemokine 21 (CCL21), sandwiched immunoassays were either constructed (ANG) or commercially available (ACBP and CCL21), and were used to verify protein changes in peripheral plasma from a larger set of PMI patient samples, control samples and spontaneous MI cohorts. In the PMI cohort. (Panel b, left) ELISA results confirm significant changes in these candidate biomarkers as early as 10 minutes after the onset of myocardial injury. In patients with spontaneous MI (panel b, right) presenting for acute coronary angiography and intervention, significantly higher levels of these proteins were observed as compared to levels in patients who presented to the cardiac catheterization suite with non-acute coronary artery disease (controls, panel b center). NS=not significant.

FIG. 10 depicts line graphs for the verification of candidate biomarkers in patients undergoing exercise stress testing. A total of 52 patients undergoing exercise stress testing with myocardial perfusion imaging served as the study population: 26 with no evidence of ischemia (controls) and 26 patients with evidence of inducible ischemia (cases). For ACBP and ANG, baseline levels were higher in the ischemic as compared to the at-risk control patients. Furthermore, for ACBP, a modest augmentation in protein levels was documented in the setting of myocardial ischemia that was not observed in the control subjects.

FIG. 11 is a graph showing the results of ROC curve analyses, which confirmed that Acyl-CoA binding protein (ACBP) levels were a strong predictor of ischemic class (ischemia vs. no ischemia).

### DETAILED DESCRIPTION

The present invention identifies novel, sensitive and specific biomarkers that are diagnostic of early cardiovascular injury. Detection of different early cardiovascular biomarkers according to the invention is also diagnostic of the degree of severity of injury, the cell(s) involved in the injury, and/or the localization of the injury. Advantageously, using the methods disclosed herein, cardiovascular injury may be detected within minutes following an acute cardiovascular event, thereby allowing for more effective therapeutic intervention.

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims.

In the specification and the appended claims, the singular forms include plural references unless the context clearly dictates otherwise. For convenience, certain terms used in the specification, examples and claims are collected here. Prior to setting forth the invention, it may be helpful to an understanding thereof to set forth definitions of certain terms that will be used hereinafter.

A “biomarker” in the context of the present invention is a molecular indicator of a specific biological property; for example, a biochemical feature or facet that can be used to detect cardiovascular injury. As used herein, the terms “biomarker” or “biomarkers” and the like encompass, without limitation, genes, proteins, nucleic acids (e.g., circulating nucleic acids (CNA)) and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Those skilled in the art will recognize that the biomarkers (e.g., genes, proteins, nucleic acids, and/or metabolites) can be used to detect, diagnose, and/or monitor the onset and/or severity of cardiovascular injury.

A combination of biomarkers, or “profile” can include a validated selection of optimal biomarkers. Selection of an effective set of optimal biomarkers involves differentiating which genes are particularly indicative of cardiovascular injury.

“Detect” or “detection” refers to identifying the presence, absence or amount of the object to be detected. A “biological sample” or “sample” in the context of the present invention is a biological sample isolated from a subject and can include, by way of non-limiting example, whole blood, blood fraction, serum, plasma, cerebrospinal fluid (CSF), urine, saliva, sputum, ductal fluid, bronchioaveolar lavage, blood cells, tissue biopsies, a cellular extract, a muscle or tissue sample, a muscle or tissue biopsy, or any other secretion, excretion, or other bodily fluids, including proximal fluids such as nipple aspirate fluid, synovial fluid, ductal lavage and pseudo-proximal fluids such as tissue interstitial fluid (see Celis et al., Mol. Cell. Proteomics 3:327-44 (2004) (incorporated herein by reference)). Samples can be taken from a subject at defined time intervals (e.g., hourly, daily, weekly, or monthly) or at any suitable time interval as would be performed by those skilled in the art.

A “control” or a “reference” subject in the context of the present invention encompasses the same subject assessed at least two different time points, or a normal or healthy subject (i.e., a subject that has not experienced or is not at risk for experiencing a cardiovascular injury).

A “control” or a “reference” sample as used in the context of the present invention encompasses: a) a biological sample obtained from the same individual, provided that the test and control or reference samples are taken at different time points; or b) a biological sample obtained from a normal, healthy subject ((i.e., one who has not experienced or is not at risk for experiencing a cardiovascular injury) appropriately matched with respect to age and sex to the case sample. The terms “control sample”, “reference sample” and the like are used interchangeably herein

A “decision rule” is a method used to classify patients. This rule can take on one or more forms that are known in the art, as exemplified in Hastie et al., in “The Elements of Statistical Learning,” Springer-Verlag (Springer, N.Y. (2001)), herein incorporated by reference in its entirety. Analysis of biomarkers in the complex mixture of molecules within the sample generates features in a data set. A decision rule may be used to act on a data set of features to, inter alia, detect or diagnose a cardiovascular injury or event.

As used herein, the phrases “change in the expression levels” or “changes in the expression levels” (or the like) refers to a difference (i.e., an increase and/or a decrease) in the expression levels of one or more of the biomarkers described herein. For example, the phrase “differentially expressed” refers to differences in the quantity and/or the frequency of a biomarker present in a sample taken from patients having, for example, myocardial injury, as compared to a control subject. For example, without limitation, a biomarker can be a polypeptide which is present at an elevated level or at a decreased level in samples of patients with myocardial injury as compared to samples of control subjects. Alternatively (or additionally), a biomarker can be a polypeptide which is detected at a higher frequency or at a lower frequency in samples of patients compared to samples of control subjects. A biomarker can be differentially present in terms of quantity, frequency or both.

A biomarker is differentially present between the two samples if the amount of the biomarker in one sample is statistically significantly different from the amount of the biomarker in the other sample. For example, a biomarker is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively (or additionally), a biomarker is differentially present between the two sets of samples if the frequency of detecting the biomarker in samples of patients suffering from for example, myocardial injury, is statistically significantly higher or lower than in the control samples. For example, a biomarker is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “algorithms” include sums, ratios, and regression operators, such as coefficients or exponents, biomarker value transformations and normalizations (including, without limitation, those normalization schemes based on clinical parameters, such as gender, age, smoking status, or ethnicity), rules and guidelines, statistical classification models, and neural networks trained on historical populations. Of particular use in combining the biomarkers of the present invention are linear and non-linear equations and statistical classification analyses to determine the relationship between levels of biomarkers detected in a subject sample.

For complex statistical data analysis derived from the disclosed composition and methods, Principal Component Analysis (PCA) can be generally applied, however any algorithm or computed index can be used, such as but not limited to, cross-correlation, factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, Leave-One-Out (LOO), 10-Fold cross-validation (10-Fold CV), and Hidden Markov Models, among others.

As used herein, the term “injury” or “cardiovascular injury” is intended to include any damage which directly or indirectly affects the normal functioning of the cardiovascular system. By way of non-limiting example, the injury can be damage to the heart due to myocardial infarction (including non-ST segment elevation myocardial infarction (NSTEMI) and ST segment elevation myocardial infarction (STEMI)), acute coronary syndrome, stable ischemic heart disease, unstable ischemic heart disease, ischemic cardiomyopathy, or heart failure.

“Measuring” or “measurement” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters. Measurement or measuring may also involve qualifying the type and/or identifying the biomarker(s). Measurement of the biomarkers of the invention may be used to diagnose, detect, or identify cardiovascular injury in a subject and/or to monitor the progression or prognosis of cardiovascular injury in a subject.

The terms “polypeptide,” “peptide” and “protein” are used interchangeably herein to refer to a polymer of amino acid residues. These terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

The term “proximal biological sample” as used herein is intended to refer to a biological sample which is nearer or nearest to the origin or site of cardiovascular injury.

The term “peripheral biological sample” as used herein is intended to refer to a biological sample located away from the origin or site of cardiovascular injury.

“Solid support” refers to a solid material which can be derivatized with, or otherwise attached to, a capture reagent. Exemplary solid supports include probes, microtiter plates, beads, and chromatographic resins. A similar term in the context of the present invention is “adsorbent surface”, which refers to a surface to which is bound an adsorbent (also called a “capture reagent” or an “affinity reagent”). An “adsorbent” is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. “Adsorption” refers to detectable non-covalent binding of an analyte to an adsorbent or capture reagent.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

A “subject” in the context of the present invention is preferably a mammal. The mammal can be a human, non-human primate, mouse, rat, dog, cat, horse, or cow, but are not limited to these examples. A subject can be male or female. A subject can be one who has been previously diagnosed or identified as having a cardiovascular injury, and optionally has already undergone, or is undergoing, a therapeutic intervention or treatment for the cardiovascular injury. Alternatively, a subject can also be one who has not been previously diagnosed as having a cardiovascular injury. For example, a subject can be one who exhibits one or more risk factors for cardiovascular injury, or a subject who does not exhibit risk factors for cardiovascular injury, or a subject who is asymptomatic for cardiovascular injury. A subject can also be one who is suffering from or at risk of developing cardiovascular injury, or who is suffering from or at risk of developing a recurrence of cardiovascular injury. A subject can also be one who has been previously treated for cardiovascular injury, whether by administration of therapeutic agents, surgery, or any combination of the foregoing.

The amount or expression level of the biomarker(s) can be measured in a test sample and compared to a “reference biomarker profile”, utilizing techniques such as reference limits, discrimination limits, or risk defining thresholds to define cutoff points and abnormal values for cardiovascular injury. The reference biomarker profile means the level of one or more biomarkers or combined biomarker indices typically found in a subject or reference population (which can include a single subject, at least two subjects, or any number of subjects including 20 subjects or more) not suffering from cardiovascular injury. Such reference biomarker profiles and cutoff points may vary based on whether a biomarker is used alone or in a formula combining with other biomarkers into a single value. Alternatively, the reference biomarker profile can be a database of biomarker patterns from previously tested subjects who did not experience cardiovascular injury over a clinically relevant time horizon.

Levels of an effective amount of one or more of the biomarkers described herein can then be determined and compared to a reference value, e.g. a control subject or population whose cardiovascular injury status is known, or an index value or baseline value. The reference sample or index value or baseline value may be taken or derived from one or more subjects who have been exposed to the treatment, or may be taken or derived from one or more subjects who are at low risk of developing cardiovascular injury, or may be taken or derived from subjects who have shown improvements in cardiovascular injury risk factors as a result of exposure to treatment. Alternatively, the reference sample or index value or baseline value may be taken or derived from one or more subjects who have not been exposed to the treatment. A reference value can also comprise a value derived from risk prediction algorithms or computed indices from population studies such as those disclosed herein.

The biomarkers of the present invention can thus be used to generate a reference biomarker profile of those subjects who do not have cardiovascular injury, and would not be expected to develop cardiovascular injury.

The biomarkers disclosed herein can also be used to generate a “subject biomarker profile” taken from subjects who have cardiovascular injury. The subject biomarker profiles can be compared to a reference biomarker profile to diagnose or identify subjects at risk for developing cardiovascular injury, to monitor the progression of disease, as well as the rate of progression of disease, and to monitor the effectiveness of cardiovascular injury treatment modalities or subject management.

The reference and subject biomarker profiles of the present invention can be contained in a machine-readable medium, such as but not limited to, analog or digital tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other risk algorithms and computed indices such as those described herein.

Differences in the genetic makeup of subjects can result in differences in their relative abilities to metabolize various drugs, which may modulate the symptoms or risk factors of cardiovascular injury. Subjects that have cardiovascular injury, or at risk for developing cardiovascular injury can vary in age, ethnicity, and other parameters. Accordingly, use of the biomarkers disclosed herein, both alone and together in combination with known clinical factors, allow for a pre-determined level of predictability that a putative therapeutic or prophylactic agent to be tested in a selected subject will be suitable for treating or preventing the cardiovascular injury in the subject.

To identify therapeutic agents or drugs that are appropriate for a specific subject, a test sample from the subject can also be exposed to a therapeutic agent or a drug, and the level of one or more biomarkers can be determined. The level of one or more biomarkers can be compared to sample derived from the subject before and after subject management for cardiovascular injury, e.g., treatment or exposure to a therapeutic agent or a drug, or can be compared to samples derived from one or more subjects who have shown improvements in cardiovascular injury risk factors as a result of such treatment or exposure.

The term “treating” in its various grammatical forms in relation to the present invention refers to preventing (e.g., chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

As used herein, the term “therapeutically effective amount” is intended to qualify a desired biological response, such as, e.g., is partial or total inhibition, delay or prevention of the progression of cardiovascular injury; inhibition, delay or prevention of the recurrence of cardiovascular injury; or the prevention of the onset or development of cardiovascular injury (e.g., chemoprevention) in a subject.

### Identification of Novel Early Biomarkers Indicative of Cardiovascular Injury

The present invention provides methods combining mass spectrometry and proteomics technologies to identify early biomarkers, which are indicative of a cardiovascular injury or event. The early sensitive and specific clinical assessment of cardiovascular injury has never previously been achieved in the art. The ability to detect and monitor levels of these proteins after cardiovascular injury provides enhanced diagnostic capability by allowing clinicians (1) to determine the level of injury severity in patients with various cardiovascular related injuries, (2) to monitor patients to signs of secondary cardiovascular injuries that may elicit these cellular changes, and (3) to monitor the effects of therapy by examination of these proteins in blood or plasma. Unlike other organ-based diseases where rapid diagnostics for surrogate biomarkers prove invaluable to the course of action taken to treat the disease, no such rapid, definitive diagnostic tests currently exist for acute ischemic cardiovascular injury that can provide physicians with quantifiable biochemical markers to help determine the seriousness of the injury, the anatomical and cellular pathology of the injury, and the implementation of appropriate medical management and treatment.

The methods of the present invention utilize a proteomics biomarker discovery-through-verification pipeline to identify early biomarkers of cardiovascular injury based on a biological sample obtained from a subject (e.g., blood, plasma or serum). Three distinct phases are employed in the discovery-through-validation pipeline described herein: a Discovery phase, a Qualification phase and a Verification phase.

In the Discovery phase, liquid chromatography-tandem mass spectrometry (LC-MS/MS)-based discovery protocols are used to identify low abundance constituents which are differentially expressed between a proximal biological sample obtained from individuals who experienced a cardiovascular injury or event and a control sample. LC/MS-MS is an unbiased discovery tool which uses new chromatographic techniques to deplete plasma samples of high abundance constituents and thus allows for differential analysis and identification of thousands of candidate proteins in human tissue or plasma. (See Brunner et al., Nat Biotechnol 25:576-83 (2007); Pagliarini et al., Cell 134:112-23 (2008)) In order to access proteins at lower abundance (e.g., sub 100 ng/mL in plasma, levels at which many known protein biomarkers such as carcinoembryonic antigen, PSA, and the troponins occur), the analyses employs multidimensional fractionation at the protein and/or peptide level, thus expanding a single patient sample into aliquots of up to a 100 sub-fractions for LC-MS/MS analysis.

A significant fraction of proteins “discovered” through the unbiased LS/MS-MS analysis are false positives arising from biological or technical variability. Thus, the candidate proteins that are identified must be qualified and verified. In the Qualification phase of the present invention, accurate inclusion mass screening (AIMS) is used to ascertain which of the candidate proteins identified in the proximal biological sample during the Discovery phase could also be detected in a peripheral biological sample. AIMS is a targeted MS approach in which an “inclusion list” is populated with the accurate masses of signature peptides derived from the high-priority candidate proteins from discovery experiments. (See Jaffe et al., Mol Cell Proteomics 7:1952-62 (2008)) Masses on the inclusion list are monitored in each scan on the MS system and MS/MS spectra are acquired only when a peptide from the list is detected with both the correct accurate mass and charge state. The use of AIMS to verify candidate proteins offers significant advantages over prior antibody-based methods used to validate candidate biomarker proteins. For example, the required immunoassay-grade Ab pairs exist for only a small number of the potential candidate biomarker proteins and the development of a new, clinically deployable immunoassay is expensive and time consuming, which restricts development to a short list of already highly credentialed candidates. In contrast, the use of AIMS enables rapid, sensitive, semi-quantitative qualification of ˜100 proteins/week in patient blood, involves low assay development cost, can be effectively multiplexed to analyze for 10-50 proteins in a single analysis, and involves low patient sample consumption (˜100-500 μL or less for the 10-50 proteins). More importantly, the use of AIMS enables one to triage (qualify or discard) a large number of biomarker candidates based on detection in plasma prior to committing to subsequent time and resource intensive steps.

A subset of the novel, candidate biomarkers, which are qualified using AIMS are next entered into a Verification phase. In the Verification phase, the qualified, novel candidate biomarkers are quantitatively assayed in blood using Stable Isotope Dilution (SID)-Multiple Reaction Monitoring (MRM)-Mass Spectrometry (MS) (see Anderson et al., Mol Cell Proteomics 5:573-88 (2006); Keshishian et al., Mol. Cell Proteomics 6:2212-29 (2007)) or ELISA in the minority of cases where Abs are available. The use of SID-MRM-MS for protein assays is predicated on measurement of “signature” or “proteotypic” tryptic peptides that uniquely and stoichiometrically represent the protein candidates of interest. In addition, proteins containing modifications such as phosphorylation or sequence isoforms or mutations can also be targeted by AIMS, thereby providing a rapid way to test for the presence of proteins containing these modifications in any matrix (tissue, cells or biofluids). MRM-based assay development starts with selection of 3-5 peptides per protein. (See Keshishian et al., Mol. Cell Proteomics 6:2212-29 (2007)) Synthetic, stable isotope-labeled versions of each peptide are used as internal standards, thereby enabling protein concentration to be measured by comparing the signals from the exogenous labeled and endogenous unlabeled species (differentiated in the mass spectrometer by the slight mass shift from the isotope). SID-MRM-MS assays have several distinguishing features relative to conventional immunoassays. First, the analyte detected in the MS can be characterized with near-absolute structural specificity, something never possible using antibodies alone, which provides a potentially critical quality advantage, especially in cases where immunoassays are subject to interferences. Second, MRM assays can be highly multiplexed such that dozens of proteins can be measured during a single analysis (See Anderson et al., Mol Cell Proteomics 5:573-88 (2006); Keshishian et al., Mol. Cell Proteomics 6:2212-29 (2007)), with excellent assay coefficients of variation (CVs; 100×Standard deviation/mean value of data set). (See Anderson et al., Mol Cell Proteomics 5:573-88 (2006)) Third, all of these measurements can be done on ˜100 μL of plasma.

Using the methods described above, the inventors of the present invention were the first to show that a combination of abundant protein depletion combined with minimal fractionation of tryptic peptides by strong cation exchange prior to SID-MRM-MS provides limits of quantitation (LOQs, signal to noise ratio of >10) in the 1-20 ng/mL range with CVs of 10-20% at the limits of quantitation for proteins in plasma (see Keshishian et al., Mol. Cell Proteomics 6:2212-29 (2007)). This breakthrough work has been extended to configure assays for early markers of cardiovascular disease (see Examples, infra) for which Ab reagents are not available.

The inventors applied a proteomics-based biomarker discovery-through-verification pipeline to identify early biomarkers of cardiovascular injury from blood samples of patients undergoing therapeutic, “planned” myocardial infarction (PMI) for hypertrophic cardiomyopathy. LC-MS/MS analyses detected 121 highly differentially expressed proteins across discovery patients, including previously credentialed markers of cardiovascular disease and many potentially novel biomarkers. After qualification with accurate inclusion mass screening, a subset of novel candidates were measured in peripheral plasma of patients with PMI or spontaneous MI and controls using quantitative, multiple reaction monitoring MS-based assays or immunoassays, and were shown to be specific to MI.

### Novel Early Biomarkers Indicative of Early Cardiovascular Injury

The biomarkers identified in accordance with the methods of the present invention allow one of skill in the art to identify, detect, diagnose, and/or otherwise assess those subjects who have experienced an acute cardiovascular injury or event within minutes after its occurrence. In one embodiment, the early biomarkers of the invention are capable of detecting a cardiovascular injury or event in a subject within minutes to hours after the onset of symptoms and/or after the occurrence of the cardiovascular injury or event. The biomarkers of the invention are also useful for guiding therapeutic intervention immediately following an acute cardiovascular injury or event (e.g., within minutes to hours post-injury or event).

Table 1A provides information (including a non-exhaustive list) regarding early biomarkers for detecting cardiovascular injury identified according to the methods described herein. Those skilled in the art will recognize that any of the biomarkers presented herein (alone or in any combination) can encompass all forms and variants thereof, including but not limited to, polymorphisms, isoforms, mutants, derivatives, precursors including nucleic acids and pro-proteins, cleavage products, receptors (including soluble and transmembrane receptors), ligands, protein-ligand complexes, and post-translationally modified variants (such as cross-linking or glycosylation), fragments, and degradation products, as well as any multi-unit nucleic acid, protein, and glycoprotein structures comprised of any of the biomarkers as constituent subunits of the fully assembled structure. All biomarker expression levels within blood samples have been validated through experimentation in accordance with the methods described herein.

TABLE 1A

| # | Candidate Biomarker Protein |
|---|---|
| 1 | ACLP—Aortic carboxypeptidase-like protein 1 |
| 2 | ANG—Angiogenin |
| 3 | CKB—Creatine kinase B-type |
| 4 | CKM—Creatine kinase M-type |
| 5 | FABP3—Fatty acid-binding protein, heart |
| 6 | FHL1—Four and a half LIM domains 1 |
| 7 | MB—Myoglobin |
| 8 | MPO—Isoform H7 of Myeloperoxidase |
| 9 | MYL3—Myosin light chain 3 |
| 10 | TPM1—Isoform 4 of Tropomyosin alpha |
| 11 | TPM3—tropomyosin 3 isoform 1 |
| 12 | TPM4—Isoform 1 of Tropomyosin alpha |
| 13 | TPM4—Isoform 2 of Tropomyosin alpha |
| 14 | CAST—calpastatin isoform a |
| 15 | CCL21—C-C motif Chemokine 21 |
| 16 | CSRP3—Cysteine and glycine-rich protein 3 |
| 17 | CYCS—Cytochrome c |
| 18 | DBI—Isoform 2 of Acyl-CoA-binding protein |
| 19 | FST—Isoform 1 of Follistatin |
| 20 | MDH1—Malate dehydrogenase, cytoplasmic |
| 21 | MDH2—Malate dehydrogenase, mitochondrial |
| 22 | VIM—Vimentin |
| 23 | PEBP1—Phosphatidylethanolamine-binding protein 1 |
| 24 | LIPC—Hepatic triacylglycerol lipase |
| 25 | FLNC—Isoform 1 of Filamin-C |
| 26 | LRP1—14 kDa protein |
| 27 | AK1—Adenylate kinase 1 |
| 28 | PGAM2—Phosphoglycerate mutase 2 |
| 29 | PARK7—Protein DJ-1 |
| 30 | SPON1—Spondin-1 |
| 31 | TPI1—Isoform 1 of Triosephosphate isomerase 1 |
| 32 | GOT1—Aspartate aminotransferase, cytoplasmic |
| 33 | LTBP1—latent transforming growth factor beta bind. protein 1 |
| 34 | ITGB1—integrin beta 1 isoform 1A protein |
| 35 | PON3—Serum paraoxonase/lactonase 3 |
| 36 | FLNA—filamin A, alpha isoform 1 |
| 37 | LTF—Growth-inhibiting protein 12 |
| 38 | PF4—Platelet factor 4 |
| 39 | CST3; CST2—Cystatin-C |
| 40 | THBS1-- Thrombospondin-1 |
| 41 | IGF2—insulin-like growth factor 2 isoform 2 |
| 42 | PPBP—Platelet basic protein |

A classification of additional known and novel biomarkers identified using the methods described herein is shown below in Table 1B.

TABLE 1B

| # | | Protein name |
|---|---|---|
| 1 | Known | CRP |
| 2 | markers of | MRP14 |
| 3 | cardiovascular | MPO |
| 4 | injury | Troponin I |
| 5 | | Troponin T |
| 6 | | NT-proBNP |
| 7 | | BNP32 |
| 8 | MRM assay in | ACLP Aortic carboxypeptidase-like protein 1 |
| 9 | place | FHL1 four and a half LIM domains 1 isoform 5 |
| 10 | | MYL3 Myosin light chain 3 |
| 11 | | TPM1 Isoform 4 of Tropomyosin alpha-1 chain |
| 12 | Verified by | ANG Angiogenin |
| 13 | ELISA | CCL21 C-C motif chemokine 21 |
| 14 | | ACBP Isoform 2 of Acyl-CoA-binding protein |
| 15 | New candidates | ITGB1 Isoform Beta-1C of Integrin beta-1 |
| 16 | detected in | CSRP3 Cysteine and glycine-rich protein 3 |
| 17 | first AIMS | FLNC Isoform 1 of Filamin-C |
| 18 | expt | TAGLN Transgelin |
| 19 | | PGAM2 Phosphoglycerate mutase 2 |
| 20 | | GOT1 Aspartate aminotransferase, |
| | | cytoplasmic |
| 21 | | PEBP1 Phosphatidylethanolamine-binding |
| | | protein 1 |
| 22 | | CSRP1 Cysteine and glycine-rich protein 1 |
| 23 | | CAST calpastatin isoform a |
| 24 | | TPM3 tropomyosin 3 isoform 1 |
| 25 | | TPM4 Isoform 1 of Tropomyosin alpha-4 chain |
| 26 | | TPM4 Isoform 2 of Tropomyosin alpha-4 chain |
| 27 | New candidates | FGL2 Fibroleukin |
| 28 | from the new | BASP1 Brain acid soluble protein 1 |
| 29 | AIMS list | MYOC Myocilin |
| 30 | | SCUBE1 Signal peptide, CUB and EGF-like |
| | | domain-containing protein 1 |
| 31 | | FSTL1 Follistatin-related protein 1 |

As shown in Table 1B, several markers of cardiovascular injury are known in the art (e.g., CRP, MRP14, MPO, Troponin I, Troponin T, NT-proBNP, and BNP32). However, many additional biomarkers that have not previously been directly associated with myocardial infarction and/or cardiovascular injury have also been identified using the methods described herein. Moreover, the combination of any two or more biomarkers (or of one or more known markers (e.g., proteins 1-7 shown in Table 1B) with one or more of the novel biomarkers identified herein (e.g., proteins 8-31 shown in Table 1B)) as a biomarker for cardiovascular injury has also never previously been reported.

Thus, detection of one or more of the early cardiovascular biomarkers described herein is diagnostic of cardiovascular injury. Specifically, one or more (preferably two or more) of the biomarkers listed in Table 1A and/or Table 1B can be detected in the practice of the present invention. For example, two (2), three (3), four (4), five (5), ten (10), fifteen (15), twenty (20), forty (40) or more biomarkers can be detected. In some aspects, all biomarkers listed herein can be detected. Preferred ranges from which the number of biomarkers can be detected include ranges bounded by any minimum selected from between one (1) and forty-two (42) (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42).

Those skilled in the art will recognize that any one (or more) of the candidate biomarker proteins identified in accordance with the methods described herein (e.g., the proteins listed in Tables 1A and/or 1B) may be useful (alone or in any combination) as markers of cardiovascular disease and injury.

For example, one potential biomarker that has emerged from the discovery work in the Planned MI samples is Acyl-CoA binding protein (ACBP), a 10 kDa cytoplasmic protein that binds medium- and long-chain fatty acyl-CoA esters and plays a role in fatty acid metabolism. Long-chain fatty acyl-CoA esters function as substrates and intermediates in lipid biosynthesis and catabolism and also play a role in regulating carbohydrate metabolism, protein sorting, gene expression, and signal transduction. Homeostatic control of these molecules is, therefore, essential for numerous cellular functions. Previous work has determined that rapid cardiac-specific changes in ACBP occur in response to Planned MI. It was hypothesized that ACBP would also be a marker of exercise-induced myocardial ischemia in a well phenotyped cohort of individuals undergoing exercise testing.

Plasma levels of ACBP were measured at baseline, peak exercise, and 60-minutes post exercise in 53 subjects with exercise induced ischemia and 53 at-risk controls who were referred for exercise stress testing but were found to not have inducible ischemia. By univariate analysis, baseline levels of ACBP were associated with diabetes as well as creatinine and insulin levels. Baseline ACBP levels were inversely related to LVEF and exercise capacity. However, there was no difference in resting levels of ACBP between subjects with inducible ischemia and controls.

At peak exercise, ACBP levels were 34% higher in patients with inducible ischemia compared to controls (28.5±2.1 vs. 21.3±1.2, P=0.006). In multivariate analysis, peak ACBP levels remained predictive of exercise-induced myocardial ischemia following adjustment for age, gender, and BMI (P=0.029). Peak exercise ACBP also remained predictive of inducible ischemia after adjustment for baseline cardiac risk factors including hypertension, diabetes, hyperlipidemia, tobacco use, and family history of CAD.

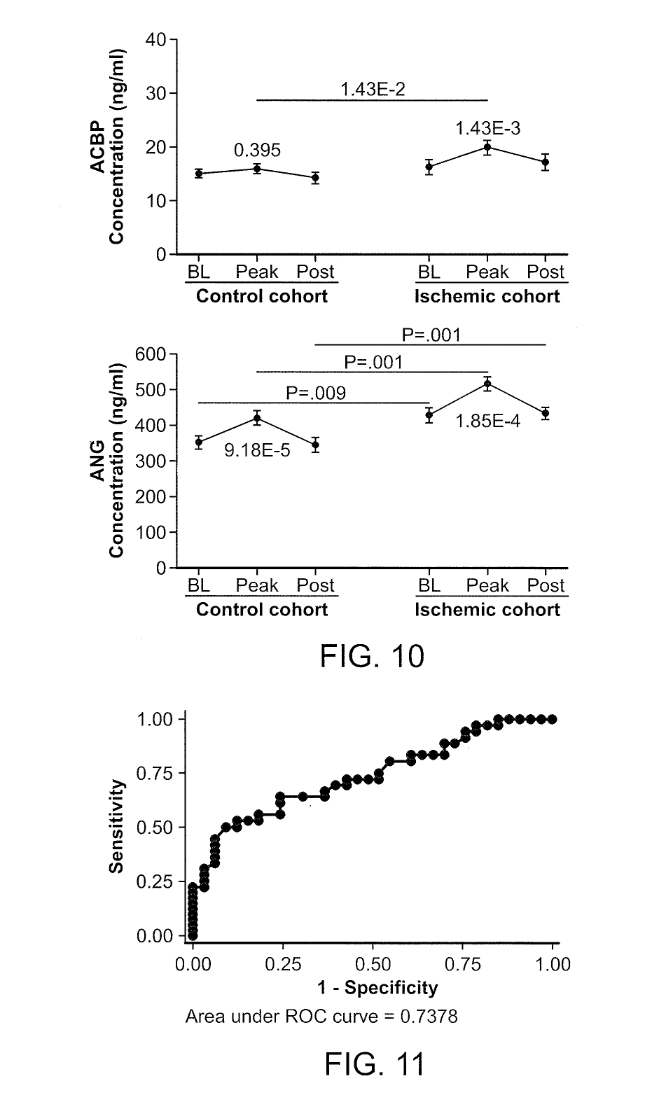

These findings have also been validated in another 50 individuals with exercise induced ischemia and 50 at-risk controls. In the second cohort, ACBP levels at peak exercise were 21% higher in the ischemic individuals (P<0.01). Again, in the new cohort peak, ACBP levels predicted ischemia even after adjustment for all baseline clinical cardiac risk factors (P=0.017). Furthermore, the changes in ACBP levels (peak−baseline) were even more strongly associated with myocardial ischemia (P=0.001). ROC curve analyses confirmed that ACBP levels were a strong predictor of ischemic class (ischemia vs. no ischemia), as seen in FIG. 11.

Finally, a striking association was found between the degree of change in ACBP with exercise and the degree of myocardial ischemia quantified by sestamibi imaging using a four point ischemia score (0=none, 1=mild, 2=mod, 3=severe; P=0.002). This “graded” association adds significant enthusiasm to the interpretation that a novel marker of ischemia has been identified.

### Detecting Biomarkers

In one preferred embodiment, cardiovascular damage and/or injury in a subject is analyzed by (a) providing a biological sample isolated from a subject suspected of having, for example without limitation, an acute myocardial infarction; (b) detecting in the sample the presence or amount of at least one (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or more) of the biomarkers listed in Tables 1A, 1B, and/or 4, fragments or variants thereof; and (c) correlating the presence or amount of the marker with the presence of cardiovascular injury and/or damage in the subject.

Immediately after injury to the cardiovascular system (such as an acute myocardial infarction or other ischemic event), the cardiovascular damage causes an efflux of these biomarker proteins first into the space or biological fluid immediately surrounding the origin or site of injury and eventually into the circulating blood. Obtaining biological fluids such as blood, plasma, or serum from a subject is typically much less invasive and traumatizing than obtaining a tissue biopsy sample. Thus, samples that encompass biological fluids are preferred for use in the invention. Peripheral blood, in particular, is preferred for detecting cardiovascular injury in a subject as it is readily obtainable.

The actual measurement of levels of one or more the novel biomarkers of the invention can be determined at the protein or nucleic acid level using any method(s) known in the art.

These methods include, without limitation, and in particular, PCR methods, including, without limitation, real time PCR, reverse transcriptase PCR and real time reverse transcriptase PCR; sequencing methods, including high-throughput sequencing; nucleic acid chips, mass spectrometry (e.g., laser desorption/ionization mass spectrometry), fluorescence, surface plasmon resonance, ellipsometry and atomic force microscopy. See for example, U.S. Pat. Nos. 5,723,591; 5,801,155 and 6,084,102 and Higuchi, 1992 and 1993. PCR assays may be done, for example, in a multi-well plate formats or in chips, such as the BioTrove OPEN ARRAY Chips (BioTrove, Woburn, Mass.). In one embodiment, levels of expression of the biomarkers of the present invention are detected by real-time PCR, as described further herein.

For example, at the nucleic acid level, Northern and Southern hybridization analysis, as well as ribonuclease protection assays using probes which specifically recognize one or more of these sequences can be used to determine gene expression. Alternatively, levels of biomarkers can be measured using reverse-transcription-based PCR assays (RT-PCR), e.g., using primers specific for the differentially expressed sequence of genes. Levels of biomarkers can also be determined at the protein level, e.g., by measuring the levels of peptides encoded by the gene products described herein, or activities thereof. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the biomarker genes according to the activity of each protein analyzed.

The biomarker proteins, polypeptides, mutations, and polymorphisms thereof can be detected in any suitable manner, but is typically detected by contacting a biological sample from the subject with an antibody which binds the biomarker protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Those skilled in the art will be familiar with numerous specific immunoassay formats and variations thereof any of which may be useful for carrying out the embodiments of the invention disclosed herein.

Using sequence information provided by the database entries for the biomarker sequences, expression of the biomarker sequences can be detected (if present) and measured using techniques well known to one of ordinary skill in the art. For example, sequences within the sequence database entries corresponding to biomarker sequences, or within the sequences disclosed herein, can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like. Preferably, levels of expression of the biomarkers of the present invention is detected by real-time PCR, as described further herein.

The sample from the subject is typically a biological fluid as described above, and may be the same sample of biological fluid used to conduct the method described above.

The methods for detecting these biomarkers in a sample have many applications. For example, one or more biomarkers can be measured to aid cardiovascular injury diagnosis or prognosis. In another example, the methods for detection of the biomarkers can be used to monitor responses in a subject to cardiovascular injury treatment. In another example, the methods for detecting biomarkers can be used to assay for and to identify compounds that modulate expression of these biomarkers in vivo or in vitro.

### Sample Preparation

Nucleic acids may be obtained from the samples in many ways known to one of skill in the art, for example, extraction methods, including e.g., solvent extraction, affinity purification and centrifugation. Selective precipitation can also purify nucleic acids. Chromatography methods may also be utilized including, gel filtration, ion exchange, selective adsorption, or affinity binding. The nucleic acids may be, for example, RNA, DNA or may be synthesized into cDNA. The nucleic acids may be detected using microarray techniques that are well known in the art, for example, Affymetrix arrays followed by multidimensional scaling techniques. (See R. Ekins and F. W. Chu, Microarrays: their origins and applications. Trends Biotechnol., 1999, 17, 217-218; D. D. Shoemaker, et al., Experimental annotation of the human genome using microarray technology, Nature 409(6822): 922-927 (2001) and U.S. Pat. No. 5,750,015.)

In yet another embodiment, a sample can be fractionated using a sequential extraction protocol. In sequential extraction, a sample is exposed to a series of adsorbents to extract different types of biomolecules from a sample. For example, a sample is applied to a first adsorbent to extract certain nucleic acids, and an eluant containing non-adsorbent proteins (i.e., nucleic acids that did not bind to the first adsorbent) is collected. Then, the fraction is exposed to a second adsorbent. This further extracts various nucleic acids from the fraction. This second fraction is then exposed to a third adsorbent, and so on. Any suitable materials and methods can be used to perform sequential extraction of a sample. For example, a series of spin columns comprising different adsorbents can be used. In another example, multi-well plates comprising different adsorbents at its bottom can be used. In another example, sequential extraction can be performed on a probe adapted for use in a gas phase ion spectrometer, wherein the probe surface comprises adsorbents for binding biomolecules. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomolecules in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate two-dimensional array of spots of biomolecules, including one or more biomarkers. See, e.g., Jungblut and Thiede, Mass Spectr. Rev. 16: 145-162 (1997). The two-dimensional gel electrophoresis can be performed using methods known in the art. See, e.g., Deutscher (ed.), Methods Enzymol. vol. 182.

In yet another embodiment, high performance liquid chromatography (HPLC) can be used to separate a mixture of biomolecules in a sample based on their different physical properties, such as polarity, charge and size. HPLC instruments typically consist of a reservoir of mobile phase, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution or to determine its identity. For example, the biomarkers may be subject to proteolytic digestion before analysis to remove contaminating proteins. Any protease known in the art can be used.

Once captured on a substrate, e.g., biochip, any suitable method, such as those described herein as well as other methods known in the art, can be used to measure a biomarker or biomarkers in a sample.

### Use of a Data Analysis Algorithm

Detection of the level of expression of any one or more of the biomarkers described herein can be analyzed using any suitable means known in the art.

In one embodiment of the invention, the number of features that may be used to classify an individual is optimized to allow a classification of an individual with high certainty. For example, comparison of the individual's biomarker profile to a reference biomarker profile comprises applying a decision rule. The decision rule can comprise a data analysis algorithm, such as a computer pattern recognition algorithm. Other suitable algorithms include, but are not limited to, logistic regression or a nonparametric algorithm that detects differences in the distribution of feature values (e.g., a Wilcoxon Signed Rank Test). The decision rule may be based upon one, two, three, four, five, 10, 20 or more features. In one embodiment, the decision rule is based on hundreds or more of features. Applying the decision rule may also comprise using a classification tree algorithm. For example, the reference biomarker profile may comprise at least three features, where the features are predictors in a classification tree algorithm. The data analysis algorithm predicts membership within a population (or class) with an accuracy of at least about 60%, at least about 70%, at least about 80% and at least about 90%.

Suitable algorithms are known in the art, some of which are reviewed in Hastie et al. Such algorithms classify complex spectra from biological materials, such as a blood sample, to distinguish individuals as normal or as possessing biomarker expression levels characteristic of a particular disease state. While such algorithms may be used to increase the speed and efficiency of the application of the decision rule and to avoid investigator bias, one of ordinary skill in the art will realize that computer-based algorithms are not required to carry out the methods of the present invention.

Algorithms may be applied to the comparison of biomarker profiles, regardless of the method that was used to generate the biomarker profile. For example, suitable algorithms can be applied to biomarker profiles generated using gas chromatography, as discussed in Harper, “Pyrolysis and GC in Polymer Analysis,” Dekker, N.Y. (1985). Further, Wagner et al., Anal. Chem. 74: 1824-35 (2002) disclose an algorithm that improves the ability to classify individuals based on spectra obtained by static time-of-flight secondary ion mass spectrometry (TOF-SIMS). Additionally, Bright et al., J. Microbiol. Methods 48: 127-38 (2002) disclose a method of distinguishing between bacterial strains with high certainty (79-89% correct classification rates) by analysis of MALDI-TOF-MS spectra. Dalluge, Fresenius J. Anal. Chem. 366: 701-11 (2000) discusses the use of MALDI-TOF-MS and liquid chromatography-electrospray ionization mass spectrometry (LC/ESI-MS) to classify profiles of biomarkers in complex biological samples.

### Correlation and Data Analysis

The methods for detecting these biomarkers in a sample have many applications. For example, one or more biomarkers can be measured to aid cardiovascular injury diagnosis or prognosis and/or to determine the severity of the cardiovascular injury in the subject. In another example, the methods for detection of the biomarkers can be used to monitor responses in a subject to cardiovascular injury treatment(s). In other examples, the methods for detecting biomarkers can be used to assay for and to identify compounds that modulate expression of these biomarkers in vivo or in vitro.

Detection of biomarkers can be analyzed using any suitable means, including arrays. Nucleic acid arrays may be analyzed using software, for example, Applied Maths, Belgium. GenExplore™: 2-way cluster analysis, principal component analysis, discriminant analysis, self-organizing maps; BioDiscovery, Inc., Los Angeles, Calif. (ImaGene™, special image processing and data extraction software, powered by MatLab®; GeneSight: hierarchical clustering, artificial neural network (SOM), principal component analysis, time series; AutoGene™; CloneTracker™); GeneData AG (Basel, Switzerland); Molecular Pattern Recognition web site at MIT's Whitehead Genome Center; Rosetta Inpharmatics, Kirkland, Wash. Resolver™ Expression Data Analysis System; Scanalytics, Inc., Fairfax, Va. Its MicroArray Suite enables researchers to acquire, visualize, process, and analyze gene expression microarray data; TIGR (The Institute for Genome Research) offers software tools (free for academic institutions) for array analysis. For example, see also Eisen M B, Brown P O., Methods Enzymol. 1999; 303: 179-205.

Those skilled in the art will recognize that the pairing of simple enzyme-linked immunoadsorbent assays (ELISA) can be used for detection and correlation of biomarkers, as these types of assays are most relevant to large populations.

In one embodiment, data generated, for example, by desorption is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of biomarkers detected, including the strength of the signal generated by each biomarker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other biomarkers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling biomarkers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique biomarkers and biomarkers which are up- or down-regulated between samples. Biomarker profiles (spectra) from any two samples may be compared visually. In yet another format, Spotfire Scatter Plot can be used, wherein biomarkers that are detected are plotted as a dot in a plot, wherein one axis of the plot represents the apparent molecular of the biomarkers detected and another axis represents the signal intensity of biomarkers detected. For each sample, biomarkers that are detected and the amount of biomarkers present in the sample can be saved in a computer readable medium. This data can then be compared to a control or reference biomarker profile or reference value (e.g., a profile or quantity of biomarkers detected in control, e.g., subjects in whom cardiovascular injury is undetectable).

When the sample is measured and data is generated, e.g., by mass spectrometry, the data is then analyzed by a computer software program. Generally, the software can comprise code that converts signal from the mass spectrometer into computer readable form. The software also can include code that applies an algorithm to the analysis of the signal to determine whether the signal represents a “peak” in the signal corresponding to a marker of this invention, or other useful biomarkers. The software also can include code that executes an algorithm that compares signal from a test sample to a typical signal characteristic of “normal” and determines the closeness of fit between the two signals. The software also can include code indicating which the test sample is closest, thereby providing a probable diagnosis.

In preferred methods of the present invention, multiple biomarkers are measured. The use of multiple biomarkers increases the predictive value of the test and provides greater utility in diagnosis, toxicology, subject stratification and subject monitoring. The process called “Pattern recognition” detects the patterns formed by multiple biomarkers greatly improves the sensitivity and specificity of clinical proteomics for predictive medicine. Subtle variations in data from clinical samples indicate that certain patterns of protein expression can predict phenotypes such as the presence or absence of a certain disease, a particular stage of disease progression, or a positive or adverse response to drug treatments.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety. In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines). A preferred supervised classification method is a recursive partitioning process.

Recursive partitioning processes use recursive partitioning trees to classify spectra derived from unknown samples. In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, International Application No. WO 01/31580 (Barnhill et al., “Methods and devices for identifying patterns in biological systems and methods of use thereof,” May 3, 2001); U.S. Patent Application No. 2002/0193950 A1 (Gavin et al., “Method or analyzing mass spectra,” Dec. 19, 2002); U.S. Patent Application No. 2003/0004402 A1 (Hitt et al., “Process for discriminating between biological states based on hidden patterns from biological data,” Jan. 2, 2003); and U.S. Patent Application No. 2003/0055615 A1 (Zhang and Zhang, “Systems and methods for processing biological expression data” Mar. 20, 2003).

More specifically, to obtain the biomarkers the peak intensity data of samples from subjects, e.g., cardiovascular injury subjects, and healthy controls are used as a “discovery set.” These data were combined and randomly divided into a training set and a test set to construct and test multivariate predictive models using a non-linear version of Unified Maximum Separability Analysis (“USMA”) classifiers. Details of USMA classifiers are described in U.S. Patent Application No. 2003/0055615. The invention provides methods for aiding a cardiovascular injury diagnosis using one or more biomarkers as specified herein. These biomarkers can be used alone, in combination with other biomarkers in any set, or with entirely different biomarkers in aiding human cardiovascular injury diagnosis. For example, the biomarkers of the current invention are expressed at an elevated level and/or are present at a higher frequency in subjects with cardiovascular injury when compared with normal subjects. Therefore, detection of one or more of these biomarkers in a person would provide useful information regarding the probability that the person may have cardiovascular injury.

In any of the methods disclosed herein, the data from the sample may be fed directly from the detection means into a computer containing the diagnostic algorithm. Alternatively, the data obtained can be fed manually, or via an automated means, into a separate computer that contains the diagnostic algorithm. Accordingly, embodiments of the invention include methods involving correlating the detection of the biomarker or biomarkers with a probable diagnosis of cardiovascular injury. The correlation may take into account the amount of the biomarker or biomarkers in the sample compared to a control amount of the biomarker or biomarkers (up or down regulation of the biomarker or biomarkers) (e.g., in normal subjects). The correlation may take into account the presence or absence of the biomarkers in a test sample and the frequency of detection of the same biomarkers in a control. The correlation may take into account both of such factors to facilitate determination of whether a subject has a cardiovascular injury or not.

The measurement of biomarkers can involve quantifying the biomarkers to correlate the detection of biomarkers with a probable diagnosis of cardiovascular injury. Thus, if the amount of the biomarkers detected in a subject being tested is elevated compared to a control amount, then the subject being tested has a higher probability of having cardiovascular injury.

The correlation may take into account the amount of the biomarker or biomarkers in the sample compared to a control amount of the biomarker or biomarkers (up or down regulation of the biomarker or biomarkers) (e.g., in normal subjects). A control can be, e.g., the average or median amount of biomarker present in comparable samples of normal subjects in normal subjects. The control amount is measured under the same or substantially similar experimental conditions as in measuring the test amount. The correlation may take into account the presence or absence of the biomarkers in a test sample and the frequency of detection of the same biomarkers in a control. The correlation may take into account both of such factors to facilitate diagnosis.

In certain embodiments, the methods further comprise managing subject treatment based on the status. As before the management of the subject describes the actions of the physician or clinician subsequent to diagnosis of cardiovascular injury. For example, if the result of the methods of the present invention is inconclusive or there is reason that confirmation of status is necessary, the physician may order more tests (e.g., CT scans, PET scans, MRI scans, PET-CT scans, X-rays, biopsies, blood tests (LFTs, LDH). Alternatively, if the status indicates that treatment is appropriate, the physician may schedule the subject for treatment. In other instances, the subject may receive therapeutic treatments, either in lieu of, or in addition to, surgery. No further action may be warranted. Furthermore, if the results show that treatment has been successful, a maintenance therapy or no further management may be necessary.

The invention also provides for such methods where the biomarkers (or specific combinations of biomarkers) are measured again after subject management. In these cases, the methods are used to monitor the, response to treatment. Because of the ease of use of the methods and the lack of invasiveness of the methods, the methods can be repeated (i.e., on a periodic basis) after each treatment the subject receives. This allows the physician to follow the effectiveness of the course of treatment. If the results show that the treatment is not effective, the course of treatment can be altered accordingly. This enables the physician to be flexible in the treatment options.

In another example, the methods for detecting biomarkers can be used to assay for and to identify compounds that modulate expression or activity of these biomarkers in vivo or in vitro.

The methods of the present invention have other applications as well. For example, the biomarkers can be used to screen for compounds that modulate the expression of the biomarkers in vitro or in vivo, which compounds in turn may be useful in treating or preventing cardiovascular injury in subjects. In another example, the biomarkers can be used to monitor the response to treatments for cardiovascular injury.

In a preferred embodiment of the invention, a diagnosis based on the presence or absence in a test subject of any the biomarkers of this invention is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

A dataset can be analyzed by multiple classification algorithms. Some classification algorithms provide discrete rules for classification; others provide probability estimates of a certain outcome (class). In the latter case, the decision (diagnosis) is made based on the class with the highest probability. Other classification algorithms and formulae include, but are not limited to, Principal Component Analysis (PCA), cross-correlation, factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, Leave-One-Out (LOO), 10-Fold cross-validation (10-Fold CV), and Hidden Markov Models, among others.

### Antibodies

As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments which lack the Fc fragment of intact antibody, clear more rapidly from the circulation, and may have less non-specific tissue binding of an intact antibody (Wahl et al., (1983) J. Nucl. Med. 24:316-325. The antibodies of the invention comprise whole native antibodies, bispecific antibodies; chimeric antibodies; Fab, Fab′, single chain V region fragments (scFv) and fusion polypeptides.

“Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Biomarkers and Methods of the Invention

The invention also includes cardiovascular injury candidate genes, which are useful as therapeutic targets. These genes include, for example, those listed herein.

The methods of the present invention have other applications as well. For example, the biomarkers can be used to screen for compounds that modulate the expression of the biomarkers in vitro or in vivo, which compounds in turn may be useful in treating or preventing cardiovascular injury in subjects. In another example, the biomarkers can be used to monitor the response to treatments for cardiovascular injury.

Thus, for example, the kits of this invention could include a solid substrate having a hydrophobic function, such as a protein biochip (e.g., a Ciphergen ProteinChip array), to detect the product of the nucleic acid biomarkers, and a buffer for washing the substrate, as well as instructions providing a protocol to measure the biomarkers of this invention on the chip and to use these measurements to diagnose cardiovascular injury. Methods for identifying a candidate compound for treating cardiovascular injury may comprise, for example, contacting one or more of the protein products of the biomarkers of the invention with a test compound; and determining whether the test compound interacts with the protein, wherein a compound that interacts with the protein is identified as a candidate compound for treating cardiovascular injury. Compounds suitable for therapeutic testing may be screened initially by identifying compounds which interact with one or more of the proteins that are the products of the biomarkers identified herein. By way of example, screening might include recombinantly expressing a protein, purifying the protein, and affixing the protein to a substrate. Test compounds would then be contacted with the substrate, typically in aqueous conditions, and interactions between the test compound and the protein are measured, for example, by measuring elution rates as a function of salt concentration. Certain proteins may recognize and cleave one or more proteins of this invention, in which case the proteins may be detected by monitoring the digestion of one or more proteins in a standard assay, e.g., by gel electrophoresis of the proteins.

In a related embodiment, the ability of a test compound to inhibit the activity of one or more of the proteins of this invention may be measured. One of skill in the art will recognize that the techniques used to measure the activity of a particular protein will vary depending on the function and properties of the protein. For example, an enzymatic activity of a protein may be assayed provided that an appropriate substrate is available and provided that the concentration of the substrate or the appearance of the reaction product is readily measurable. The ability of potentially therapeutic test compounds to inhibit or enhance the activity of a given protein may be determined by measuring the rates of catalysis in the presence or absence of the test compounds. The ability of a test compound to interfere with a non-enzymatic (e.g., structural) function or activity of one of the protein of this invention may also be measured. For example, the self-assembly of a multi-protein complex which includes one of the proteins of this invention may be monitored by spectroscopy in the presence or absence of a test compound. Alternatively, if the protein is a non-enzymatic enhancer of transcription, test compounds which interfere with the ability of the protein to enhance transcription may be identified by measuring the levels of protein-dependent transcription in vivo or in vitro in the presence and absence of the test compound.

Test compounds capable of modulating the activity of any of the proteins may be administered to subjects who are suffering from or are at risk of developing cardiovascular injury. For example, the administration of a test compound which decreases the activity of a particular protein may decrease the risk from cardiovascular injury in a subject if the increased activity of the protein is responsible, at least in part, for the onset of cardiovascular injury.

In a related embodiment, the ability of a test compound to inhibit the gene expression of one or more of the biomarkers of this invention may be measured. One of skill in the art will recognize that the techniques used to measure the levels of a particular can be applied to a sample and test compounds can be evaluated for the ability to reduce the level of expression of the biomarker.

At the clinical level, screening a test compound includes obtaining samples from test subjects before and after the subjects have been exposed to a test compound. The CNA levels in the samples of one or more of the biomarkers of this invention may be measured and analyzed to determine whether the levels of the biomarkers change after exposure to a test compound. The samples may be analyzed by PCR, as described herein, or the samples may be analyzed by any appropriate means known to one of skill in the art. In a further embodiment, the changes in the level of expression of one or more of the biomarkers may be measured using in vitro methods and materials. For example, human cultured cells which express, or are capable of expressing, one or more of the biomarkers of this invention may be contacted with test compounds. Subjects who have been treated with test compounds will be routinely examined for any physiological effects which may result from the treatment. As one embodiment, the test compounds will be evaluated for their ability to decrease disease likelihood in a subject. Alternatively, if the test compounds are administered to subjects who have previously been diagnosed with cardiovascular injury, test compounds will be screened for their ability to slow or stop the progression of the disease.

### Kits

The invention also provides kits that are useful in detecting a cardiovascular injury or event in an individual, wherein the kit can be used to detect one or more of the cardiovascular injury biomarkers described herein. Preferably, the kits of the present invention comprise at least one cardiovascular injury-specific biomarker. Specific biomarkers that are useful in the present invention are set forth herein. The biomarkers of the kit can be used to generate biomarker profiles according to the present invention. Examples of classes of compounds of the kit include, but are not limited to, proteins, and fragments thereof, peptides, polypeptides, proteoglycans, glycoproteins, lipoproteins, carbohydrates, lipids, nucleic acids, organic and inorganic chemicals, and natural and synthetic polymers. The biomarker(s) may be part of an array, or the biomarker(s) may be packaged separately and/or individually. The kit may also comprise at least one internal standard to be used in generating the biomarker profiles of the present invention. Likewise, the internal standards can be any of the classes of compounds described above. The kits of the present invention also may contain reagents that can be used to detectably label biomarkers contained in the biological samples from which the biomarker profiles are generated. For this purpose, the kit may comprise a set of antibodies or functional fragments thereof that specifically bind at least two, three, four, five, ten, twenty, thirty, forty or more of the biomarkers set forth in Tables 1A, 1B, and/or 4. The antibodies themselves may be detectably labeled. The kit also may comprise a specific biomarker binding component, such as an aptamer. If the biomarkers comprise a nucleic acid, the kit may provide an oligonucleotide probe that is capable of forming a duplex with the biomarker or with a complementary strand of a biomarker. The oligonucleotide probe may be detectably labeled.

For example, the kits can be used to detect any one or more of the cardiovascular injury biomarkers described herein, which are differentially present in samples of cardiovascular injury subjects and normal subjects. The kits of the invention have many applications. For example, the kits can be used in any one of the methods of the invention described herein, such as, inter alia, to differentiate if a subject has cardiovascular injury, thus aiding a diagnosis. In another example, the kits can be used to identify compounds that modulate expression of one or more of the biomarkers in in vitro or in vivo animal models.

Generally, kits of the present invention include a biomarker-detection reagent, e.g., nucleic acids that specifically identify one or more biomarker nucleic acids by having homologous nucleic acid sequences, such as oligonucleotide sequences complementary to a portion of the biomarker nucleic acids. The oligonucleotides can be fragments of the biomarker genes. The oligonucleotides may be single stranded or double stranded. For example the oligonucleotides can be 200, 150, 100, 50, 25, 10 or less nucleotides in length. The kit may contain in separate containers a nucleic acid (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay and for correlation may be included in the kit.

For example, biomarker detection reagents can be immobilized on a solid matrix such as a porous strip to form at least one biomarker detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of biomarkers present in the sample. The detection sites may be configured in any suitably detectable shape and are typically in the shape of a bar or dot spanning the width of a test strip.

Alternatively, the kit contains a nucleic acid substrate array comprising one or more nucleic acid sequences, e.g., primers for nucleic acid amplification. The nucleic acids on the array specifically identify one or more nucleic acid sequences represented by the biomarkers of the present invention. In various embodiments, the expression of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or more (i.e., all) of the sequences represented by the biomarkers described herein can be identified by virtue of binding to the array. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.). The kit may also contain reagents, and/or enzymes for amplifying or isolating sample DNA. The kits may include reagents for real-time PCR, for example, TaqMan probes and/or primers, and enzymes.

In one embodiment, a kit comprises: (a) a substrate comprising an adsorbent thereon, wherein the adsorbent retains or is otherwise suitable for binding a biomarker, and (b) instructions to detect the biomarker or biomarkers by contacting a sample with the adsorbent and detecting the biomarker or biomarkers retained by the adsorbent. In some embodiments, the kit may comprise an eluant (as an alternative or in combination with instructions) or instructions for making an eluant, wherein the combination of the adsorbent and the eluant allows detection of the biomarkers using gas phase ion spectrometry. Such kits can be prepared from the materials described above, and the previous discussion of these materials (e.g., probe substrates, adsorbents, washing solutions, etc.) is fully applicable to this section.

In another embodiment, the kit may comprise a first substrate comprising an adsorbent thereon (e.g., a particle functionalized with an adsorbent) and a second substrate onto which the first substrate can be positioned to form a probe, which is removably insertable into a gas phase ion spectrometer. In other embodiments, the kit may comprise a single substrate, which is in the form of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

Optionally, the kit may further comprise pre-fractionation spin columns. In some embodiments, the kit may further comprise instructions for suitable operation parameters in the form of a label or a separate insert. Optionally, the kit may further comprise a standard or control information so that the test sample can be compared with the control information standard to determine if the test amount of a biomarker detected in a sample is a diagnostic amount consistent with a diagnosis of cardiovascular injury.

The kits of the present invention may also include pharmaceutical excipients, diluents and/or adjuvants when the biomarker is to be used to raise an antibody. Examples of pharmaceutical adjuvants include, but are not limited to, preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of an injectable pharmaceutical form can be brought about by the inclusion of agents which delay absorption such as aluminum monostearate and gelatin.

### EXAMPLES

### Example 1

### Planned Myocardial Injury

Three studies were initiated that take advantage of instances where cardiac injury is controlled in the hospital setting. The first is a study of planned myocardial infarction, which occurs in patients undergoing alcohol septal ablation for hypertrophic cardiomyopathy, a recently adopted treatment to relieve the outflow tract obstruction by causing a controlled myocardial infarction of the offending muscle of the interventricular septum. (See Lakkis et al., Circulation 98:1750-55 (1998)) In this “controlled” or “planned” myocardial infarction (PMI), alcohol is injected into the first septal branch of the left anterior descending artery. This causes endothelial damage, thrombosis, and myocardial infarction with septal thinning, and subsequent amelioration of the impingement on left ventricular outflow. The second is a study of planned myocardial ischemia, in which patients who were referred for catheterization for stable exertional angina underwent rapid atrial pacing in an attempt to induce ischemia in those with coronary artery stenoses. The third is also a study of planned myocardial ischemia, which patients with significant coronary artery disease experience when undergoing an exercise stress test.

Each of these studies offers a unique window into otherwise spontaneous pathological processes. Blood samples can be obtained at multiple time points after the perturbation, allowing for the carefully controlled study of the kinetics of release of any proteins from the injured heart and an assessment of a range of injury from transient ischemia to frank infarction. A critical advantage is that blood can be obtained just prior to and following the procedure. This allows each patient to serve as his or her own baseline control and markedly simplifies data analysis. In addition, as the pacing procedure and PMI is performed in the cardiac catheterization suite, “proximal fluids” can be obtained via coronary sinus sampling. By obtaining blood directly from the cardiac venous system, proteins released from the heart are naturally enriched potentially up to 25- to 50-fold. Not only does coronary sinus sampling concentrate a subset of the proteins of interest, it also sheds insight into the anatomical source of the observed proteins. In samples which are simultaneously drawn from the coronary sinus vs. the periphery, proteins produced by the heart will be more abundant in the coronary sample; proteins present at equal concentrations in the coronary sinus and in the periphery are generated by other organs. While available markers are proteins that are released from the myocardium by necrosis or apoptosis, unbiased approaches might identify sensitive markers or response mediators elaborated by other organs. Preliminary results from these studies are described in Example 2, infra.

### Example 2

### Planned Myocardial Infarction (PMI) Recapitulates Spontaneous Myocardial Infarction

The study described herein demonstrates integration of modern mass spectrometers and proteomic technologies into a discovery-through-verification biomarker pipeline that yields novel cardiovascular biomarkers meriting further evaluation in large, heterogeneous patient cohorts.

A proteomics-based biomarker discovery-through-verification pipeline was used to identify early biomarkers of cardiovascular injury from blood samples of patients undergoing a therapeutic, “planned” myocardial infarction (“PMI”), a septal ablation for hypertrophic cardiomyopathy (see Sigwart et al., Lancet 346:211-214 (1995); Knight et al., Circulation 95; 2075-81 (1997)) that faithfully reproduces spontaneous MI (see Lakkis et al., Circulation 98:1750-55 (1998); Lakkis et al., J. Am. Coll. Cardiol. 36:852-55 (2000)) In this procedure, blood is serially sampled directly from the heart before and after controlled myocardial injury allowing each patient to serve as their own biological control.

LC-MS/MS analyses detected 121 highly differentially expressed proteins across discovery patients, including previously credentialed markers of cardiovascular disease and many potentially novel biomarkers. After qualification with accurate inclusion mass screening (AIMS), a subset of novel candidates were measured in peripheral plasma of patients with PMI or spontaneous MI and controls using quantitative, multiple reaction monitoring MS-based assays or immunoassays, and were shown to be specific to MI.

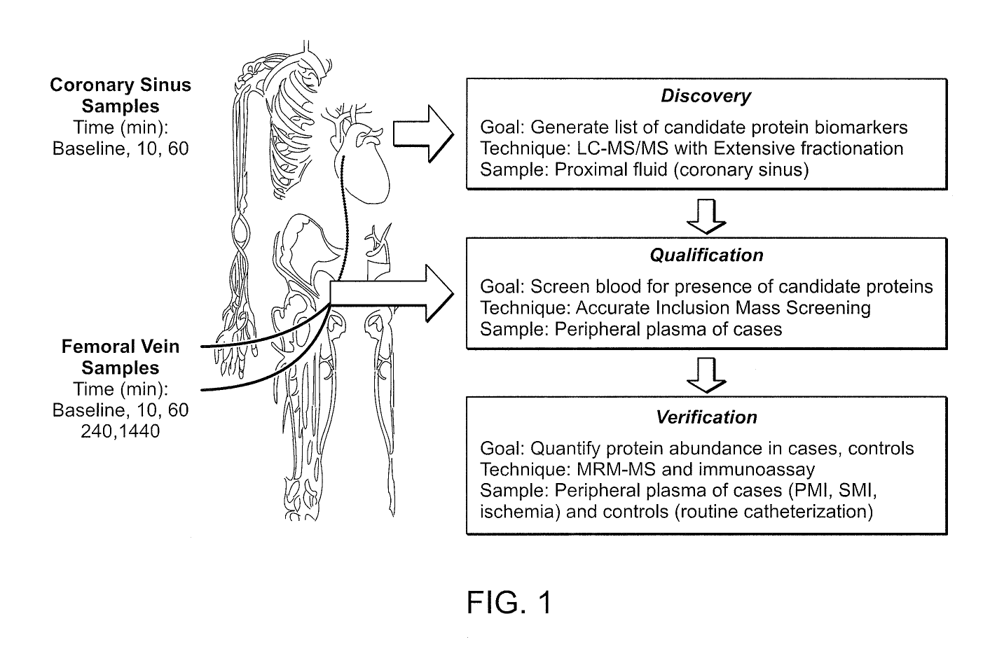

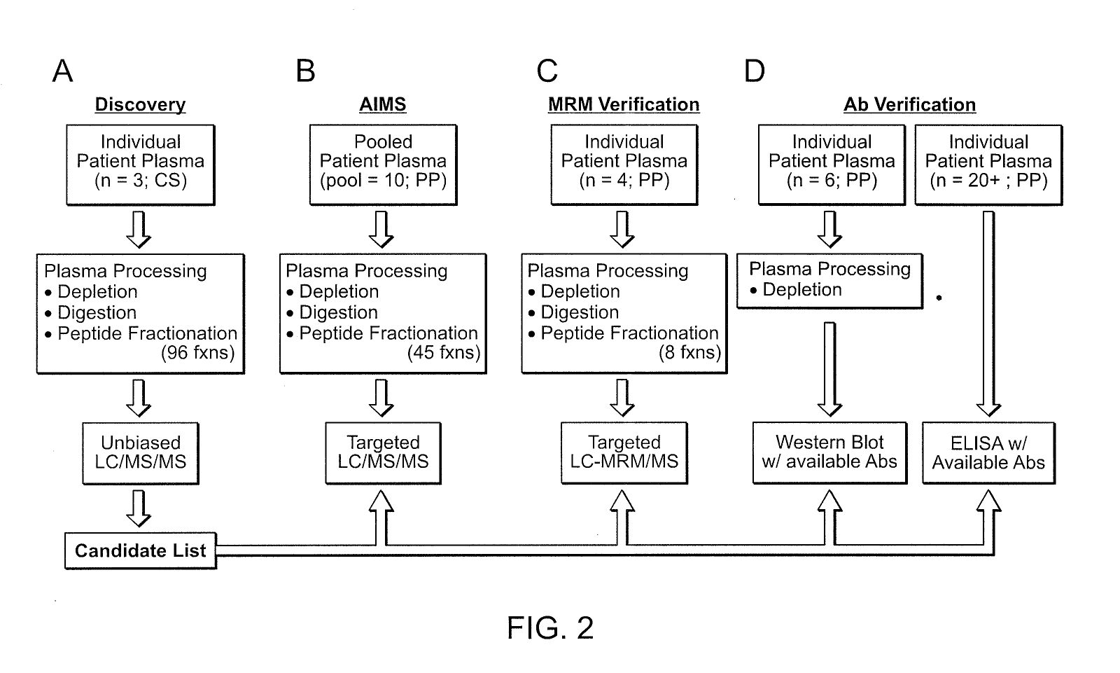

An overview of the biomarker pipeline and its application to a human model of myocardial injury is shown in FIG. 1. An overview of the sample preparation workflow for discovery proteomics (A), qualification by AIMS (B), targeted, quantitative assays by MRM/MS (C), and verification by Western blot analysis and ELISA assays with available antibodies (D) is shown in FIG. 2. Workflow (A) represents the methods used for discovery proteomics whereby CS from individual patients was immunoaffinity depleted, enzymatically digested and the subsequent peptides separated extensively prior to unbiased LC/MS/MS. Workflow (B) represents the methods used for AIMS whereby peripheral plasma from a pool of 10 PMI patients was immunoaffinity depleted, enzymatically digested and the subsequent peptides moderately separated prior to targeted LC/MS/MS. Workflow (C) represents the methods used for targeted MRM assays whereby peripheral plasma from individual PMI patients was immunoaffinity depleted, enzymatically digested and subsequent peptides separated by limited fractionation prior to targeted, quantitative assays by MRM/MS. Workflow (D) represents the methods used for Ab verification whereby CS was immunoaffinity depleted prior to Western blot analysis and peripheral plasma from patients was analyzed directly by immunoassay.

### Methods

### 1. Clinical Cohorts for Discovery and Blood Collection:

1.1. Planned MI Cohort (Patients with Hypertrophic Obstructive Cardiomyopathy (HOCM) Undergoing Septal Abalation.

The study described herein began with a planned myocardial infarction (PMI) model to give the highest likelihood of finding changes in the setting of a large myocardial insult. Patients undergoing planned MI using alcohol septal ablation for the treatment of symptomatic hypertrophic obstructive cardiomyopathy (HOCM) were included in the study. The PMI cohort consisted of 22 patients with HOCM. Inclusion criteria for this cohort were: 1) primary HOCM; 2) septal thickness of 16 mm or greater; 3) resting outflow tract gradient of greater than 30 mmHg, or an inducible outflow tract gradient of at least 50 mm Hg; 4) symptoms refractory to optimal medical therapy; and 5) appropriate coronary anatomy.

The most proximal accessible septal branch was instrumented using standard angioplasty guiding catheters and guidewires and 1.5 or 2.0 mm×9 mm Maverick™ balloon catheters. Radiographic and echocardiographic contrast injections confirmed proper selection of the septal branch and balloon catheter position. Ethanol was infused through the balloon catheter at 1 ml per minute. Additional injections in the same or other septal branches were administered as needed, causing cessation of blood flow to the isolated myocardium, and to reduce the gradient to <20 mmHg (See Baggish et al., Heart 92:1773-78 (2006)) Blood was drawn at baseline (just prior to the onset of the ablation) and at 10 minutes, 1 hour, 2 hours, 4 hours, and 24 hours following the onset of injury. Of the 22 patients, 11 consented to the placement of a catheter to the coronary sinus during the ablation, allowing for the simultaneous sampling of blood from the coronary sinus and femoral catheters at baseline, 10 minutes, and 60 minutes. The coronary sinus catheter was subsequently removed prior to the patient leaving the catheterization suite.

1.2 Patients Undergoing Elective Cardiac Catheterization.

A cohort of 24 patients undergoing elective, diagnostic cardiac catheterization for cardiovascular disease, but not acute myocardial ischemia, were recruited as controls for the PMI patients and spontaneous MI patients. Blood was drawn prior to the onset of cardiac catheterization and at 10 minutes and 1 hour after the procedure was begun.

1.3 Exercise Tolerance Testing (ETT) Cohort (Patients Undergoing Cardiac Stress Testing).

The ETT cohort provides consisted of patients who underwent stress testing using the standard Bruce protocol (see Baggish et al., Heart 92:1773-78 (2006)) with myocardial perfusion imaging at Brigham and Women's Hospital or Massachusetts General Hospital. One hundred and eleven patients were referred to the MGH Exercise laboratory for bicycle ergometry cardiopulmonary exercise testing. Symptoms, heart rate, blood pressure, and a 12-lead ECG were recorded before the test, midway through each stage, and during recovery. The stress test was terminated if there was physical exhaustion, severe angina, >2 mm horizontal or downsloping ST-segment depression, ≧20 mm Hg fall in systolic blood pressure, or sustained ventricular arrhythmia. Duration of the stress test, metabolic equivalents (METs) achieved, peak heart rate, and peak blood pressure were recorded. If the patient developed angina during the test, the timing, quality (typical vs. atypical), and effect on the test (limiting or non-limiting) were noted. The maximal horizontal or downsloping ST segment changes were recorded in each ECG lead.

A stress-rest imaging protocol was used. 99Tc tetrofosmin was administered at peak stress and imaging was performed soon thereafter. Four hours later, a second injection was administered and repeat imaging was performed. Quantitative analysis of perfusion was performed using the CEqual method to calculate the percent reversible and fixed perfusion defects. (See Knight et al., Circulation 95:2075-81 (1997)) Patients with >5% reversible perfusion defect were selected as cases (53 Patients) and those without any perfusion defect were selected as controls. Left ventricular ejection fraction was calculated using commercially available software. (See Horiba et al., Circulation 114:1713-20 (2006)) Blood samples were obtained just prior to the test (baseline, exhausted or positive EKG/image appearing during the test (peak) and fully recovered after the test (post).

1.4 Patients with Spontaneous ACS.

This cohort consisted of 23 patients with spontaneous acute coronary syndrome. These patients were undergoing emergent cardiac catheterization for acute ST-segment elevation, spontaneous MI within 8 hours of symptom onset. For this cohort, blood samples were obtained in the coronary catheterization suite.

All blood samples were collected in K₂EDTA-treated tubes (Becton Dickinson) and were centrifuged at 2000×g for 10 minutes to pellet cellular elements. The supernatant plasma was then aliquoted and immediately frozen at −80° C. Additional blood samples were sent to the clinical chemistry laboratory for evaluation of the standard cardiac markers creatine kinase (CK), CK-MB, and Troponin T (Roche Diagnostics).

### 2. Sample Preparation for Discovery Proteomics Studies

2.1 Protein Depletion and Enzymatic Digestion for Discovery Proteomics.

Coronary sinus plasma from 3 patients collected at baseline and 10 minutes and 60 minutes post ablation was immunoaffinity depleted of twelve high abundance proteins using an IgY-12 high capacity LC10 column (12.7×79 mm; GenWay Biotech, San Diego, Calif.) according to manufacturer's instructions. Depleted plasma was concentrated to the original starting volume via Vivaspin 15R concentrators (5000 molecular weight cutoff, Vivascience, Hannover, Germany). Protein concentrations of depleted, concentrated plasma were performed by Coomassie Plus Bradford assay (Pierce, Rockford, Ill.).

500 μg of depleted CS plasma per time point per patient was denatured with 6M Urea, 10 mM Tris, pH 8.0, reduced with 20 mM dithiothreitol at 37° C. for 30 minutes, and alkylated with 50 mM iodoacetamide at room temperature in the dark for 30 minutes. Urea concentration was diluted to 2M with water prior to a 4 hour digestion with LysC (Wako, Richmond, Va.) at 1:50 (w/w) enzyme to substrate ratio at 37° C. Urea was further diluted with water to 0.6M prior to overnight digestion at 37° C. with trypsin (sequencing grade modified, Promega, Madison, Wis.) using a 1:50 w/w enzyme to substrate ratio. Digests were terminated with formic acid to a final concentration of 1% and desalted using Oasis HLB 3 cc (60 mg) reversed phase cartridges (Waters, Milford, Mass.) as described previously. (See Keshishian et al., Mol Cell Proteomics 6:2212-29 (2007)) Eluates were frozen, dried to dryness via vacuum centrifugation, and stored at −80° C.

2.2 Strong Cation Exchange Chromatography (SCX) for Discovery Proteomics.

Digested plasma samples from each patient and time point were normalized to 500 ug total protein. Samples were reconstituted in 75 μl of 25% acetonitrile, pH3.0, and fractionated using a BioBasic 1×250 mm column (ThermoFisher, San Jose, Calif.) on an Agilent 1100 capillary LC system (Agilent Technologies, Palo Alto, Calif.) at a flow rate of 20 μl/min Mobile phase consisted of 25% acetonitrile, pH3.0 (A) and 250 mM ammonium formate in 25% acetonitrile, pH3.0 (B). After loading the sample onto the column, the mobile phase was held at 3% B for 10 minutes, and peptides were separated with a linear gradient of 3-100% B in 120 minutes. Fractions were collected every 1.25 minutes for a total of 96 fractions collected, 80 of which were subsequently analyzed by nanoLC/MS/MS (see below). All fractions were dried to dryness by vacuum centrifugation and stored at −80° C. until mass spectrometric analysis.

2.3 nanoLC/MS/MS for Discovery Proteomics.

For protein identification, each of the 80 SCX fractions was resconstituted in 7 μl of 5% formic acid/3% acetonitrile and analyzed on an LTQ-Orbitrap FT mass spectrometer (Thermo-Fishier Scientific) coupled to an Agilent 1100 nano-LC system (Agilent Technologies, Palo Alto, Calif.). Chromatography was performed using a 15-cm column (Picofrit 10 μm ID, New Objectives) packed in-house with ReproSil-Pur C18-AQ 3 μm reversed phase resin (Dr. Maisch, GmbH). The mobile phase consisted of 0.1% formic acid as solvent A and 90% acetonitrile, 0.1% formic acid as solvent B. Peptides were eluted at 200 nL/min with a gradient of 3-7% B for 2 min, 7-37% B in 90 min, 37-90% B in 10 min, and 90% B for 9 min. A single Orbitrap MS scan from m/z 300-1800 was followed by up to eight ion trap MS/MS scans on the top 8 most abundant precursor ions. Dynamic exclusion was enabled with a repeat count of 2, a repeat duration of 20 sec, and exclusion duration of 20 sec. MS/MS spectra were collected with normalized collision energy of 28 and an isolation width of 3 amu.

2.4 Protein Identification for Discovery Proteomics.

All discovery data was processed using Agilent Spectrum Mill MS Proteomics Workbench (Agilent Technologies, Palo Alto, Calif.). MS/MS spectra were searched against the human International Protein Index (IPI) database (version 3.48) with parent mass tolerance of 20 ppm, fragment mass tolerance 0.7 Da, a maximum of two missed cleavages, and carbamidomethylation and oxidized methionines/pyroglutamic acid as fixed and variable modifications, respectively. Database matches for individual spectra were autovalidated according to user-defined scoring thresholds for both peptides and proteins in a two step process. For protein autovalidation (step 1), autovalidation criteria included a cumulative score of ≧25 based upon individual scores of multiple peptides derived from a given protein. Peptide scores in protein mode had to be ≧10 with a scored peak intensity (SPI) of ≧70% for peptides with a precursor charge state of +2. Scored peak intensity refers to the percentage of the annotated MS/MS spectrum that is explained by the database match. Peptides with precursor charges of +3 and +4 had to meet scoring thresholds of ≧13 and 70% SPI. For peptide autovalidation (step 2), single peptides derived from a given protein had to meet scoring thresholds of ≧13 and ≧70% SPI for all charge states. In both autovalidation steps, the delta rank1-rank2 threshold was >2.

In Spectrum Mill, false discovery rates (FDRs) are calculated at 3 different levels: spectrum, distinct peptide, and distinct protein. Peptide FDRs are calculated in Spectrum Mill using essentially the same pseudo-reversal strategy evaluated by Elias and Gygi (see Elias et al, Nat. Methods 4:207-214 (2007)), and shown to perform the same as concatenation. A false distinct protein ID occurs when all of the distinct peptides which group together to constitute a distinct protein have a deltaForwardReverseScore ≦0. The settings were adjusted to provide peptide FDR of <1%. Spectrum Mill also carries out protein grouping using the methods described by Nesvizhskii and Aebersold (see; Neshvizhskii et al. Mol Cell Proteomics 4:1419-40 (2005))

### 3. Sample Preparation for Accurate Inclusion Mass Screening (AIMS)

3.1 Protein Depletion and Enzymatic Digestion for AIMS.

25 uL of peripheral plasma from 10 PMI patients collected at baseline and 10 min and 60 min post ablation was pooled prior to depletion for a total of 3 samples for AIMS analysis. Patient plasma was immunoaffinity depleted of fourteen high abundance proteins using a Multiple Affinity Removal System (10 mm×100 mm; Agilent Technologies) according to manufacturer's instructions. Depleted plasma was concentrated to the original starting volume and buffer exchanged with 50 mM ammonium bicarbonate via Amicon Ultra-4 (3000 molecular weight cutoff, Millipore, Billerica, Mass.). Protein concentrations of depleted, concentrated plasma were determined by BCA assay (Thermo Fisher Scientific, Rockford, Ill.).

Depleted and concentrated peripheral plasma was denatured with 6M Urea, reduced with 20 mM dithiothreitol at 37° C. for 30 minutes, and alkylated with 50 mM iodoacetamide at room temperature in the dark for 30 minutes. Urea concentration was diluted to 2M with 50 mM ammonium bicarbonate prior to a 4 hour digestion with LysC (Wako, Richmond, Va.) at 1:50 (w/w) enzyme to substrate ratio at 37° C. Urea was further diluted with 50 mM ammonium bicarbonate to 0.6M prior to overnight digestion at 37° C. with trypsin (sequencing grade modified, Promega, Madison, Wis.) using a 1:50 w/w enzyme to substrate ratio. Digests were terminated with formic acid to a final concentration of 1% and desalted using Oasis HLB 1 cc (30 mg) reversed phase cartridges (Waters, Milford, Mass.) as described previously. (See Keshishian et al., Mol Cell Proteomics 6:2212-29 (2007)) Eluates were frozen, dried to dryness via vacuum centrifugation, and stored at −80° C.

3.2 Strong Cation Exchange Chromatography (SCX) for AIMS.

Digested plasma samples from each pooled time point were normalized to 400 ug total protein for SCX fractionation. Digests were separated using a Polysulfoetyl A 2.1×200 mm column on an Agilent 1100 analytical LC system, and mobile phase A of 10 mM ammonium formate in 25% acetonitrile, pH 3.0, and mobile phase B of 500 mM ammonium formate in 25% acetonitrile, pH 6.8. Samples were reconstituted in mobile phase A and peptides were fractionated at a flow rate of 200 μL/min with a gradient of 1-50% B for 40 min, 50-100% B for 10 min, and a hold at 100% B for 10 min. Fractions were collected based upon volume as follows: 290 μl fractions for the first 32 min, followed by 100 μl fractions from 32 to 36 min, 65 μl fractions from 36 to 46 min, 100 μl fractions from 46 to 54 min, and 305 μl fractions from 54 to 100 min Pooling of fractions to a total of 45 fractions for mass spectrometric analysis was based on the complexity of each fraction. One to three fractions were pooled together for a total of 37 fractions from 32 to 65 min of the gradient, 3 fractions were pooled from 9 to 32 min, and 4 fractions were pooled from 65 to 100 min The latter fractions were desalted using Oasis 1 cc (10 mg) cartridges (Waters, Milford, Mass.) as described previously. (See Keshishian et al., Mol Cell Proteomics 6:2212-29 (2007)) All of the fractions were dried to dryness by vacuum centrifugation and were stored at −80° C.

3.3 nanoLC/MS/MS for AIMS.

For protein identification, each of the 45 SCX fractions was reconstituted in 12 μl of 5% formic acid/3% acetonitrile and 2 μl of it was analyzed on an LTQ-Orbitrap FT mass spectrometer (Thermo-Fishier Scientific) coupled to an Agilent 1100 nano-LC system (Agilent Technologies, Palo Alto, Calif.). Chromatography was performed using a 15-cm column (Picofrit 10 μm ID, New Objectives) packed in-house with ReproSil-Pur C18-AQ 3 μm reversed phase resin (Dr. Maisch, GmbH). The mobile phase consisted of 0.1% formic acid as solvent A and 90% acetonitrile in 0.1% formic acid as solvent B. Peptides were eluted at 200 nL/min with a gradient of 3-7% B for 2 min, 7-37% B in 90 min, 37-90% B in 10 min, and 90% B for 9 min. An inclusion list of 1152 entries representing the m/z, z pairs of 982 peptides derived from 82 proteins was used with a precursor mass tolerance of +/−5 ppm. A single Orbitrap MS scan from m/z 300 to 1500 was followed by up to five ion trap MS/MS scans. The top five most abundant precursors from the inclusion list (if present) were targeted for MS/MS spectrum acquisition over the course of the experiment. Preview mode and charge state screening were enabled for selection of precursors. The m/z tolerance around targeted precursors was +/−5 ppm and lock mass was not enabled. Dynamic exclusion was enabled with a repeat count of 2 and exclusion duration of 15 sec. MS/MS spectra were collected with normalized collision energy of 28, an isolation width of 2.5 amu, and activation time of 30 ms.

3.4 Protein Identification for AIMS.

All MS/MS spectra acquired from AIMS experiments were searched against the human IPI database (version 3.60) with parent mass tolerance of 15 ppm, fragment mass tolerance of 0.7 Da, two missed cleavages, and carbamidomethylation as a fixed modification. Thresholds used for autovalidation included peptide scores of ≧13 with a scored peak intensity of ≧70% and a cumulative protein score of ≧25.

### 4. Sample Preparation for Stable Isotope Dilution Multiple Reaction Monitoring (SID-MRM)

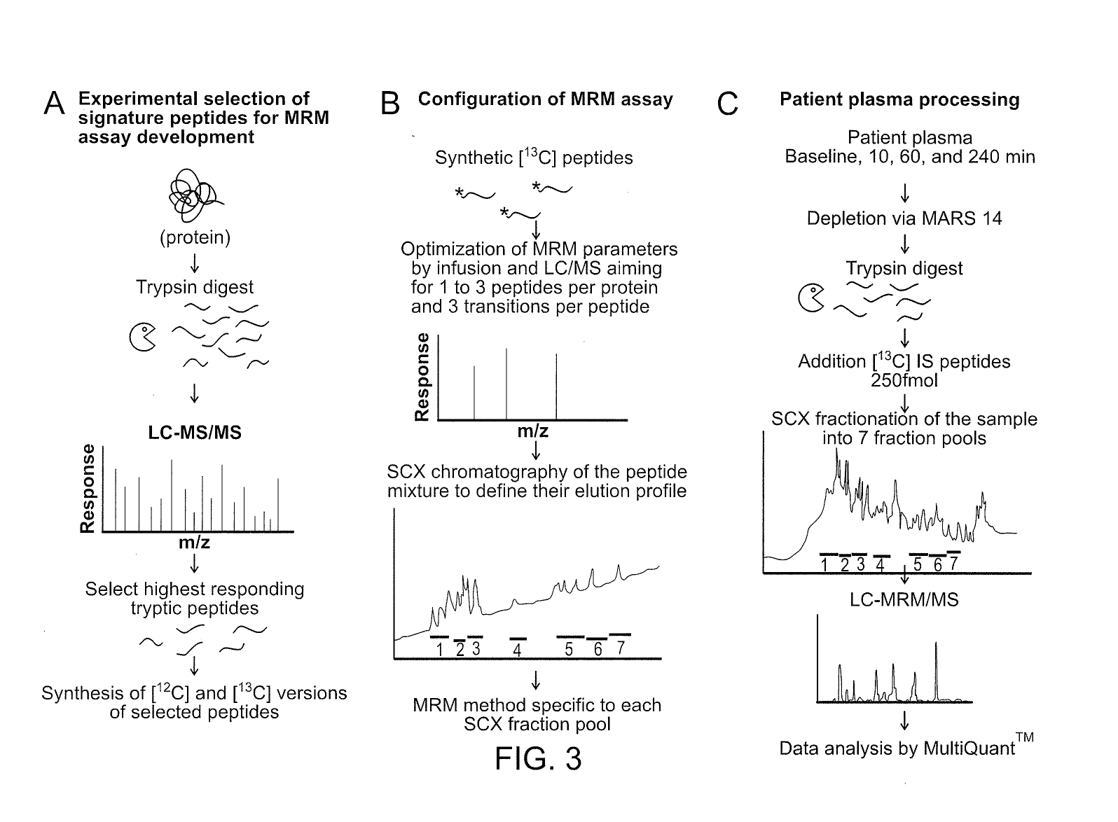

An overview of assay configuration and sample preparation for SID-MRM experiments is shown in FIG. 3.

4.1 Labeled Peptide Internal Standards.

Table 2 lists the protein targets and their “signature peptides” for which final MRM assays were configured. Signature peptides have both high responses in electrospray LC-MS/MS, and are sequence unique when searched against a non-redundant human protein database (NCBInr). Signature peptides were selected based upon observed peptides in the discovery data as well as peptides that were computationally predicted to have high response by electrospray MS. (See Fusaro et al., Nat Biotechnol 27:190-98 (2009))

Thirteen peptides derived from AEBP1, MYL3, and FHL1 proteins were synthesized with a single, uniformly labeled [¹³C₆]Lysine or [¹³C₆]Arginine on the C-terminus by 21^{st} Century Biochemicals (Marlboro, Mass.). Two peptides derived from Tropomyosin 1 were synthesized with a single, uniformly labeled [¹³C₆,¹⁵N₂]Lysine or [¹³C₆,¹⁵N₄]Arginine on the C-terminus by Thermo Fisher Scientific (Rockford, Ill.). Unlabeled [¹²C] forms of each peptide were also synthesized by 21st Century Biochemicals (Marlboro, Mass.). Synthetic peptides were purified to >90% purity and analyzed by amino acid analysis (AAA Service Laboratory Inc, Damascus, Oreg.). Calculations of concentration were based upon the amino acid analysis.

4.2 Protein Depletion and Enzymatic Digestion for SID-MRM.

Peripheral plasma from 4 PMI patients collected at baseline and 10 min, 60 min, and 240 min post ablation was immunoaffinity depleted of fourteen high abundance proteins using a Multiple Affinity Removal System (10 mm×100 mm; Agilent Technologies) according to manufacturer's instructions. Depleted plasma was concentrated to the original starting volume and buffer exchanged to 6M Urea/50 mM Tris pH 8.0 via Amicon Ultra-4 (3000 molecular weight cutoff, Millipore, Billerica, Mass.). Protein concentrations of depleted, concentrated plasma were determined by BCA assay (Thermo Fisher Scientific, Rockford, Ill.). Three process replicates per time point per patient were performed for MRM experiments.

100 μL of depleted plasma per time point per patient was reduced with 20 mM dithiothreitol at 37° C. for 30 minutes, and alkylated with 50 mM iodoacetamide at room temperature in the dark for 30 minutes. Urea concentration was diluted to 2M with water prior to a 2 hour digestion with LysC (Wako, Richmond, Va.) at 1:50 (w/w) enzyme to substrate ratio at 37° C. Urea was further diluted with water to 0.6M prior to overnight digestion at 37° C. with trypsin (sequencing grade modified, Promega, Madison, Wis.) using a 1:50 w/w enzyme to substrate ratio. Digests were terminated with formic acid to a final concentration of 1% and desalted using Oasis HLB 1 cc (30 mg) reversed phase cartridges (Waters, Milford, Mass.) as described previously. (See Keshishian et al., Mol Cell Proteomics 6:2212-29 (2007)) Eluates were frozen, dried to dryness via vacuum centrifugation, and stored at −80° C.

4.3 Strong Cation Exchange Chromatography (SCX) for SID-MRM.

Digested samples were reconstituted in 5 mM potassium phosphate in 25% acetonitrile, pH 3.0 (SCX buffer A) and 250 fmol each of heavy labeled internal standard peptides was added. Separations were performed using a Biobasic 2.1×200 mm column on an Agilent 1100 analytical LC system at a flow rate of 200 μL/min Mobile phases consisted of 5 mM potassium phosphate in 25% acetonitrile, pH 3.0 (A) and 500 mM potassium chloride in 5 mM potassium phosphate in 25% acetonitrile, ph 3.0 (B). After loading the sample onto the column, the mobile phase was held at 1% B for 15 minutes. Peptides were separated with a linear gradient of 1-22% B in 42 minutes, 22-60% B in 2 minutes, and 60-100% B in 2 minutes. Fractions were collected every minute, and acetonitrile removed from collected fractions by vacuum centrifugation. The elution profile of the peptide internal standards was pre-defined and used to generate 8 pools of SCX fractions for MRM analysis per patient per time point. Each pool was desalted using Oasis HLB 1 cc (10 mg) reversed phase cartridges as described previously (see Keshishian et al., Mol Cell Proteomics 6:2212-29 (2007)) and stored at −80° C. until LC-MRM/MS analysis.

4.4 nanoLC-SID/MRM/MS:

Pooled SCX fractions were reconstituted in 30 μL of 5% formic acid/3% acetonitrile. NanoLC-MRM/MS/MS was performed on a QTrap 5500 hybrid triple quadrupole/linear ion trap mass spectrometer (AB Sciex, Foster City, Calif.) coupled to a Eksigent NanoLC-Ultra 2Dplus system (Eksigent, Dublin, Calif.). Chromatography was performed with Solvent A (0.1% formic acid) and Solvent B (90% acetonitrile in 0.1% formic acid). Each sample was injected with full loop injection of 1 μL on PicoFrit columns (75 μm ID, 10 μm ID tip opening, New Objective, Woburn, Mass.) packed in house with 11-12 cm of ReproSil-Pur C18-AQ 3 μm reversed phase resin (Dr. Maisch, GmbH). Sample was eluted at 300 nL/min with a gradient of 3-10% solvent B for 3 min, 10-50% solvent B for 35 min, and 50-90% solvent B for 2 min Data was acquired with an ion spray voltage of 2200V, curtain gas of 20 psi, nebulizer gas of 5 psi, and an interface heater temperature of 150° C. Declustering potential (DP) of 100 and collision cell exit potential (CXP) of 25 was used for all of the transitions. Collision energy (CE) was optimized for maximum transmission and sensitivity of each MRM transition by LC-MRM/MS of a mixture of peptide internal standards and MRMPilot™ 2.0 (AB Sciex, Foster City, Calif.). Identical DP, CE and CXP values were used for each ¹²C/¹³C pair. LC-MRM/MS data acquisition was done by scheduled MRM (sMRM) methods specific to each SCX fraction pool. MRM detection window of 180 second and cycle time of 1 second was used for sMRM. Three MRM transitions per peptide (Table 2) were monitored and acquired at unit resolution both in the first and third quadrupoles (Q1 and Q3) to maximize specificity. In general, transitions were chosen based upon relative abundance and mass-to-charge ratio (m/z) greater than the precursor m/z in the full scan MS/MS spectrum recorded on the QTrap 5500 mass spectrometer. The final MRM method included 162 optimized MRMs for 9 target proteins. These MRMs were distributed among 8 SCX fractions in accordance with the elution profile of the synthetic peptides.

4.5 MRM Data Analysis—

Data analysis was performed using MultiQuant™ software (AB Sciex, Foster City, Calif.). The relative ratios of the three transitions selected and optimized for the final MRM assay were predefined in the absence of plasma (i.e. in buffer) for each peptide using the [¹³C,¹⁵N] internal standards. The most abundant transition for each pair was used for quantification unless interference in this channel was observed.

[¹²C]/[¹³C] peak area ratios were used to calculate concentrations of target proteins in plasma. Coefficient of variation (CV) for each measurement was based on the calculated average protein concentration for a set of 3 process replicates.

### 5.0 Statistical Analyses for Discovery Proteomics

For label-free, relative quantification, the sum of the precursor-ion signal intensities of all peptides derived from each protein was used as an approximation of that protein's expression level across time points, as described below. A minimum detectable 5-fold change was employed between baseline and either 10 min or 60 min samples. This was based on preliminary calculations summarized Table 7. Derivation of Table 7 was based on the t-test, and assumed that the measurements are normally distributed (or can achieve a normal distribution after log transformation), with the CV fixed irrespective of the magnitude of the measurement (i.e., a very conservative CV was used). Furthermore, the significance level is an indicator of the probability that a specific biomarker is a false positive when it has a fold-change larger than the minimum noted in the table. This is therefore a nominal p-value, and does not correct for multiple testing to account for the many hundreds of markers that were evaluated. The table was generated to provide ballpark estimates for minimum detectable fold change, and statistical power attainable for a chosen fold-change level (specifically 3-fold and 5-fold). A staged approach was then used to credential markers, and assess for any false positives that may have been introduced by the process as detailed in the results.

These power calculations suggested that there would be ˜60-80% power to detect changes of five-fold or greater, based on having 6-8 sample pairs (respectively), a nominal significance level of 0.05, and a conservative coefficient of variation of 50% for discovery proteomic findings. There were effectively 6 sample pairs when selecting protein candidates that had a five-fold average change over the combined 10- and 60-minute samples, compared to baseline. For independently detecting changes in the 10- or 60-minute samples, this power will be rapidly attained as more samples are analyzed.

Extracted Ion Chromatograms (XICs)—

The peak area for the XIC of each precursor ion in the intervening high-resolution MS1 scans of the data-dependent LC-MS/MS runs was calculated automatically by the Spectrum Mill software using narrow windows around each individual member of the isotope cluster. Peak widths in both the time and m/z domains are dynamically determined based on MS scan resolution, precursor charge and m/z subject to quality metrics on the relative distribution of the peaks in the isotope cluster vs. theoretical.

### 6.0 Antibody Verification of Candidate Biomarkers

6.1 Western Blot Analysis.

The following commercial antibodies were purchased for Western blot analysis of depleted, peripheral plasma from PMI patients: goat anti-human pleiotrophin (Abcam, Cambridge, Mass.), rabbit anti-human midkine (Antigenix, Huntington Station, N.Y.), mouse anti-human MDH1 (Novus Biological, Littleton, Colo.) and rabbit anti-human ACLP1 (Affinity BioReagents, Goden, Colo.). Depleted peripheral plasma protein was mixed with 6× protein loading buffer and boiled to denature proteins completely, then loaded onto 10% SDS-PAGE gels. SDS gels were then placed into transfer buffer (25 mM Tris, 192 mMglycine, 20% v/v methanol, pH 8.3) for 5 min and the separated proteins were transferred onto nitrocellulose filters. The filter was blocked with 5% nonfat milk powder in TBST (0.05% Tween-20) for 1 h, probed with goat anti-human pleiotrophin (0.1 ug/ml), rabbit anti-human midkine (0.2 ug/ml), mouse anti-human MDH1 (1:500 dilution) or rabbit anti-human ACLP1 (0.2 ug/ml) respectively at 4° C. overnight and incubated with secondary antibody horse radish peroxidase (HRP) labeled anti-rabbit (1:3,000), anti-goat (1:5000) or anti-mouse (1:3000) respectively for 1 hour. The signal was detected by enhanced chemiluminescence (ECL) detection reagents (Amersham, Life Science, Arlington Heights, Ill.).

6.2 ELISA Detection.

Peripheral plasma concentrations of CCL21 (human CCL21/6CKine immunoassay, R&D, Minneapolis, Minn.), angiogenin (human angiogenin ELISA kit, Cell Sciences, Canton, Mass.) and ACBP (human diazepam binding inhibitor ELISA kit, Young In Frontier Co., Seoul, Korea) were measured with commercially available kits according to manufacturer's instructions.

6.3 Statistical Analyses for Clinical Data and ELISA Findings:

For clinical characteristics, values for continuous variables are presented as mean±SD, and comparisons between groups were performed using two-sample t-tests. Association between categorical variables was assessed using the Fisher's Exact Test. To evaluate whether metabolic changes observed in the PMI patients were generalizable to spontaneous MI, proteins for which ELISAs were available that displayed significant changes from baseline at 1, 2 and 4 hours in the derivation and validation planned MI cohorts (P<0.05 at all three time points) were studied. A Wilcoxon Rank-Sum test was used to examine levels of these individual proteins in the patients presenting with spontaneous MI as compared to control patients presenting to the cardiac catheterization suite with non-acute cardiovascular disease.

### Results

Planned MI (PMI) recapitulates spontaneous MI. Clinical characteristics of the PMI patients, as well as the control and validation cohorts are detailed in Table 3. The septal ablation recapitulated important clinical features of spontaneous MI, including substernal chest pain and electrocardiographic changes, as well as the development of echocardiographic evidence of septal wall motion abnormalities, as previously described by the present inventors and others. (See Addona et al., Nat. Biotechnol. 27:633-41 (2009); Keshisian et al., Mol Cell Proteomics 8:1339-2349 (2009)) The standard biochemical metrics of myocardial injury, CK-MB and troponin T, were within normal limits prior to septal ablation and increased to 200±98 ng/ml and 4.5±2.6 ng/ml, respectively. CK-MB peaked at 6.2±2.2 hours and cardiac troponin T at 12±7.6 hours after planned MI, time courses consistent with spontaneous MI. (See Zimmerman et al., Circulation 99:1671-77 (1999))

Discovery of Candidate Biomarkers in the Coronary Sinus (CS) of PMI Patients.

An overview of the proteomics biomarker pipeline and its application to the model of acute myocardial infarction is shown in FIG. 1. A candidate biomarker list was generated in the discovery phase using blood from the CS of three PMI patients sampled at baseline, as well as at 10 minutes and 60 minutes post injury (9 samples total). Plasma was immunoaffinity-depleted of twelve high abundance proteins, enzymatically digested with LysC followed by trypsin, and then extensively fractionated at the peptide level by strong cation exchange (SCX) chromatography into 80 fractions that were analyzed by nanoflow LC-MS/MS. This processing strategy was designed to decrease the dynamic range and complexity of the peptide mixtures analyzed by MS, and thereby maximize detection of lower abundance proteins (see Methods). The MS/MS spectra acquired were searched against the human IPI database using Spectrum Mill Proteomics Workbench.

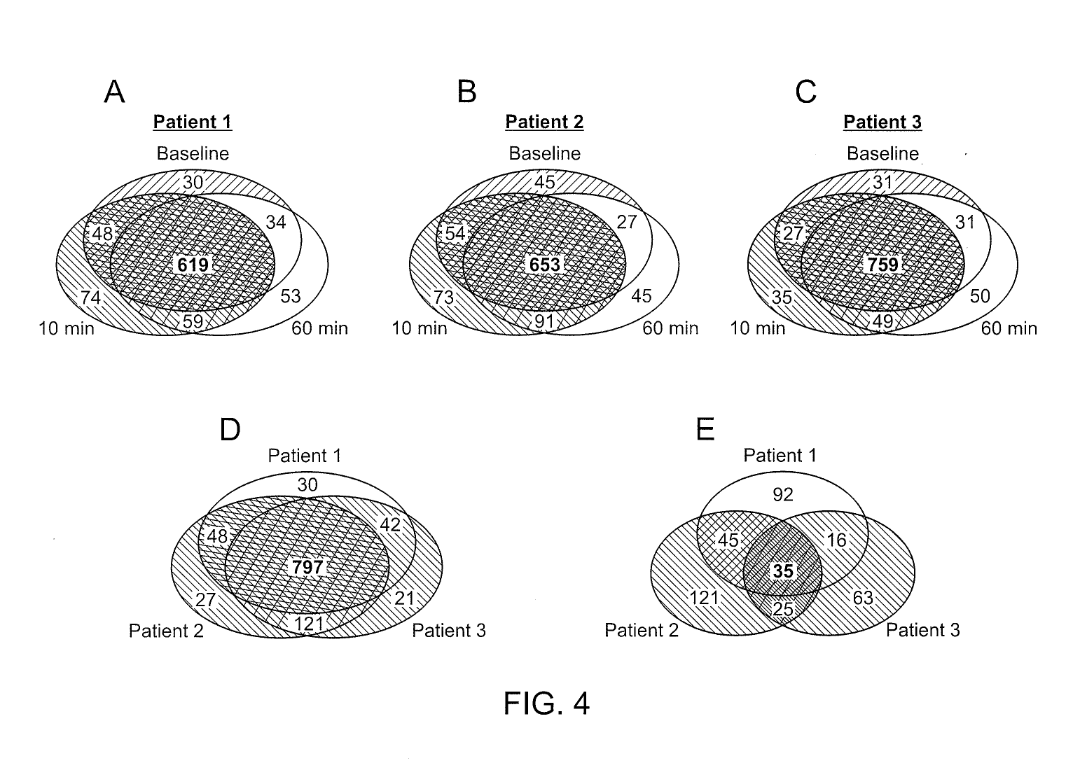

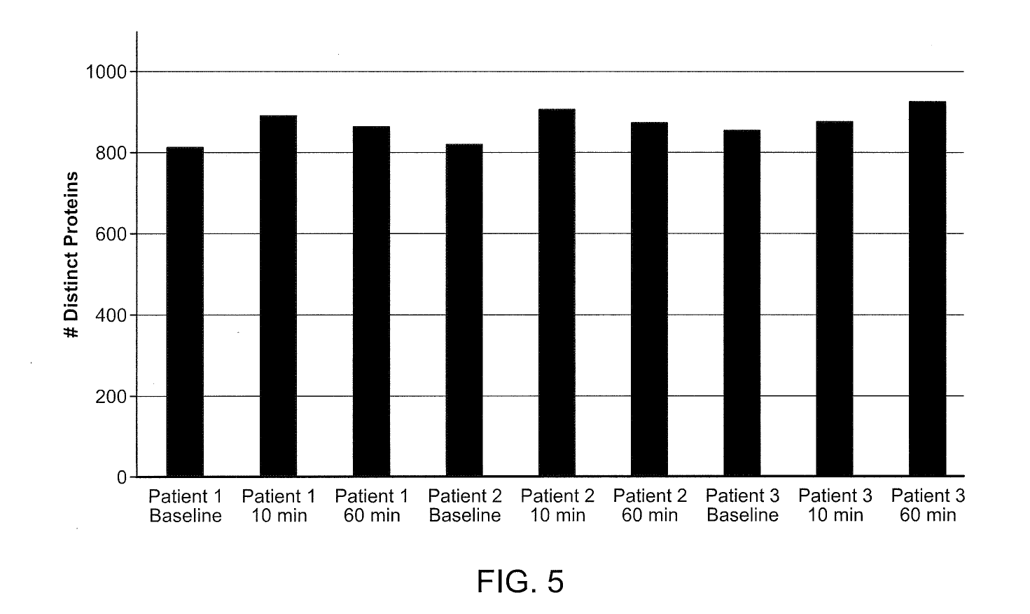

A total of 1086 unique proteins were identified in the nine coronary sinus plasma samples, with an average of 872 proteins/sample using a minimum of two peptides/protein and a peptide false discovery rate (FDR) of ≦2% (FIG. 4). The number of distinct proteins identified in each patient and time point is shown in FIG. 5. Greater than 70% of the proteins identified were observed in all 3 PMI patients (FIG. 4d).

Label-free, relative quantification of peptides (see Methods) was used to identify proteins changing in abundance in the discovery data and to generate a list of candidate biomarker proteins of PMI for subsequent qualification and verification (FIG. 1). Criteria for nomination as a candidate biomarker from the discovery experiments include a minimum of five-fold change in the MS-derived abundance for a minimum of two unique peptides/proteins between baseline and either the 10 minute or 60 minute samples (see Table 7).

A subset of the proteins that met these criteria is presented in Table 4. The finalized list also includes proteins manually selected for biological relevancy. The entire list of 82 proteins (including known markers of myocardial injury) was subsequently analyzed by

Accurate Inclusion Mass Screening for analytical qualification using an independent pool of peripheral plasma collected from 10 PMI patients at baseline and 10 minutes and 60 minutes post ablation. Proteins listed in Table 4 represent those with peptides on the AIMS inclusion list (see Methods). As shown in Table 4, n.d.=not detected. Antibody reagents were commercially available for a minority of the novel candidate biomarkers. These reagents were used to either detect the presence of the protein in plasma by Western or to quantify it by ELISA. The Abs that were tested are as follows: 1=single Ab for Western; 2=two discrete Abs for construction of ELISA; 3=ELISA kit. As shown in Table 4, 35 proteins were increased ≧5-fold as compared to baseline in all three patients at either or both the 10 minute or 60 minute time points, while 86 proteins were increased ≧5-fold in common between any two patients (FIG. 4e).

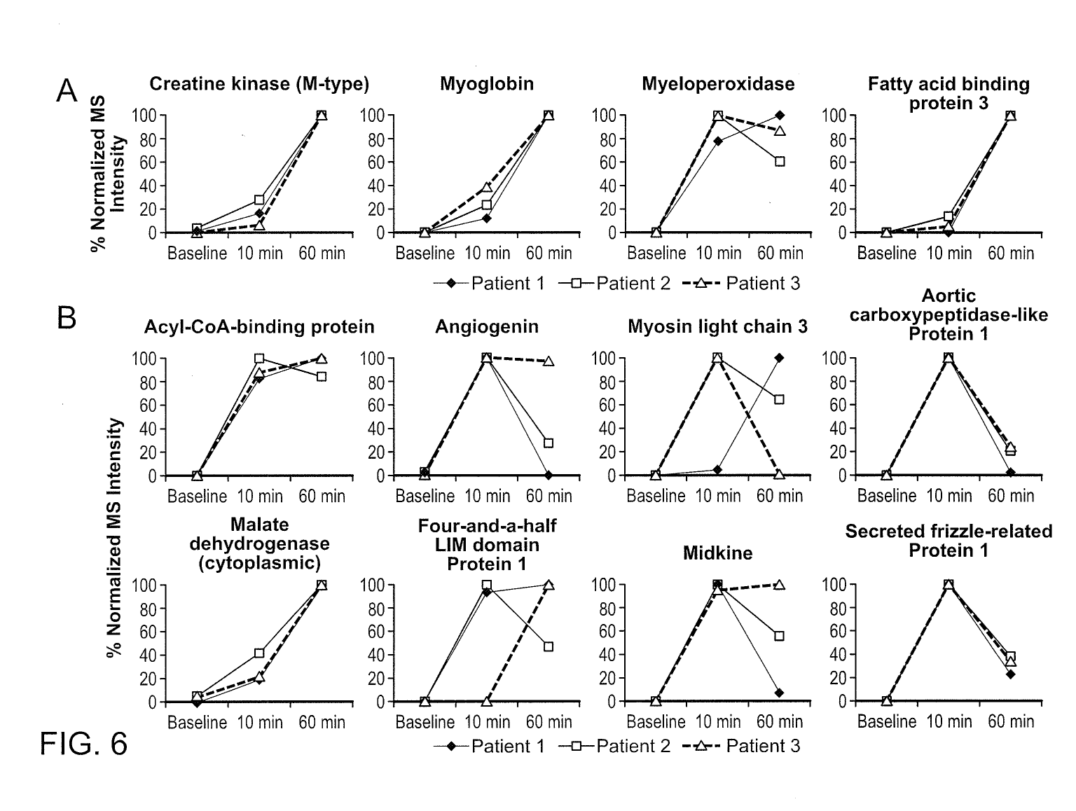

The list of 121 differentially regulated proteins detected in the coronary sinus plasma samples from multiple PMI patients contains many known markers of myocardial injury including myoglobin (MYO), myeloperoxidase (MPO), creatine kinase-myocardial isoform B (CKB), creatine kinase-myocardial isoform M (CKM), and fatty-acid binding protein (FABP). (See de Lemos et al., J. Am. Coll. Cardiol. 40:238-44 (2002); O'Donoghue et al., Circulation 114:550-57 (2006)) Cardiac troponin T (cTnT) was also observed in the discovery data in 2 patients although only a single high scoring peptide of this low abundance protein was detected. The list also contains many potentially novel biomarkers of cardiovascular disease, including aortic carboxypeptidase-like protein (ACLP1), a transcriptional repressor implicated in cardiovascular wound healing (see Layne et al., Mol. Cell. Biol. 21:5256-61 (2001)); four-and-a-half LIM domain protein 1 (FHL1), a cardiomyocyte protein that mediates a hypertrophic biomechanical stress response (see Sheikh et al., J. Clin. Invest. 118:3870-80 (2008)); angiogenin (ANG), a potent mediator of new blood vessel formation (see Kishimoto et al., Oncogene 24:445-56 (2005)); and (MYL3), the regulatory light chain of myosin that may serve as a target for caspase-3 in dying cardiomyocytes (see Moretti et al., Proc. Natl. Acad Sci 99:11860-65 (2002)). Kinetic analyses of the discovery mass spectrometry data for the known (FIG. 6a) and putative biomarkers (FIG. 6b) revealed that these proteins were at very low to undetectable levels in the CS at baseline, then increased by more than 5-fold at 10 and 60 minutes post-PMI in each of the three patients. Almost all of the mass spectrometry changes documented at 10 minutes were also observed at 60 minutes, underscoring the consistency of the findings herein.

Qualification of Candidate Proteins in Peripheral Plasma of PMI Patients by Accurate Inclusion Mass Screening (AIMS).

AIMS technology (see Jaffe et al., Mol. Cell. Proteomics 7:1952-62 (2008)) was incorporated into the pipeline to next ascertain which of the proteins discovered in proximal fluid (e.g., CS plasma) could also be detected in peripheral blood samples from a distinct set of subjects. This step is referred to herein as “Qualification”. AIMS is a targeted MS approach in which MS/MS spectra are triggered and acquired only when an accurate mass and charge pair on the inclusion list are detected. Not only can AIMS be used as an initial qualification step, but prior studies have documented that AIMS also identifies specific peptides that are likely to be well-suited for developing quantitative SID-MRM-MS assays (see Jaffe et al., Mol. Cell. Proteomics 7:1952-62 (2008)), thereby facilitating this resource-intensive activity (see below).

A set of 82 candidate biomarker proteins identified in the CS were qualified by AIMS in three discrete pools of peripheral plasma from 10 patients, each taken at baseline and 10 min and 60 min post ablation from an alternate set of PMI patient samples. The list of proteins for qualification was supplemented with proteins of known relevance to MI, such as cardiac troponin T that was detected in CS discovery experiments, but with only a single high scoring peptide. Several non-specific inflammatory response proteins, as well as heat shock proteins, were eliminated from the prioritization process. Peptides derived from the prioritized list of proteins observed in the discovery data were supplemented with tryptic peptides unique to each candidate protein that were computationally predicted to have high response by electrospray MS (“signature peptides”, see Fusaro et al., Nat. Biotechnol. 27:190-98 (2009)), though not observed in the discovery data set. For these studies, there were 1152 entries on the inclusion list representing the precursor mass and charge pairs for 982 peptides (some in more than one charge state) derived from 82 prioritized candidate proteins selected for qualification. Proteins prioritized for AIMS were selected based upon a minimum of a 5-fold difference in MS abundance between baseline and either 10 minutes or 60 minutes post ablation. Plasma processing for analysis by AIMS was similar to plasma processing for the discovery phase (See FIG. 2). However, it was possible to reduce the number of SCX peptide fractions and therefore the MS data acquisition time by half due to the increased sensitivity of AIMS relative to data-dependent LC-MS/MS.

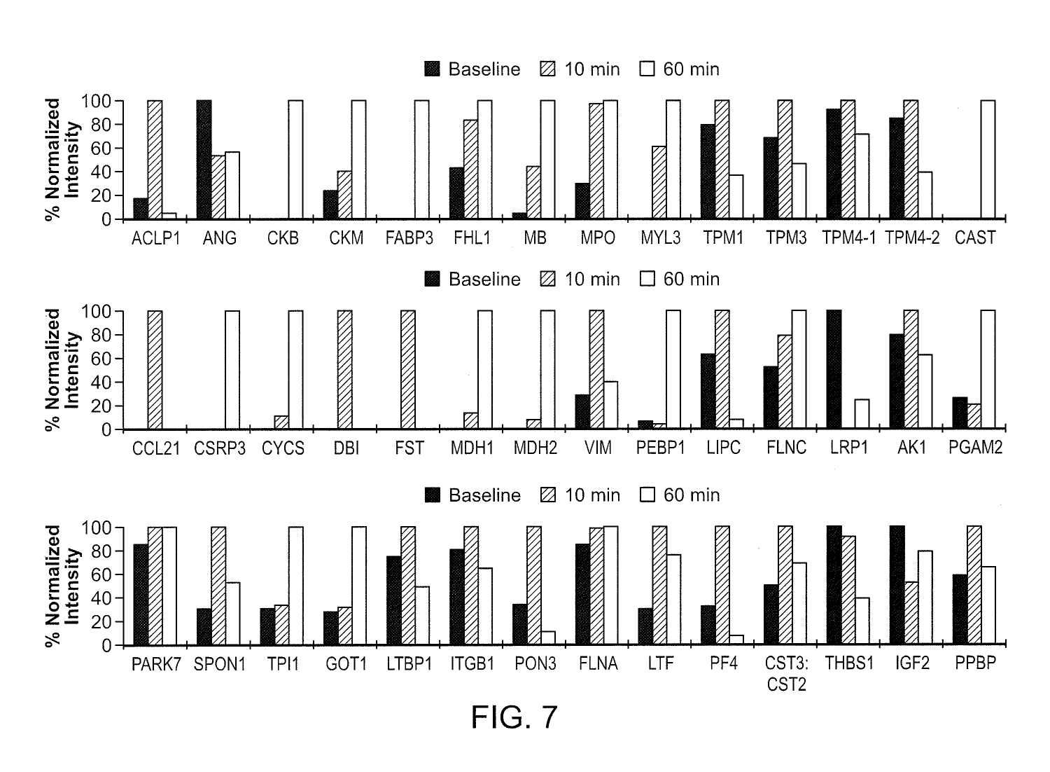

Peptides uniquely derived from 49 of the 82 candidate biomarker proteins (60%) from discovery experiments were detected and sequenced by AIMS in the pool of peripheral plasma from 10 PMI patients. The qualified list contains all of the proteins found in discovery that are known to be associated with myocardial injury, as well as many of the potentially novel biomarkers of CV injury (i.e., those proteins not previously identified in the published literature as being associated with cardiovascular disease, but that were both 5× upregulated and showed clear temporal trends with each patient. For the majority of detected proteins, the relative quantitative information and temporal trends were consistent with that obtained by discovery proteomics of plasma from CS (FIG. 7) though the relative ratios of the MS signals at 10 minutes and/or 60 minutes with respect to baseline were slightly lower in the AIMS data than that observed in the discovery data, possibly due to dilution of the signal in the peripheral blood.

Verification of Candidate Proteins in Peripheral Plasma by Targeted, Quantitative MS Using SID-MRM.

Quantitative verification of candidate biomarkers was conducted using available antibodies as well as by SID-MRM-MS, a targeted, quantitative MS approach (FIG. 1). SID-MRM-MS proved to be essential, as Ab reagents suitable for construction of ELISA assays (i.e., two-per-protein) were available for only 4 of the 42 protein biomarker candidates detected by AIMS). Candidate proteins that were confirmed in the AIMS studies of peripheral blood were then measured in the peripheral plasma of PMI patients using stable isotope dilution (SID) mass spectrometry coupled to multiple reaction monitoring (MRM).

As a demonstration that SID-MRM-MS can be used to assay novel proteins from discovery data in the absence of Abs for quantitative immunoassay construction, SID-MRM-MS strategy (illustrated in FIG. 3) was applied to verify four of the novel, myocardial-enriched proteins, ACLP1, FHL1, MYL3, and tropomyosin 1 (TPM1). Quantitative assays were successfully configured for 15 peptides derived from ACLP1, FHL1, MYL3, and TPM1 using tryptic peptides initially observed in the MS data from the discovery phase (Table 2). Several known markers of myocardial injury were also measured, including C-reactive protein (CRP), myeloperoxidase (MPO), and cardiac troponin T (cTnT), by MRM-MS in the same multiplexed MRM-MS analyses.

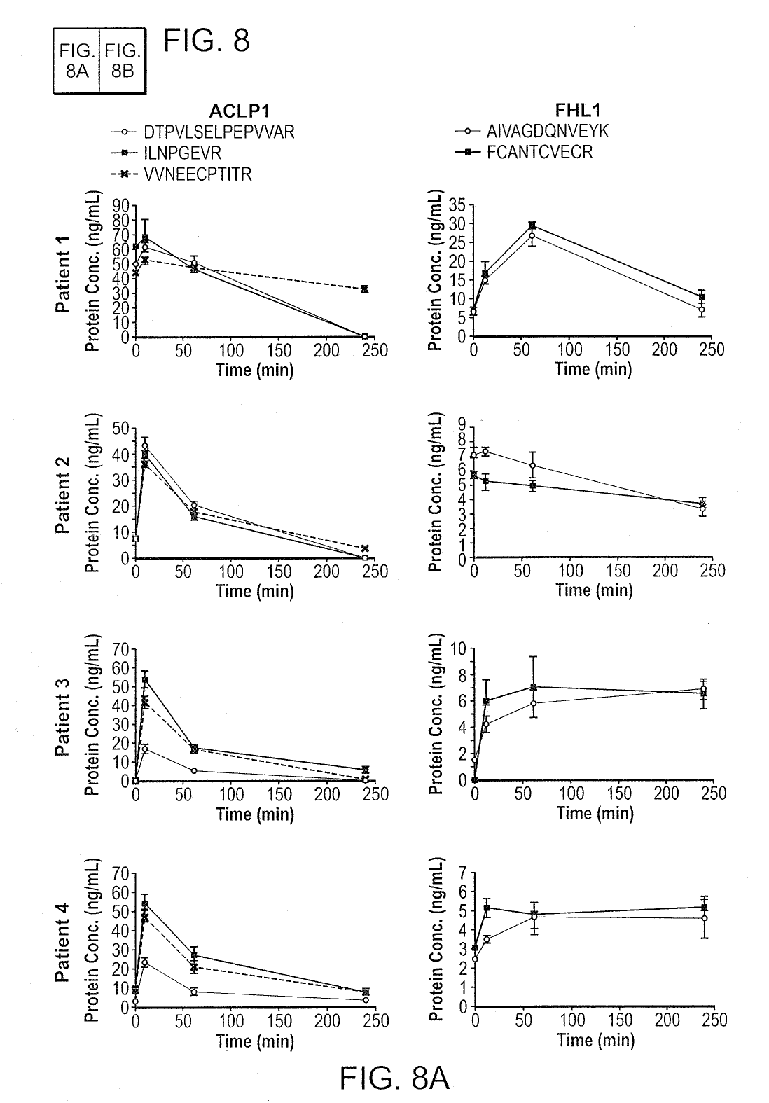

Candidate proteins that were confirmed in the AIMS studies of peripheral plasma from a pool of 10 PMI patients, were measured in the peripheral plasma of 4 individual PMI patients using stable isotope dilution (SID) mass spectrometry coupled to multiple reaction monitoring. All four of the novel protein candidates as well as the three known markers of MI were readily quantified at multiple time points in the patient samples, with measured values ranging from ˜1 ng/mL to 50 ng/mL across all patients and time points (FIG. 8 and Table 5a). For ACLP1, three different signature peptides were readily detected in each patient. The measured concentrations for these peptides were highly consistent with each other, peaking 10 minutes after myocardial injury and then steadily decreasing out to 240 minutes. By 240 min post injury, ACLP1 levels were below detectable limits. For FHL1, MYL3, and TPM1, two signature peptides were detected and quantitatively measured for each protein. In the case of FHL1, measured concentrations peaked at 60 minutes post ablation and then decreased by 240 minutes in 3 out of 4 patients (FIG. 8). Although the measured concentrations obtained for each peptide derived from MYL3 differ by ˜2-fold (most likely due to differing rates of enzymatic digestion; Table 5a), the temporal trends for the pair are consistent across all 4 patients, peaking at 10 minutes and then decreasing in concentration at 240 minutes (FIG. 8). For TPM1, temporal trends show either an increase in measured concentration through 240 minutes post ablation or a leveling in concentration from 60 minutes to 240 minutes in 3 out of 4 patients. Non-uniform behavior of biomarker changes across patients is to be expected due to the variable amount of injury during any given ablation procedure.

Taken together, the MRM-MS results for all four of these novel protein biomarker candidates suggest that they may be early markers of myocardial injury and that additional studies to validate these proteins in larger patient populations are warranted. Of note, the temporal trends for the known MI biomarkers MPO and cTnT were consistent with prior studies (Table 5b). (See Lakkis et al., Circulation 98:1750-55 (1998)) In particular, MRM assays previously configured for C-reactive protein (CRP), myeloperoxidase (MPO), and cardiac troponin T (cTnT) (see Keshishian et al., Mol. Cell. Proteomics 8:1339-2349 (2009)) were used in this study to measure levels of these known markers in the peripheral plasma of 4 individual PMI patients. As expected, MPO levels peaked 10 minutes after injury, whereas cTnT levels were still rising at 240 minutes. For CRP, elevated levels are not observed in these patients at the time points analyzed. This is consistent with previously published data and the literature which indicates that CRP shows elevated levels at 24 hours post myocardial injury (see Keshishian et al., Mol. Cell. Proteomics 8:1339-2349 (2009)). As expected, MPO levels peaked 10 minutes after injury, cTnT levels were still rising at 240 minutes, and CRP levels had not yet begun to rise in these samples.

Verification of Protein Changes by Western Blotting and ELISA.

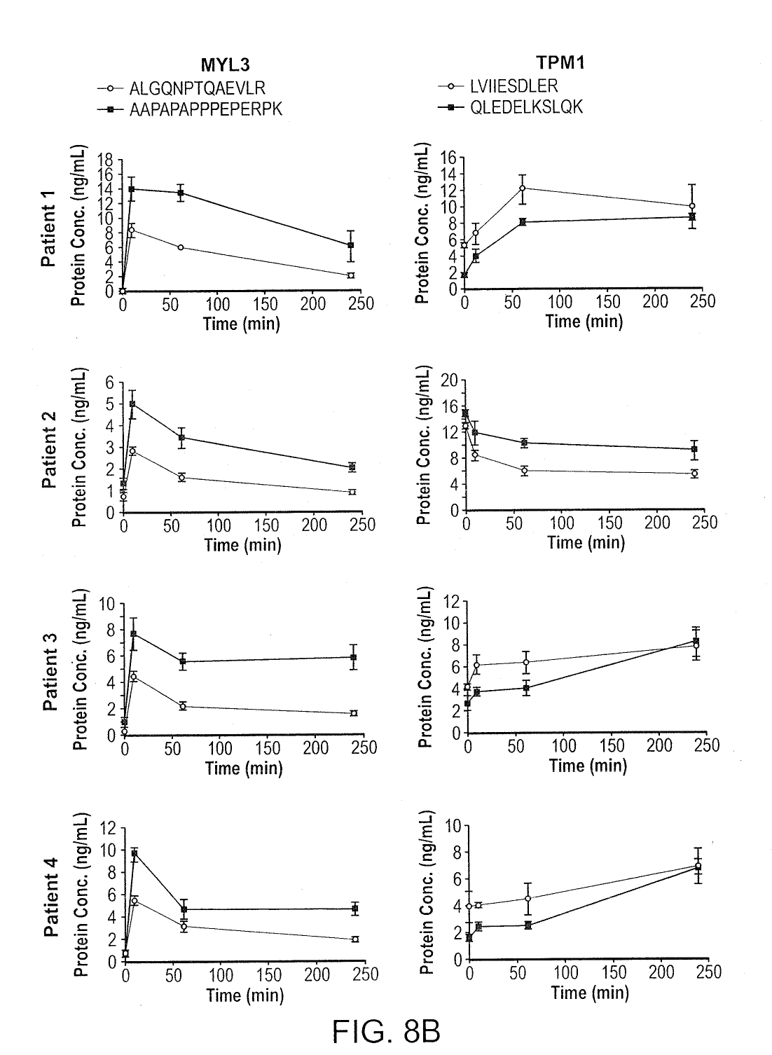

Single antibody reagents were available for 8 of the 82 prioritized candidate biomarkers (Tables 1A and 1B). Reagents for Western blot analyses were used on coronary sinus samples from six additional subjects who underwent the PMI procedure. Only four of the 10 Abs gave useful results by Western. Western blot analyses of midkine (MDK), pleiotrophin (PTN), malate dehydrogenase 1 (MDH1), and ACLP1 were highly consistent with the discovery MS data (FIG. 9a). By contrast, the Abs for MYL3, FHL1, TPM1, and Ryanodine receptor 2, failed to detect endogenous protein in the PMI samples. Several of these Abs (i.e., MYL3, FHL1, TPM1) were able to detect recombinant protein at 10 ng/ml in buffer, but failed to detect these proteins when spiked into human plasma, suggesting interference by other proteins in the plasma matrix.

For angiogenin and midkine, two different Abs were commercially available that recognized distinct regions of each of these proteins, enabling construction of ELISA assays. In addition, for C-C motif chemokine 21 (CCL21) and acyl CoA binding protein (ACBP), ELISA kits were commercially available. ELISA assays for these four proteins were constructed and used for initial candidate verification and to conduct more extensive kinetic analyses using peripheral blood samples from an additional 22 subjects undergoing the ablation procedure (FIG. 9b, left). These studies confirmed highly significant changes in these protein biomarkers as early as 10 minutes after the onset of myocardial injury, with continued elevation of the proteins 2-4 hours after injury.

Further Clinical Validation of Potential Biomarkers.

Using available immunoassays, the specificity of the findings observed in the Planned MI cohort were explored by examining blood samples from patients undergoing routine cardiac catheterization, without the induction of myocardial infarction that occurs in the unique ablation injury model. As seen in FIG. 9b (right panel, control), levels of ACBP, angiogenin, and CCL21 were unchanged up to 60 minutes following routine catheterization in patients presenting with non-acute coronary artery disease and were similar to pre-injury levels of PMI subjects (FIG. 9b, left panel).

Next, it was examined whether these findings were applicable to a cohort of patients with spontaneous MI (SMI) presenting for acute coronary angiography and intervention. The onset of SMIs relative to sample collection was heterogeneous (162±102 minutes), as was the extent of myocardial injury. The baseline characteristics for these patients are listed in Table 3. As seen in FIG. 9b right, significantly higher levels of these three proteins were observed in the SMI patients, as compared to levels in patients who presented to the cardiac catheterization suite with non-acute coronary artery disease (control). SMI levels were similar to peak levels seen in PMI. Of note, cardiac catherization alone was associated with changes in the levels of other proteins, midkine, pleiotrophin, decorin, and secreted frizzle related protein levels as observed with in-house constructed ELISA assays. Thus, proteins with changes that were not specific to myocardial injury and that may instead reflect procedural events such as arteriotomy, catheter manipulation, or drug therapy were eliminated for further evaluation using the appropriate patient controls.

Finally, since proteins were released early after the onset of the planned myocardial infarction, we next examined whether levels were also increased in the setting of reversible myocardial ischemia. A total of 52 patients undergoing exercise stress testing with myocardial perfusion imaging served as the study population: 26 with no evidence of ischemia (controls) and 26 patients with evidence of inducible ischemia (cases). The baseline characteristics and stress test performance parameters for these patients are listed in Table 6. The mean ages of the two groups were comparable, though as expected, patients with inducible ischemia had slightly more cardiac risk factors (3.0±0.9 vs. 2.1±0.9) and were more likely to have a documented history of coronary disease.

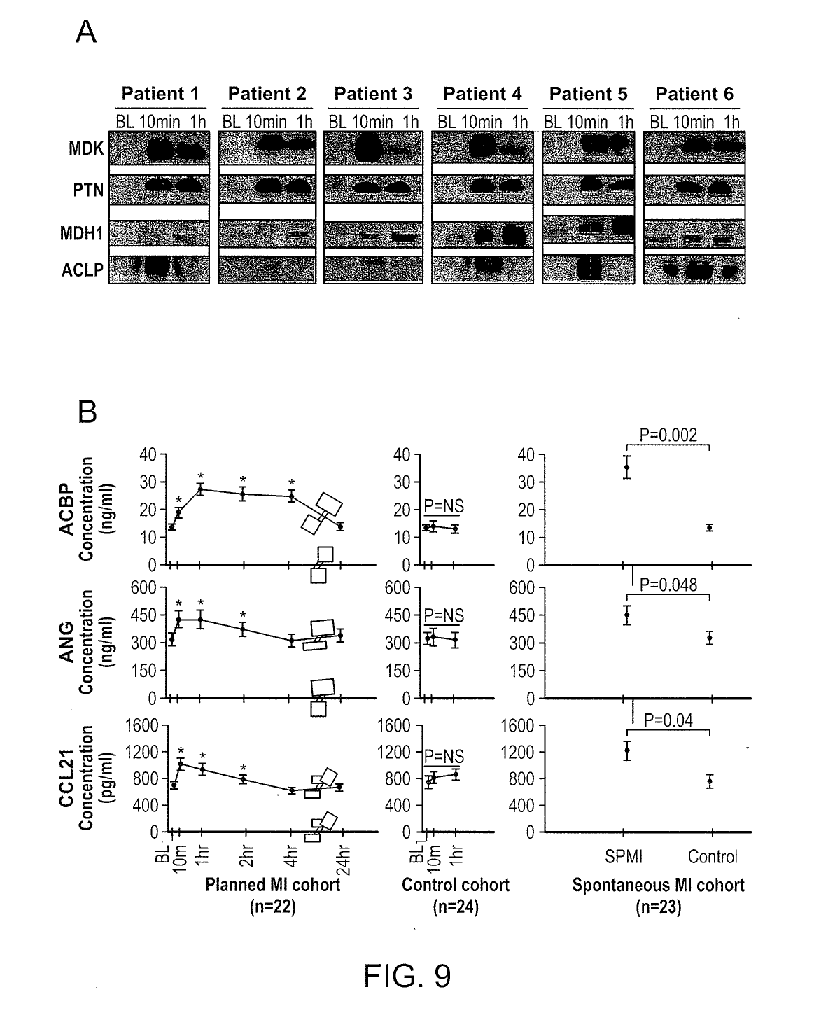

The exercise stress test results of cases and controls are shown in FIG. 10. By design, all 26 cases had reversible perfusion defects, with the mean percentage of myocardium with a reversible perfusion defect being 17±8%, whereas, no controls had any degree of a reversible perfusion defect. Of note, it was interesting to find that for two of the proteins, ACBP and ANG, baseline levels were higher in the ischemic as compared to the at-risk control patients. Furthermore, for ACBP, a modest augmentation in protein levels in the setting of myocardial ischemia was also documented that was not observed in the control subjects.

### Discussion

Although emerging proteomics profiling technologies hold enormous promise for illuminating new biomarkers, successful applications to human disease are still lacking. This is due, in large part, to the lack of a coherent pipeline enabling systematic building of credentialing information around biomarker candidate proteins emerging from discovery proteomics experiments. It has previously been posited that a testable discovery-through-verification biomarker pipeline that includes, first unbiased discovery in proximal fluid or tissue; second, qualification of discovered candidates in peripheral blood of additional patient samples; and third, verification of qualified, discovered candidates in peripheral blood using targeted, quantitative MS-based assays, specifically MRM-MS and SISCAPA. (See Rifal et al., Nat. Biotechnol. 24:971-83 (2006)) Here the initial application of that biomarker pipeline was demonstrated. (FIG. 1). The discovery, qualification and verification steps systematically informed the next stage of the pipeline and the analyses took specific advantage of key attributes of the MS-based technology platforms used at each stage.

The pipeline approach was applied, beginning with discovery, to a unique clinical model of MI that allowed for precise kinetic analysis in patients who serve as their own biological controls. Coronary sinus catheterization provided the opportunity to sample directly from the organ of interest. This approach enabled the use of a proximal fluid of the heart for discovery of candidate biomarker proteins rather than peripheral plasma where proteins arising from the myocardium would have been diluted. The consistent temporal changes of candidate biomarkers within and across patients (FIG. 7) underscores the biological plausibility of the observed association between proteomic changes and MI. This study emphasizes the important point that small numbers of samples may be employed for discovery if the effect size is large. The current study began with samples from three time points and in three patients undergoing PMI, focused on changes of at least five-fold in protein abundance before identifying a protein as a candidate. This experimental design enhanced the power to identify statistically meaningful changes.

Using untargeted, data dependent LC-MS/MS based proteomics for discovery, 1086 unique total proteins with two or more peptides and a FDR of ≦1% in the plasma from the coronary sinus were identified, or 992 proteins after excluding immunoglobulins and common contaminants such as keratins. The identified proteins spanned ca. 6-7 orders of magnitude of abundance, based on detection of peptides from REG3, IGFBP4 and ICN2 that are known to be present at 1-130 nanogram/mL levels in normal patient plasma. (See Whiteaker et al., Anal Biochem 362:44-54 (2007)). Consistent with prior studies (see States et al., Nat. Biotechnol. 24:333-38 (2006); Schenk et al., BMC Med. Genomics 1:41-68 (2008)), the pipeline described herein underscores the need for abundant protein depletion combined with extensive peptide- or protein-level fractionation prior to LC-MS/MS for identification of proteins present at low ng/mL range in plasma. In the present study, discovery samples of three time points from three patients yielded over 700 sample sub-fractions, necessitating approximately 2800 hours of instrument time on the Orbitrap for LC-MS/MS analyses. The resulting list of proteins detected with very high confidence in plasma adds to the list of high quality studies of the human plasma proteome. (See States et al., Nat. Biotechnol. 24:333-38 (2006); Schenk et al., BMC Med. Genomics 1:41-68 (2008) and references cited therein)

Qualification is an essential element of the pipeline for biomarker prioritization (FIG. 1), since considerable resources are necessary to develop either SID-MRM-MS or ELISA-based assays. AIMS serves as the ideal next step following the acquisition of discovery proteomics data. AIMS takes advantage of the low parts per million (ppm) mass accuracy and high (≧60,000) resolution for peptide precursor masses, together with fast and sensitive sequencing of peptides that is possible with modern hybrid mass spectrometers such as the Orbitrap mass spectrometer. In contrast to discovery experiments in which proteins are identified based upon a stochastic sampling of the peptide precursor masses, AIMS is a targeted MS approach in which MS/MS spectra are triggered and acquired only when an accurate mass and charge pair on the inclusion list are detected. Prior studies have documented that any protein detected by AIMS in plasma can be quantified by MRM-MS. (See Jaffe et al., Mol. Cell. Proteomics 7:1952-62 (2008)) Therefore, AIMS is well suited as a bridge between discovery and targeted, quantitative MS-based assay development, enabling large numbers of candidates to be qualified (typically ca. 100 proteins/LC-MS/MS run). AIMS is a particularly useful bridging tool for the proteins that are completely novel. Here the initial qualification of 60% of the proteins on the inclusion list was demonstrated, thus prioritizing them for more resource-intensive SID-MRM-MS and Ab reagent development. It is important to note that the AIMS method is not a filter. Proteins not detected by AIMS remain on the list for assay development, but are flagged as likely requiring more extensive fractionation or use of anti-protein or anti-peptide immunoaffinity enrichment in order to construct a useful assay. (See Anderson et al., J. Proteome Res 3:235-44 (2004); Kuhn et al., Clin. Chem. 55:1108-17 (2009); Whiteaker et al., Anal. Biochem. 362:44-54 (2007); Hoofnagle et al., Clin. Chem. 54:1796-1804 (2008)) In addition, proteins containing modifications such as phosphorylation or sequence isoforms or mutations can also be targeted by AIMS, thereby providing a rapid way to test for the presence of proteins containing these modifications in any matrix (tissue, cells or biofluids).

The third step of the pipeline is verification (see Rifai et al., Nat. Biotechnol. 24:971-83 (2006)) using SID-MRM-MS or ELISA for the minority of cases where Abs are available (FIG. 1). Abs suitable for construction of ELISA assays were available for only four of the novel candidate biomarker proteins that emerged from discovery. Single Ab reagents and commercial ELISA assays were available for 10 more proteins, although the credentialing of these antibodies was highly variable. In the initial verification studies, Western blotting failed to document changes noted by mass spectrometry in three cases. Ongoing studies are presently examining the cause of the discrepancies between the MS and Western findings. In principal, antibody (Ab)-based measurements could be used at all steps in the validation process. However, few immunoassay-grade antibodies of sufficient quality and number (2-per protein candidate) are available, and developing a new, clinically deployable immunoassay is expensive and time consuming, which restricts such development to a short list of already highly credentialed candidates. (See Rifai et al., Nat. Biotechnol. 24:971-83 (2006)).

Consequently, quantitative SID-MRM-MS assays were developed for four of the novel, heart-specific proteins discovered in this study, together with additional cardiovascular-related proteins already in clinical use or of growing interest. (See Keshishian et al., Mol. Cell. Proteomics 8:1339-2349 (2009)) Highly consistent temporal trends were observed for two or three peptides measured for each of the novel candidate proteins across 4 patients. Additionally, there was a high degree of correlation between AIMS and SID-MRM results for the novel candidates. All four proteins were found to be elevated in abundance at 10 and/or 60 minutes with respect to baseline by AIMS using pooled patient plasma and SID-MRM using individual patient plasma. However levels of MYL3 decreased from 10 min to 60 min sample in all 4 patients as measured by SID-MRM while the levels increased slightly in the AIMS experiment. This is possibly due to dilution of these proteins in the plasma pool used for AIMS whereas individual patient plasma was processed and analyzed for SID-MRM-MS.

The need for alternate methods to rapidly configure quantitative assays to credential novel protein biomarkers is highlighted by a recent study of pancreatic cancer. (See Faca et al., PLoS Med. 5:e123 (2008)) Over 600 proteins were quantified in plasma of which 165 (ca. 27%) were found to change in abundance. In their verification studies, Ab reagents for only ca. 11 of these proteins were available, including CA-19-9, a marker of pancreatic cancer in clinical use. Due to the lack of Ab reagents, no follow-up studies were performed for the remaining proteins of interest.

With regards to the biological findings, the unbiased analysis described herein “rediscovered” many of the known cardiovascular biomarkers, including creatine kinase, myoglobin, fatty acid binding protein and myeloperoxidase. The new data also extend prior work by identifying many new proteins not previously associated with acute myocardial injury in humans. Angiogenin, is a potent endothelial growth factor. While the mechanism of angiogenin generation remain incompletely understood, one study has demonstrated that angiogenin gene transfer induces angiogenesis and modifies left ventricular remodeling in rats with myocardial infarction. (See Zhao et al., J Mol Med 84:1033-46 (2006)) More recently, one other group has identified elevated angiogenin levels in subjects presenting with acute coronary syndromes and higher angiogenin levels were associated with adverse events following admission with ACS. (See Tello-Montoliu et al., Eur. Heart J. 28:3006-11 (2007)) The documentation of elevated angiogenin levels in subjects with coronary artery disease without any evidence of unstable symptoms thus extends these prior observations. Rapid rises in levels of CCL21, a known T cell chemokine, were also observed, though data suggest that this protein may be highly expressed in the heart as well (http://www.genecards.org/cgi-bin/carddisp.pl?gene=CCL21). Finally, ACLP is a secreted factor most highly expressed in the vasculature (see Layne et al., Mol. Cell. Biol. 21:5256-61 (2001); Layne et al., Circ. Res. 90:728-36 (2002)) and ACLP knockout mice have a severe wound healing defect. (See Layne et al., Circ. Res. 90:728-36 (2002)) The inferred relationships with MI based on prior studies merit rigorous examination in relevant animal models.

The approach described herein to enhance biomarker and pathway discovery emphasized the in-depth analysis of a small, extremely well-phenotyped patient cohort. Promising proteins were then validated in additional more heterogeneous cohorts. However, the present study has several limitations that should be considered. First, although serial sampling in patients serving as their own biological controls helped diminish inter-individual variability and signal-to-noise issues, the discovery study population was nevertheless very small. Thus, it is important to note that changes in proteins that failed to reach nominal significance in the present study still may be scientifically important and bear further investigation. Second, a human clinical scenario characterized by a marked cardiac perturbation was selected. This may have influenced which proteins were altered, the magnitude of the perturbations, and the ultimate clinical utility of the candidate markers, although the finding that several of the biomarkers appear elevated in subjects with spontaneous MI and reversible myocardial ischemia, suggests that the that model has clinical relevance. Finally, although the proteomics markers identified herein had excellent discriminatory power in subjects with spontaneous ischemic disease and myocardial injury, these findings must be further evaluated in larger populations, which will also permit comparison to and adjustment for traditional cardiovascular risk factors and other clinical parameters. Further testing of putative markers in larger cohorts will provide the opportunity for exploration of subgroups of interest including those based on gender, race, and co-morbidities, which we were underpowered to do.

In summary, the present study has established a biomarker pipeline to identify many potential early markers of myocardial injury. It has been demonstrated that this pipeline can be successfully applied to credential candidate biomarkers MS-based targeted assays and immunoassays when reagents exist. These methods can be applied to interrogate the remaining candidates from the discovery proteomics studies having first focused resources on cardiac-enriched targets of potential biological interest. The list includes several proteins that may indeed serve as markers of reversible myocardial ischemia, for which no circulating biomarkers presently exist. The biomarker discovery pipeline demonstrated here will allow one skilled in the art to “overlay” new biomarkers onto established markers to create multimarker risk scores. It is anticipated that some new markers will be uncorrelated or “orthogonal” to existing markers, thus providing additional information for cardiovascular disease management.

TABLE 2
Target proteins and their signature peptides for MRM-MS assay development.

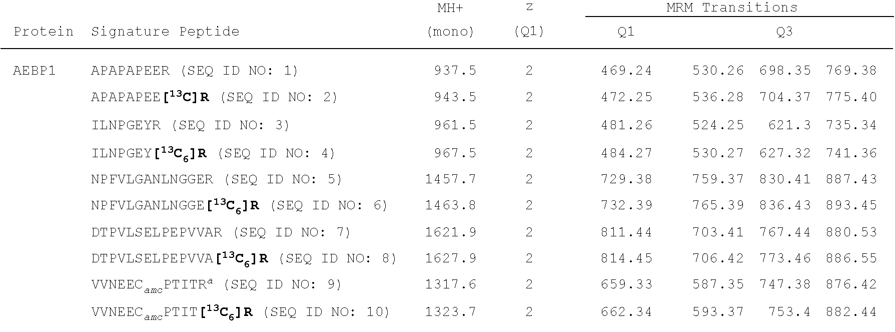

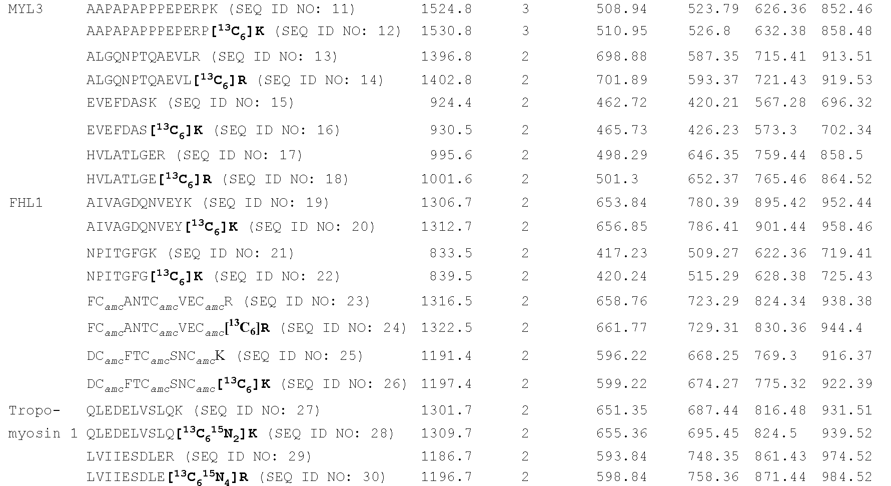

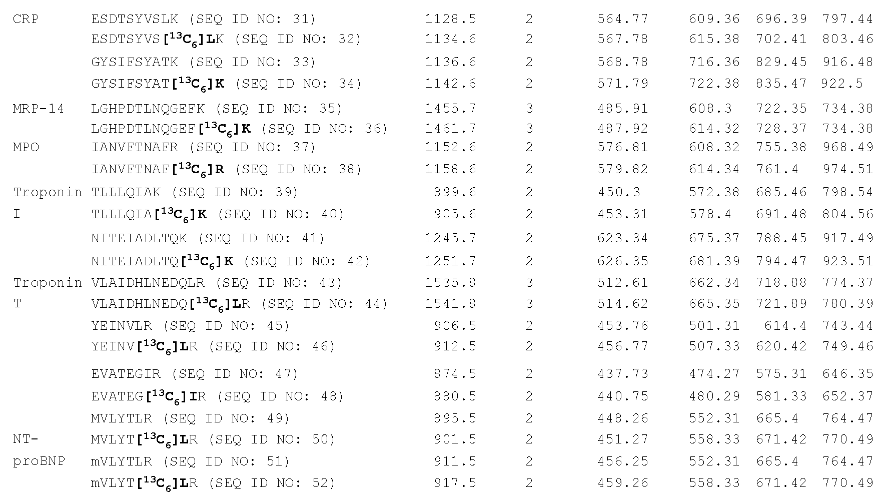

Unlabeled and corresponding [¹³C], and [¹³C¹⁵N] labeled peptides were synthesized for optimization and employment of stable isotope dilution, multiple reaction monitoring mass spectrometry (SID-MRM-MS). Uniformly labeled amino acids are indicated in bold. Came = carbamidomethyl cysteines

TABLE 3
Baseline clinical characteristics of study subjects.

| | Planned MI | Planned MI | Spontaneous MI | |
|---|---|---|---|---|
| | Cohort | Cohort | Cohort | Control |
| | (Discovery) | (Validation) | (Validation) | Cohort |
| | (n = 3) | (n = 22 | (n = 23) | (n = 24) |
| Age, years | 64 ± 16 | 61.1 ± 12.4 | 59.3 ± 12.8 | 57.2 ± 11.1 |
| Male sex (%) | 33 | 47.1 | 73.9 | 57.9 |
| Caucasian Race, (%) | 100 | 76.5 | 87 | 94.7 |
| Creatine baseline | 0.86 ± 0.15 | 1.0 ± 0.2 | 1.4 ± 0.8 | 1.1 ± 0.3 |
| Peak troponin T (ng/mL) | 7.8 ± 5.3 | 4.0 ± 2.9 | 6.3 ± 6.2 | <0.01* |
| Peak creatine kinase (U/L) | 1301 ± 521 | 1064 ± 375 | 1592 ± 1335 | 81 ± 35* |
| Peak creatine kinase-MB (ng/mL) | 194 ± 58 | 150 ± 64 | 220 ± 294 | 2.4 ± 1.2* |
| Total cholesterol | N/A | 159 ± 34 | N/A | 164 ± 36 |

TABLE 4
Summary of 82 protein biomarker candidates detected in coronary sinus plasma of PMI patients by discovery
proteomics and the 42 proteins that were qualified as detectable in peripheral plasma of PMI patients.

| | | | | | | | | Proteins Not Detected in | |
|---|---|---|---|---|---|---|---|---|---|
| Proteins Detected in Peripheral Plasma by AIMS | | | | | | | | Peripheral Plasma by AIMS | |
| | Candidate Biomarker | AIMS, Total Intensity | | | Ratio | | | | Candidate Biomarker |
| # | Protein | Baseline | 10 min | 60 min | 10:BL | 60:BL | 60:10 | # | Protein |
| 1 | ACLP Aortic | 199 | 1130 | 64 | 5.7 | 0.3 | 0.1 | 43 | MDK Midkine |
| | carboxypeptidase-like | | | | | | | | |
| | protein 1 | | | | | | | | |
| 2 | ANG Angiogenin | 10800 | 5880 | 6110 | 0.5 | 0.6 | 1.0 | 44 | MYBPC1 myosin |
| | | | | | | | | | binding protein C, |
| | | | | | | | | | slow type isoform 1 |
| 3 | CKB Creatine kinase B- | 0 | 0 | 849 | 0.0 | >20 | >20 | 45 | SFRP1 Secreted |
| | type | | | | | | | | frizzled-related |
| | | | | | | | | | protein 1 |
| 4 | CKM Creatine kinase M- | 7270 | 12300 | 30700 | 1.7 | 4.2 | 2.5 | 46 | TPM2 |
| | type | | | | | | | | Tropomyosin 2 |
| 5 | FABP3 Fatty acid-binding | 0 | 0 | 1920 | 0.0 | >20 | >20 | 47 | ALMS1 ALMS1 |
| | protein, heart | | | | | | | | |
| 6 | FHL1 Four and a half LIM | 322 | 619 | 740 | 1.9 | 2.3 | 1.2 | 48 | ALPK2 heart |
| | domains 1 | | | | | | | | alpha-kinase |
| 7 | MB Myoglobin | 1920 | 15500 | 34800 | 8.1 | 18.1 | 2.2 | 49 | ANKRD26 |
| | | | | | | | | | Isoform 2 of |
| | | | | | | | | | Ankyrin repeat |
| | | | | | | | | | domain-containing |
| | | | | | | | | | protein 26 |
| 8 | MPO Isoform H7 of | 5360 | 17600 | 18100 | 3.3 | 3.4 | 1.0 | 50 | BMP1 Isoform |
| | Myeloperoxidase | | | | | | | | BMP1-3 of Bone |
| | | | | | | | | | morphogenetic |
| | | | | | | | | | protein 1 |
| 9 | MYL3 Myosin light chain 3 | 0 | 702 | 1140 | >20 | >20 | 1.6 | 51 | CSRP1 Cysteine |
| | | | | | | | | | and glycine-rich |
| | | | | | | | | | protein 1 |
| 10 | TPM1 Isoform 4 of | 2820 | 3530 | 1290 | 1.3 | 0.5 | 0.4 | 52 | CTTNBP2 |
| | Tropomyosin alpha | | | | | | | | Cortactin-binding |
| | | | | | | | | | protein 2 |
| 11 | TPM3 tropomyosin 3 | 2400 | 3500 | 1620 | 1.5 | 0.7 | 0.5 | 53 | DCN Isoform A of |
| | isoform 1 | | | | | | | | Decorin |
| 12 | TPM4 Isoform 1 of | 6970 | 7530 | 5370 | 1.1 | 0.8 | 0.7 | 54 | DNAH17 Isoform |
| | Tropomyosin alpha | | | | | | | | 1 of Dynein heavy |
| | | | | | | | | | chain 17, axonemal |
| 13 | TPM4 Isoform 2 of | 3060 | 3570 | 1390 | 1.2 | 0.5 | 0.4 | 55 | DPYSL3 DPYSL3 |
| | Tropomyosin alpha | | | | | | | | protein |
| 14 | CAST calpastatin isoform a | 0 | 0 | 95 | 0.0 | >20 | >20 | 56 | FAT2 |
| | | | | | | | | | Protocadherin Fat 2 |
| 15 | CCL21 C-C motif | 0 | 116 | 0 | >20 | 0.0 | 0.0 | 57 | FRAS1 Isoform 1 of |
| | chemokine 21 | | | | | | | | Extracellular matrix |
| | | | | | | | | | protein FRAS1 |
| 16 | CSRP3 Cysteine and | 0 | 0 | 169 | 0.0 | >20 | >20 | 58 | HERC2 Probable E3 |
| | glycine-rich protein 3 | | | | | | | | ubiquitin-protein |
| 17 | CYCS Cytochrome c | 0 | 112 | 988 | >20 | >20 | 8.8 | 59 | HERC2P2 Similar |
| | | | | | | | | | to Hect domain |
| | | | | | | | | | and RLD 2 |
| 18 | DBI Isoform 2 of Acyl-CoA- | 0 | 4 | 0 | >20 | 0.0 | 0.0 | 60 | HIVEP2 |
| | binding protein | | | | | | | | Transcription |
| | | | | | | | | | factor HIVEP2 |
| 19 | FST Isoform 1 of Follistatin | 0 | 379 | 0 | >20 | 0.0 | 0.0 | 61 | HRNR Hornerin |
| 20 | MDH1 Malate | 0 | 644 | 4930 | >20 | >20 | 7.7 | 62 | IMMT Isoform 1 of |
| | dehydrogenase, | | | | | | | | Mitochondrial inner |
| | cytoplasmic | | | | | | | | membrane protein |
| 21 | MDH2 Malate | 0 | 122 | 1750 | >20 | >20 | 14.3 | 63 | KIAA0515 |
| | dehydrogenase, | | | | | | | | hypothetical |
| | mitochondrial | | | | | | | | protein LOC84 |
| 22 | VIM Vimentin | 159 | 568 | 221 | 3.6 | 1.4 | 0.4 | 64 | LRP6 Low-density |
| | | | | | | | | | lipoprotein |
| | | | | | | | | | receptor-related |
| | | | | | | | | | protein 6 |
| 23 | PEBP1 | 346 | 204 | 6390 | 0.6 | 18.5 | 31.3 | 65 | MYH13 Myosin-13 |
| | Phosphatidylethanolamine- | | | | | | | | |
| | binding protein 1 | | | | | | | | |
| 24 | LIPC Hepatic | 349 | 562 | 41 | 1.6 | 0.1 | 0.1 | 66 | NEB Nebulin |
| | triacylglycerol lipase | | | | | | | | |
| 25 | FLNC Isoform 1 of Filamin-C | 515 | 776 | 1000 | 1.5 | 1.9 | 1.3 | 67 | NOPE Isoform 1 of |
| | | | | | | | | | Neighbor of punc e11 |
| 26 | LRP1 14 kDa protein | 682 | 0 | 162 | 0.0 | 0.2 | >20 | 68 | PAPPA |
| | | | | | | | | | Pappalysin-1 |
| 27 | AK1 Adenylate kinase 1 | 738 | 940 | 584 | 1.3 | 0.8 | 0.6 | 69 | PF4V1 Platelet |
| | | | | | | | | | factor 4 variant |
| 28 | PGAM2 Phosphoglycerate | 885 | 681 | 3380 | 0.8 | 3.8 | 5.0 | 70 | PKHD1 Isoform 1 |
| | mutase 2 | | | | | | | | of Fibrocystin |
| 29 | PARK7 Protein DJ-1 | 1040 | 1220 | 1210 | 1.2 | 1.2 | 1.0 | 71 | PLXDC2 Isoform |
| | | | | | | | | | 1 of Plexin |
| | | | | | | | | | domain-containing |
| | | | | | | | | | protein 2 |
| 30 | SPON1 Spondin-1 | 1350 | 4490 | 2360 | 3.3 | 1.7 | 0.5 | 72 | PTN Pleiotrophin¹ |
| 31 | TPI1 Isoform 1 of | 1490 | 1630 | 4880 | 1.1 | 3.3 | 3.0 | 73 | RSF1 remodeling |
| | Triosephosphate | | | | | | | | and spacing factor |
| | isomerase 1 | | | | | | | | |
| 32 | GOT1 Aspartate | 1700 | 1970 | 6280 | 1.2 | 3.7 | 3.2 | 74 | RYR2 Isoform 1 |
| | aminotransferase, | | | | | | | | of Ryanodine |
| | cytoplasmic | | | | | | | | receptor 2 |
| 33 | LTBP1 latent transforming | 1820 | 2450 | 1190 | 1.3 | 0.7 | 0.5 | 75 | SACS Isoform 1 |
| | growth factor beta bind. | | | | | | | | of Sacsin |
| | protein 1 | | | | | | | | |
| 34 | ITGB1 integrin beta 1 | 2680 | 3360 | 2170 | 1.3 | 0.8 | 0.6 | 76 | SFTPD Pulmonary |
| | isoform 1A protein | | | | | | | | surfactant-associated |
| | | | | | | | | | protein D |
| 35 | PON3 Serum | 3570 | 10700 | 1070 | 3.0 | 0.3 | 0.1 | 77 | SMG1 Isoform 1 of |
| | paraoxonase/lactonase 3 | | | | | | | | Serine/threonine- |
| | | | | | | | | | protein kinase SMG1 |
| 36 | FLNA filamin A, alpha | 5760 | 6710 | 6850 | 1.2 | 1.2 | 1.0 | 78 | TAGLN |
| | isoform 1 | | | | | | | | Transgelin |
| 37 | LTF Growth-inhibiting | 7500 | 26400 | 19900 | 3.5 | 2.7 | 0.8 | 79 | THBS3 |
| | protein 12 | | | | | | | | Thrombospondin-3 |
| 38 | PF4 Platelet factor 4 | 13500 | 43900 | 2640 | 3.3 | 0.2 | 0.1 | 80 | TIAM1 T-lymphoma |
| | | | | | | | | | invasion and |
| | | | | | | | | | metastasis- |
| | | | | | | | | | inducing protein 1 |
| 39 | CST3; CST2 Cystatin-C | 29200 | 60000 | 40400 | 2.1 | 1.4 | 0.7 | 81 | TNNT2 Isoform 1 |
| | | | | | | | | | of Troponin T, |
| | | | | | | | | | cardiac muscle |
| 40 | THBS1 Thrombospondin-1 | 29600 | 26900 | 11100 | 0.9 | 0.4 | 0.4 | 82 | TPR nuclear pore |
| | | | | | | | | | complex-associated |
| | | | | | | | | | protein TPR |
| 41 | IGF2 insulin-like growth | 47500 | 24000 | 37100 | 0.5 | 0.8 | 1.5 | | |
| | factor 2 isoform 2 | | | | | | | | |
| 42 | PPBP Platelet basic | 66700 | 117000 | 74400 | 1.8 | 1.1 | 0.6 | | |
| | protein | | | | | | | | |

TABLE 5A
Summary of MRM results for four novel biomarker candidates (Inter-assay % CV is calculated based
on the average of all 3 process replicates for each time point. n/d = no detection of analyte)

| | | AEBP 1 | | | | | | FHL 1 | | | |
|---|---|---|---|---|---|---|---|---|---|---|---|
| | | DTPVLSELPEPVVAR | | VVNEECPTITR | | ILNPGEYR | | AIVAGDQNVEYK | | FCANTCVECR | |
| | | (SEQ ID NO: 7) | | (SEQ ID NO: 53) | | (SEQ ID NO: 3) | | (SEQ ID NO: 19) | | (SEQ ID NO: 54) | |
| | | Avg. | Inter- | Avg. | Inter- | Avg. | Inter- | Avg. | Inter- | Avg. | Inter- |
| | | Conc. | assay | Conc. | assay | Conc. | assay | Conc. | assay | Conc. | assay |
| | | (ng/mL) | % CV | (ng/mL) | % CV | (ng/mL) | % CV | (ng/mL) | % CV | (ng/mL) | % CV |
| Patient 1 | Baseline | 44.83 | 16.1 | 40.78 | 24.6 | 62.41 | 2.2 | 8.16 | 2.6 | 7.54 | 28.7 |
| | 10 min | 63.11 | 26.6 | 55.37 | 16.2 | 69.46 | 26.7 | 15.66 | 19.0 | 17.62 | 27.0 |
| | 60 min | 51.95 | 15.2 | 48.75 | 13.9 | 48.99 | 10.5 | 28.32 | 18.3 | 30.72 | 5.2 |
| | 240 min | n/d | — | 33.89 | 12.3 | n/d | — | 7.46 | 44.5 | 10.86 | 27.9 |
| Patient 2 | Baseline | 9.86 | 30.9 | 9.12 | 79.0 | 8.12 | 65.4 | 7.17 | — | 5.72 | 52.4 |
| | 10 min | 44.02 | 11.3 | 37.06 | 9.6 | 40.61 | 14.2 | 7.38 | 7.6 | 5.28 | 19.0 |
| | 60 min | 19.47 | 18.0 | 18.23 | 21.8 | 16.25 | 20.3 | 6.45 | 24.0 | 4.97 | 13.0 |
| | 240 min | n/d | — | 3.64 | 14.8 | n/d | — | n/d | — | 3.69 | 19.9 |
| Patient 3 | Baseline | n/d | — | n/d | — | n/d | — | 1.63 | 21.8 | n/d | — |
| | 10 min | 17.43 | 25.2 | 42.41 | 13.1 | 54.42 | 15.3 | 4.22 | 26.8 | 6.02 | 48.3 |
| | 60 min | 5.15 | 19.7 | 16.75 | 24.0 | 17.38 | 19.8 | 5.80 | 31.1 | 7.12 | 56.2 |
| | 240 min | n/d | — | n/d | — | 5.26 | 23.2 | 6.82 | 18.0 | 6.57 | 27.4 |
| Patient 4 | Baseline | 2.47 | 18.5 | 9.58 | 23.2 | 8.61 | 5.1 | 2.45 | 24.3 | 3.05 | 9.02 |
| | 10 min | 22.96 | 20.9 | 47.71 | 10.5 | 54.12 | 16.1 | 3.47 | 5.1 | 5.19 | 16.71 |
| | 60 min | 7.69 | 30.3 | 20.64 | 30.1 | 26.40 | 31.4 | 4.63 | 32.6 | 4.77 | 25.50 |
| | 240 min | 2.39 | 31.7 | 5.81 | 30.0 | 6.36 | 26.1 | 4.61 | 38.8 | 5.19 | 19.13 |

| | | Myosin Light Chain 3 | | | | Tropomyosin 1 | | | |
|---|---|---|---|---|---|---|---|---|---|
| | | ALGQNPTQAEVLR | | AAPAPAPPPEPERPK | | LVIIESDLER | | QLEDELVSLQK | |
| | | (SEQ ID NO: 13) | | (SEQ ID NO: 11) | | (SEQ ID NO: 29) | | (SEQ ID NO: 27) | |
| | | Avg. | Inter- | Avg. | Inter- | Avg. | Inter- | Avg. | Inter- |
| | | Conc. | assay | Conc. | assay | Conc. | assay | Conc. | assay |
| | | (ng/mL) | % CV | (ng/mL) | % CV | (ng/mL) | % CV | (ng/mL) | % CV |
| Patient 1 | Baseline | n/d^{b} | — | n/d | — | 5.21 | 7.0 | 1.44 | 31.3 |
| | 10 min | 8.35 | 20.9 | 14.06 | 17.4 | 6.57 | 34.6 | 3.79 | 39.4 |
| | 60 min | 6.03 | 8.8 | 13.50 | 15.4 | 12.10 | 26.3 | 8.02 | 10.0 |
| | 240 min | 2.01 | 30.9 | 6.07 | 59.9 | 10.06 | 47.3 | 8.73 | 29.2 |
| Patient 2 | Baseline | 0.72 | 52.0 | 1.30 | 42.4 | 12.89 | 50.7 | 14.85 | 42.1 |
| | 10 min | 2.83 | 12.6 | 4.96 | 24.3 | 8.27 | 17.3 | 11.77 | 27.8 |
| | 60 min | 1.57 | 19.0 | 3.40 | 24.1 | 5.92 | 19.1 | 10.15 | 13.5 |
| | 240 min | 0.85 | 21.4 | 2.00 | 20.0 | 5.61 | 19.5 | 9.23 | 27.7 |
| Patient 3 | Baseline | 0.35 | 2.2 | 0.82 | 4.7 | 4.01 | 3.8 | 2.39 | 41.5 |
| | 10 min | 4.36 | 16.0 | 7.62 | 28.7 | 6.11 | 25.8 | 3.49 | 22.0 |
| | 60 min | 2.09 | 24.4 | 5.52 | 20.8 | 6.29 | 29.2 | 3.79 | 33.6 |
| | 240 min | 1.46 | 29.2 | 5.78 | 28.8 | 7.95 | 32.5 | 8.28 | 29.5 |
| Patient 4 | Baseline | 0.56 | 7.9 | 0.95 | 19.0 | 3.87 | 43.5 | 1.49 | 23.5 |
| | 10 min | 5.46 | 13.8 | 9.59 | 11.9 | 4.01 | 1.6 | 2.43 | 26.4 |
| | 60 min | 3.14 | 29.3 | 4.63 | 27.4 | 4.45 | 45.9 | 2.42 | 14.3 |
| | 240 min | 1.84 | 14.7 | 4.55 | 22.6 | 6.96 | 34.7 | 6.89 | 13.9 |

TABLE 5B
Summary of MRM results for known makers of cardiovascular injury (Inter-
assay % CV is calculated based on the average of all 3 process replicates
for each time point. n/d = no detection of analyte)

| | | C reactive protein | | | | MPO | | Troponin T | |
|---|---|---|---|---|---|---|---|---|---|
| | | ESDTSYVSLK | | GYSIFSYATK | | IANVFTNAFR | | VLAIDHLNEDQLR | |
| | | (SEQ ID NO: 31) | | (SEQ ID NO: 33) | | (SEQ ID NO: 37) | | (SEQ ID NO: 43) | |
| | | Avg. | Inter- | Avg. | Inter- | Avg. | Inter- | Avg. | Inter- |
| | | Conc. | assay | Conc. | assay | Conc. | assay | Conc. | assay |
| | | (ng/mL) | % CV | (ng/mL) | % CV | (ng/mL) | % CV | (ng/mL) | % CV |
| Patient 1 | Baseline | 218.07 | 17.3 | 167.04 | 32.8 | 55.94 | 20.0 | n/d | — |
| | 10 min | 256.19 | 21.2 | 179.55 | 36.0 | 57.48 | 26.0 | n/d | — |
| | 60 min | 295.75 | 6.9 | 240.26 | 3.2 | 56.05 | 8.5 | 1.94 | 13.5 |
| | 240 min | 252.50 | 34.3 | 209.02 | 40.6 | 14.96 | 20.5 | 6.00 | 38.4 |
| Patient 2 | Baseline | 298.74 | 45.0 | 160.64 | 33.2 | 2.53 | 39.7 | n/d | — |
| | 10 min | 341.51 | 7.4 | 199.31 | 9.8 | 7.36 | 12.3 | n/d | — |
| | 60 min | 369.92 | 17.3 | 167.54 | 31.3 | 4.78 | 33.0 | 0.66 | 19.2 |
| | 240 min | 507.80 | 8.4 | 237.99 | 20.3 | 1.32 | 15.2 | 1.52 | 17.5 |
| Patient 3 | Baseline | 5466.43 | 13.9 | 4102.82 | 11.1 | 9.99 | 17.9 | n/d | — |
| | 10 min | 4545.05 | 19.2 | 3448.23 | 16.4 | 33.44 | 18.3 | n/d | — |
| | 60 min | 4011.24 | 24.4 | 3137.97 | 21.8 | 23.86 | 21.5 | 0.49 | 23.0 |
| | 240 min | 4693.15 | 34.5 | 3908.47 | 22.3 | 7.20 | 17.6 | 1.08 | 12.5 |
| Patient 4 | Baseline | 2874.04 | 18.4 | 2856.13 | 19.9 | 2.98 | 26.7 | n/d | — |
| | 10 min | 2957.35 | 13.0 | 2304.92 | 34.6 | 14.97 | 11.4 | n/d | — |
| | 60 min | 1826.35 | 31.8 | 1589.94 | 23.5 | 10.63 | 10.0 | 0.35 | 16.0 |
| | 240 min | 2736.17 | 24.1 | 1930.59 | 21.8 | 4.23 | 16.1 | 1.65 | 21.5 |

TABLE 6
Baseline clinical characteristics of study
subjects under exercise tolerance test.

| | Ischemic patients | Non-Ischemic patients |
|---|---|---|
| | under ETT | under ETT |
| | (n = 53; cases) | (n = 58; controls) |
| Age, years | 65.1 ± 8.2 | 62.1 ± 11.2 |
| Male sex (%) | 95.3 | 89.1 |
| Caucasian Race, (%) | 89.1 | 96.9 |
| Creatine baseline | 1.2 ± 0.5 | 1.1 ± 0.2 |
| Total cholesterol baseline | 155 ± 37** | 190 ± 50 |
| Baseline Heart rate | 61.1 ± 9.7 | 65.5 ± 13.3 |
| Peak Heart rate | 124.5 ± 18.7** | 140.8 ± 27.4 |
| Previous angina history (%) | 78.9 | 31.6 |
| Previous MI history (%) | 44.7 | 15.8 |
| EKG change (%) | 79 | 18.4 |
| Image (%) | 100 | 5.7 |
| Aspirin (%) | 92 | 32 |
| Beta-blocker (%) | 87 | 45 |
| Calcium channel blocker (%) | 29 | 16 |
| Statin (%) | 89 | 58 |

TABLE 7

| | | | | Power | | |
|---|---|---|---|---|---|---|
| | | | Minimum | Min. | | |
| Significance | Coefficient of | Number | detectable | detectable | | |
| level | variation | of sample | fold | fold | 3-fold | 5-fold |
| (p-value) | (CV) of assay | pairs | change | change | change | change |
| 0.05 | 0.2 | 6 | 1.35 | 0.45 | 0.99 | 1.00 |
| | | 7 | 1.30 | 0.45 | 1.00 | 1.00 |
| | | 8 | 1.27 | 0.45 | 1.00 | 1.00 |
| | | 10 | 1.23 | 0.45 | 1.00 | 1.00 |
| | 0.3 | 6 | 1.59 | 0.41 | 0.86 | 0.95 |
| | | 7 | 1.50 | 0.42 | 0.93 | 0.98 |
| | | 8 | 1.44 | 0.42 | 0.97 | 0.99 |
| | | 10 | 1.36 | 0.43 | 0.99 | 1.00 |
| | 0.5 | 6 | 2.33 | 0.36 | 0.46 | 0.61 |
| | | 7 | 2.06 | 0.37 | 0.55 | 0.70 |
| | | 8 | 1.90 | 0.37 | 0.63 | 0.78 |
| | | 10 | 1.71 | 0.38 | 0.76 | 0.89 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting or diagnosing cardiovascular injury in a subject comprising the steps of:
a) obtaining a biological sample from the subject;
b) determining the level of expression of at least one biomarker selected from the group consisting of proteins 8-31 from Table 1B, the proteins of Table 1A, and any combinations thereof; and
c) comparing expression levels of the at least one biomarker or combination thereof in a reference or control sample;
whereby a change in the expression level of the at least one biomarker or combination thereof as compared to the reference or control is indicative of cardiovascular injury in the subject.

2. The method of claim 1, further comprising the step of additionally determining the level of expression of at least one additional biomarker selected from the group consisting of proteins 1-7 of Table 1B and any combination thereof.

3. The method of claim 1, wherein determining the level of expression of the at least one biomarker comprises detecting the expression, if any, of the polypeptide(s) encoded by said biomarker or combination thereof in the sample.

4. The method of claim 3, wherein detecting the expression of the polypeptide(s) comprises exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.

5. The method of claim 1, wherein said biological sample comprises whole blood, blood fraction, plasma, or a fraction thereof.

6. The method of claim 1, wherein the cardiovascular injury is selected from the group consisting of myocardial infarction, stable ischemic heart disease, unstable ischemic heart disease, acute coronary syndrome, ischemic cardiomyopathy, and heart failure.

7. A method for detecting or diagnosing cardiovascular injury in a subject comprising the steps of:
a) obtaining a biological sample from the subject;
b) determining the level of expression of two or more cardiovascular injury biomarkers; and
c) comparing expression levels of the two or more cardiovascular injury biomarkers in a reference or control sample;
whereby a change in the expression level of the two or more cardiovascular injury biomarkers as compared to the reference or control is indicative of cardiovascular injury in the subject.

8. The method of claim 7, wherein the two or more cardiovascular injury biomarkers are selected from the group consisting of the proteins listed in Table 1A, Table 1B, and Table 4.

9. The method of claim 7, wherein determining the level of expression of the two or more cardiovascular injury biomarkers comprises detecting the expression, if any, of the polypeptide(s) encoded by the biomarkers in the sample.

10. The method of claim 9, wherein detecting the expression of the polypeptide(s) comprises exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.

11. The method of claim 7, wherein said biological sample comprises whole blood, blood fraction, plasma, or a fraction thereof.

12. The method of claim 7, wherein the cardiovascular injury is selected from the group consisting of myocardial infarction, stable ischemic heart disease, unstable ischemic heart disease, acute coronary syndrome, ischemic cardiomyopathy, and heart failure.

13. A kit comprising in one or more containers at least one of the proteins listed in Table 1A, Table 1B, or Table 4.

14. The kit of claim 13, wherein the level of expression of the proteins is determined using the components of the kit.

15. The kit of claim 14, wherein the kit is used to generate a biomarker profile.

16. The kit of claim 15, wherein the kit optionally comprises at least one internal standard to be used to generate the biomarker profile.

17. The kit of claim 13, wherein the kit further comprises at least one pharmaceutical excipient, diluent, adjuvant, or any combination thereof.

18. A kit comprising in one or more containers at least one detectably labeled reagent that specifically recognize at least one of the proteins listed in Table 1A, Table 1B, or Table 4.

19. The kit of claim 18, wherein the at least one detectably labeled reagent is used to determine the expression level of at least one of the proteins listed in Table 1A, Table 1B, or Table 4 in a biological sample.

20. The kit of claim 19, wherein said biological sample comprises whole blood, blood fraction, plasma, or a fraction thereof.

21. A method of selecting an appropriate therapy or treatment protocol in a patient diagnosed with or suspected of having a cardiovascular injury, the method comprising
a) obtaining a biological sample from the subject;
b) determining the level of expression of at least one biomarker selected from the group consisting of proteins 8-31 from Table 1B, the proteins of Table 1A, and any combinations thereof; and
c) choosing the appropriate therapy or treatment protocol based on the level of expression of the at least one biomarker or combination thereof.

22. The method of claim 21 further comprising the step of:
d) repeating steps a) and b) on a periodic basis in order to determine whether an additional or alternative therapy or treatment protocol needs to be chosen.

23. The method of claim 22, wherein the periodic basis is selected from the group consisting of hourly, daily, weekly, or monthly.

24. A method of identifying a biomarker, the method comprising the steps of:
a) discovering one or more candidate biomarker proteins in proximal fluid or tissue;
b) qualifying the one or more discovered candidate biomarker proteins in peripheral blood of additional patient samples; and
c) verifying the qualified, discovered one or more candidate biomarker proteins.

25. The method of claim 24, wherein the discovering of the one or more candidate biomarker proteins is accomplished using liquid chromatography-tandem mass spectrometry (LC-MS/MS) with extensive fractionation.

26. The method of claim 24, wherein qualifying the one or more discovered candidate biomarker proteins is accomplished using Accurate Inclusion of Mass Screening (AIMS).

27. The method of claim 24, wherein verifying the qualified, discovered one or more candidate biomarker proteins is accomplished using targeted, qualitative a MS-based assay.

28. The method of claim 27, wherein the targeted, qualitative MS-based assay is selected from the group consisting of multiple reaction monitoring mass spectrometry (MRM-MS), SISCAPA, and combinations thereof.

29. A method for detecting or diagnosing cardiovascular injury in a subject comprising the steps of:
a) obtaining a biological sample from the subject;
b) determining the level of expression of Acyl-CoA binding protein (ACBP); and
c) comparing expression levels of the Acyl-CoA binding protein (ACBP) to a reference or control sample;
whereby a change in the expression level of Acyl-CoA binding protein (ACBP) as compared to the reference or control is indicative of cardiovascular injury in the subject.

30. The method of claim 29, further comprising the step of additionally determining the level of expression of at least one additional biomarker selected from the group consisting of proteins from Table 1A, the proteins of Table 1B, and any combination thereof.

31. The method of claim 29, wherein determining the level of expression of Acyl-CoA binding protein (ACBP) comprises detecting the expression, if any, of the polypeptide(s) encoded by Acyl-CoA binding protein (ACBP) in the sample.

32. The method of claim 31, wherein detecting the expression of the polypeptide(s) comprises exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.

33. The method of claim 29, wherein said biological sample comprises whole blood, blood fraction, plasma, or a fraction thereof.

34. The method of claim 29, wherein the cardiovascular injury is selected from the group consisting of myocardial infarction, stable ischemic heart disease, unstable ischemic heart disease, acute coronary syndrome, ischemic cardiomyopathy, and heart failure.

35. The method of claim 2, wherein determining the level of expression of the at least one biomarker comprises detecting the expression, if any, of the polypeptide(s) encoded by said biomarker or combination thereof in the sample.

36. The method of claim 35, wherein detecting the expression of the polypeptide(s) comprises exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.

37. The method of claim 30, wherein determining the level of expression of Acyl-CoA binding protein (ACBP) comprises detecting the expression, if any, of the polypeptide(s) encoded by Acyl-CoA binding protein (ACBP) in the sample.

38. The method of claim 37, wherein detecting the expression of the polypeptide(s) comprises exposing the sample to an antibody or antigen-binding fragment thereof specific to the polypeptide(s) and detecting the binding, if any, of said antibody or antigen-binding fragment to said polypeptide(s) and quantifying the level of the polypeptide(s) in the sample.
